(19) United States
(12) Patent Application Publication
(10) Pub. No.: US 2025/0282869 A1
(43) Pub. Date: Sep. 11, 2025

(54) HETEROMULTIMER BINDING DLL3 AND CD3

(71) Applicants: AMGEN INC., Thousand Oaks, CA (US); AMGEN RESEARCH (MUNICH) GMBH, Munich (DE)

(72) Inventors: Julie BAILIS, Thousand Oaks, CA (US); Tobias RAUM, Munich (DE); Matthias KLINGER, Munich (DE); Doris RAU, Munich (DE); Jennitte STEVENS, Munich (DE); Suzanne EDAVETTAL, Thousand Oaks, CA (US); Reudiger NEEF, Munich (DE); Janette WALTER, Munich (DE); Mathias KAEFER, Munich (DE); Tara ARVEDSON, Thousand Oaks, CA (US); Fernando GARCES, Thousand Oaks, CA (US)

(73) Assignees: AMGEN INC., Thousand Oaks, CA (US); AMGEN RESEARCH (MUNICH) GMBH, Munich (DE)

(21) Appl. No.: 18/861,421

(22) PCT Filed: May 4, 2023
(86) PCT No.: PCT/US2023/066606

§ 371 Date: Oct. 29, 2024

Related U.S. Application Data
(60) Provisional application No. 63/338,751, filed on May 5, 2022.

(51) Int. Cl. C07K 16/28; A61K 39/00; A61K 39/395

(52) U.S. Cl. CPC C07K 16/2809; A61K 39/39558; C07K 16/28; A61K 2039/505; C07K 2317/31; C07K 2317/52; C07K 2317/55; C07K 2317/622; C07K 2317/73; C07K 2317/92

(57) ABSTRACT

The disclosure provides a heteromultimer comprising a first heterodimer that binds to human delta-like ligand 3 (DLL3) and a second heterodimer that binds to human CD3, which can bind to DLL3-expressing cancer cells. The disclosure also provides methods of treating a DLL3-expressing cancer in a subject in need thereof, which comprises administering to the subject an effective amount of the heteromultimer or a composition comprising the heteromultimer.

### CROSS-REFERENCE TO RELATED APPLICATIONS

This application claims the benefit of U.S. Provisional Application No. 63/338,751, filed on May 5, 2022.

### FIELD

The disclosure relates to multimeric antigen-binding proteins which bind to DLL3 and CD3.

### INCORPORATION-BY-REFERENCE OF MATERIAL SUBMITTED ELECTRONICALLY

Incorporated by reference in its entirety herein is a nucleotide/amino acid sequence listing submitted concurrently herewith and identified as follows: One 90.8 kilobyte XML document named “10092-WO01-SEC.xml,” created on Apr. 25, 2023.

### BACKGROUND

Small cell lung cancer (SCLC) is an aggressive form of lung cancer with a poor prognosis and limited therapeutic options, representing about 13% of all newly diagnosed lung cancers, with more than 235,000 adults receiving a diagnosis of SCLC in the U.S. in 2021. Survival rates have remained low for several decades, with only 7% of SCLC patients surviving five years, in a large part due to the lack of new therapies to combat this form of lung cancer. Most patients present with extensive-stage disease, while about a third of patients present with limited stage disease, defined by the presence of tumors in only one side of the chest and that fit in a single radiation field. Disseminated, metastatic tumors with lymphoma-like characteristics are a hallmark of SCLC. The first known diagnosis of SCLC patients described it as a disease of the lymphatic system, and SCLC was not recognized as lung cancer until 1926, which highlights the unique nature of SCLC tumors as compared to other solid tumors.

Patients typically respond well to the current standard of care, which includes chemotherapy combined with thoracic radiation therapy (TRT), but invariably quickly relapse with chemoresistant disease, for which no therapeutic options are currently available. Recently, the addition of the anti-PD-L1 antibody atezolizumab (TECENTRIQ®) to carboplatin and etoposide chemotherapy demonstrated an improvement in overall survival (OS) in the first-line setting, leading to the approval of this regimen by the United States Food and Drug Administration (FDA) for first-line treatment of extensive-stage SCLC. Despite these therapeutic advances, prognosis in the relapsed refractory (RR) setting is extremely poor, with rapid disease progression and short median survival of less than six months. Furthermore, SCLC patients have high rates of comorbidities, including hypertension, cardiac disease, diabetes and paraneoplastic syndromes. These, coupled with the typically advanced age of SCLC patients, impact the ability of patients to endure harsh chemotherapy regimens, further limiting treatment options.

Delta-like ligand 3 (DLL3) is an inhibitory Notch ligand that is highly expressed in SCLC and other neuroendocrine tumors but minimally expressed in normal tissues. In one study, approximately 86% of SCLC tumors analyzed showed evidence of DLL3 expression by RNA-seq (Giffin et al., Clin. Cancer Res., 27 (5): 1526-1537 (2021). doi: 10.1158/1078-0432.CCR-20-2845). In contrast, only a few normal cell types have been shown to express DLL3 (e.g., neurons, pancreatic islet cells, and pituitary cells), and such expression was predominantly cytoplasmic. Recent studies have reported that DLL3 is also expressed in other tumor types of neuroendocrine origin, including melanoma, glioblastoma multiforme, neuroendocrine prostate cancer (NEPC), and large cell neuroendocrine lung tumors (Giffin et al., Clin. Cancer Res., 27 (5): 1526-1537 (2021) and Saunders et al., Sci Transl Med., 7 (302): 302ra136. doi: 10.1126/scitranslmed.aac9459 (2015).

There remains a need for compositions and methods that more efficiently target and treat neuroendocrine cancers such as, but not limited to, small cell lung cancer.

### BRIEF SUMMARY

The disclosure provides a heteromultimer comprising a first heterodimer that binds to human delta-like ligand 3 (DLL3) and a second heterodimer that binds to human cluster of differentiation (CD) 3, wherein (a) the first heterodimer comprises: a heavy chain (HC) comprising the amino acid sequence of SEQ ID NO: 54 or SEQ ID NO: 58; and a light chain (LC) comprising the amino acid sequence of SEQ ID NO: 55 or SEQ ID NO: 59; and (b) the second heterodimer comprises: a heavy chain comprising the amino acid sequence of SEQ ID NO: 56 or SEQ ID NO: 132; and a light chain comprising the amino acid sequence of SEQ ID NO: 57.

In some embodiments, (a) the first heterodimer comprises a heavy chain comprising the amino acid sequence of SEQ ID NO: 54 and a light chain comprising the amino acid sequence of SEQ ID NO: 55; and (b) the second heterodimer comprises a heavy chain comprising the amino acid sequence of SEQ ID NO: 56 and a light chain comprising the amino acid sequence of SEQ ID NO: 57.

In some embodiments, (a) the first heterodimer comprises a heavy chain comprising the amino acid sequence of SEQ ID NO: 54 and a light chain comprising the amino acid sequence of SEQ ID NO: 55; and (b) the second heterodimer comprises a heavy chain comprising the amino acid sequence of SEQ ID NO: 132 and a light chain comprising the amino acid sequence of SEQ ID NO: 57.

In some embodiments, (a) the first heterodimer comprises a heavy chain comprising the amino acid sequence of SEQ ID NO: 58 and a light chain comprising the amino acid sequence of SEQ ID NO: 59; and (b) the second heterodimer comprises a heavy chain comprising the amino acid sequence of SEQ ID NO: 56 and a light chain comprising the amino acid sequence of SEQ ID NO: 57.

In some embodiments, (a) the first heterodimer comprises a heavy chain comprising the amino acid sequence of SEQ ID NO: 58 and a light chain comprising the amino acid sequence of SEQ ID NO: 59; and (b) the second heterodimer comprises a heavy chain comprising the amino acid sequence of SEQ ID NO: 132 and a light chain comprising the amino acid sequence of SEQ ID NO: 57.

The disclosure also provides a composition comprising the aforementioned heteromultimer and a pharmaceutically acceptable carrier. In some embodiments, the composition comprises the aforementioned heteromultimer for use in a method of treating a DLL3-expressing cancer, such as a neuroendocrine cancer (e.g., small cell lung cancer (SCLC), neuroendocrine prostate cancer (NEPC), or neuroblastoma).

Also provided is a kit comprising the above-mentioned compositions and instructions for use.

The disclosure provides a method of inhibiting growth of DLL3-expressing cancer cells, which comprises contacting a population of DLL3-expressing cancer cells and CD3-expressing T cells with an effective amount of the aforementioned heteromultimer or composition.

The disclosure further provides a method of treating a DLL3-expressing cancer in a subject in need thereof, which comprises administering to the subject an effective amount of the aforementioned heteromultimer or composition.

The disclosure also provides a nucleic acid sequence encoding the aforementioned heteromultimer.

Also provided is use of the aforementioned heteromultimer in the manufacture of a medicament for the treatment of a DLL3-expressing cancer.

### BRIEF DESCRIPTION OF THE DRAWINGS

FIGS. 1A-1D are schematic diagrams of DLL3-binding heteromultimers in the following formats: B1mAb (2+1) (FIG. 1A), AmAb (1+1) (FIG. 1B), N1mAb (2+1) (FIG. 1C), and Hetero IgG (1+1) (FIG. 1D).

FIGS. 2A and 2B are graphs showing pharmacokinetics (PK) of a DLL3-binding heteromultimer (“DLL3_2”) in various formats. The hetero IgG version (“het IgG”) showed a mAb-like PK profile in FcRn-transgenic mice following intravenous (IV) administration at 1 mg/kg (FIG. 2A; square with dashed line, B1 mAb; circle with dashed line, N1 mAb; star with dashed line, het IgG) and subcutaneous (SC) administration at 1 mg/kg (FIG. 2B; star with dashed line, het IgG; diamond with dashed line, B1 mAb; triangle with solid line, N1 mAb).

FIG. 3 is a graph showing pharmacokinetics (PK) of a DLL3-binding heteromultimer (“DLL3_1”) in various formats (circle with solid line, AmAb; diamond with solid line, B1 mAb; diamond with dashed line, N1 mAb; square with solid line, N1 mAb; cross with dashed line, het IgG; star with dashed line, B1 mAb) in FcRn-transgenic mice. The hetero IgG version (“het IgG”) exhibited a more favorable PK profile in FcRn-transgenic mice following intravenous (IV) administration.

FIGS. 4A and 4B are graphs showing mean serum concentrations in female cynomolgus monkeys following administration of the DLL3-binding heteroIgG molecule DLL3_2 either subcutaneously (FIG. 4A) or intravenously (FIG. 4B) using three different assays as described in Example 4.

### DETAILED DESCRIPTION

The present disclosure is predicated, at least in part, on the development of a multimeric protein that targets and kills DLL3-expressing tumor cells (e.g., SCLC cells, neuroendocrine prostate cancer cells, or other neuroendocrine cancer cells). The multimeric protein further comprises a binding domain that engages and activates T cells, leading to tumor-specific cytotoxicity. Thus, the multimeric protein described herein may also be referred to as a “multi-chain T cell engager (mcTCE).”

In some embodiments, the disclosure provides a heteromultimer comprising a first heterodimer that binds to human DLL3 and a second heterodimer that binds to human CD3. For example, the first heterodimer may comprise a heavy chain (HC) comprising the amino acid sequence of SEQ ID NO: 54 or SEQ ID NO: 58; and a light chain (LC) comprising the amino acid sequence of SEQ ID NO: 55 or SEQ ID NO: 59; and the second heterodimer may comprise a heavy chain comprising the amino acid sequence of SEQ ID NO: 56 or SEQ ID NO: 132; and a light chain comprising the amino acid sequence of SEQ ID NO: 57. Without being limited by theory, it is contemplated that the heteromultimers described herein exhibit advantageous manufacturing properties. Such advantageous properties include, for example, high expression levels, production yields, and stability, as well as reduced undesirable mispaired species.

### Definitions

To facilitate an understanding of the present technology, several terms and phrases are defined below. Additional definitions are set forth throughout the detailed description.

A “multimeric protein,” as used herein, refers to a protein containing more than one separate polypeptide or protein chains associated with each other to form a single protein in vitro or in vivo. A multimeric protein may comprise more than one polypeptide of the same kind to form a “homomultimer.” Alternatively, a multimeric protein may also be composed of more than one polypeptide of distinct sequences to form a “heteromultimer.” Thus, a “heteromultimer” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where more than two polypeptides are present.

The term “antigen-binding protein,” as used herein, refers to a proteinaceous molecule that specifically binds to an antigen. For example, an antigen-binding protein may comprise an antibody or an antigen-binding fragment thereof. An antigen-binding protein typically comprises the heavy chain variable region (VH) and/or the light chain variable region (VL) of an antibody, or comprises domains derived therefrom. In some embodiments, an antigen-binding protein comprises the minimum structural requirements of an antibody which allow for immunospecific target binding. This minimum requirement may be defined by, for example, the presence of at least three light chain complementarity determining regions (CDRs) (i.e., CDR1, CDR2 and CDR3 of the VL region) and/or three heavy chain CDRs (i.e., CDR1, CDR2 and CDR3 of the VH region), and ideally of all six CDRs. It is within the knowledge of a skilled person where (and in which order) those CDRs are located in the antigen-binding protein.

As used herein, the term “antibody” refers to an immunoglobulin of any isotype with specific binding to the target antigen; an antibody may be a polyclonal or monoclonal antibody, a chimeric antibody, a humanized antibody, a human antibody, etc. In a native antibody, a heavy chain comprises a variable region, VH, and three constant regions, CH1, CH2, and CH3. The VH domain is at the amino-terminus of the heavy chain, and the CH3 domain is at the carboxy-terminus. In a native antibody, a light chain comprises a variable region, VL, and a constant region, CL. The variable region of the light chain is at the amino-terminus of the light chain. In a native antibody, the variable regions of each light/heavy chain pair typically form the antigen-binding site. The constant regions are typically responsible for effector function. A native antibody is a tetramer of two full-length heavy chains and two full-length light chains.

In a human antibody, CH1 means a region having the amino acid sequence at positions 118 to 215 of the EU index or EU numbering system, which is based on the sequential numbering of the first human IgG1 sequenced (i.e., the “EU antibody”) (Edelman et al., Proc Natl Acad Sci USA, 63 (1): 78-85 (1969)). A highly flexible amino acid region called a “hinge region” exists between CH1 and CH2. CH2 represents a region having the amino acid sequence at positions 231 to 340 of the EU index, and CH3 represents a region having the amino acid sequence at positions 341 to 446 of the EU index.

“CL” represents a constant region of a light chain. In the case of a κ chain of a human antibody, CL represents a region having the amino acid sequence at positions 108 to 214 of the EU index. In a λ chain, CL represents a region having the amino acid sequence at positions 108 to 215.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen binding site. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); or Chothia, C., and Lesk, A. M. (1987) J. Mol. Biol., 196:901-917. In some embodiments, the CDRs of an antigen binding protein are defined according to the definition of Kabat or Chothia. In the present application, the term “CDR” refers to a CDR from either the light or heavy chain, unless otherwise specified.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. The term “monoclonal antibody,” as used herein, refers to an antibody produced by a single clone of B lymphocytes that is directed against a single epitope on an antigen. Monoclonal antibodies typically are produced using hybridoma technology, as first described in Kohler and Milstein, Eur. J. Immunol., 5:511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352:624-628 (1991)); and Marks et al., J. Mol. Biol., 222:581-597 (1991)), or produced from transgenic mice carrying a fully human immunoglobulin system (see, e.g., XENOMOUSE™ mouse, Green et al. (1994) Nature Genetics 7:13-21, US 2003-0070185, WO 96/34096, and WO 96/33735). In contrast, “polyclonal” antibodies are antibodies that are secreted by different B cell lineages within an animal. Polyclonal antibodies are a collection of immunoglobulin molecules that recognize multiple epitopes on the same antigen.

The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. In exemplary aspects, the antigen binding protein of the present disclosure comprises an antigen binding antibody fragment. As used herein, the term “antigen binding antibody fragment” refers to a portion of an antibody molecule that is capable of binding to the antigen of the antibody and is also known as “antigen-binding fragment” or “antigen-binding portion.” In exemplary instances, the antigen binding antibody fragment is a Fab fragment or a F(ab′)2 fragment.

The architecture of antibodies has been exploited to create a growing range of alternative formats that span a molecular-weight range of at least about 12-150 kDa and has a valency (n) range from monomeric (n=1), to dimeric (n=2), to trimeric (n=3), to tetrameric (n=4), and potentially higher; such alternative formats are referred to herein as “antibody protein products.” Antibody protein products include those based on the full antibody structure and those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. A peptibody or peptide-Fc fusion is yet another antibody protein product. The structure of a peptibody consists of a biologically active peptide grafted onto an Fc domain. Peptibodies are well-described in the art (see, e.g., Shimamoto et al., mAbs 4 (5): 586-591 (2012)).

The antigen binding heteromultimer of the present disclosure may comprise any one of the above-described antibody protein products. In exemplary aspects, the antigen binding heteromultimer of the present disclosure comprises any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, or BsAb conjugate.

In certain aspects, the multimeric antigen binding proteins of the present disclosure may be “bispecific,” meaning that they are capable of specifically binding to two different antigens. In another aspect, the multimeric antigen binding proteins of the present disclosure may be “trispecific,” meaning that they are capable of specifically binding to three different antigens. In another aspect, the multimeric antigen binding proteins of the present disclosure may be “tetraspecific,” meaning that they are capable of specifically binding to four different antigens.

As used herein, an antigen binding protein “specifically binds” to a target antigen when it has a significantly higher binding affinity for, and consequently is capable of distinguishing, that antigen, compared to its affinity for other unrelated proteins, under similar binding assay conditions. Antigen binding proteins that specifically bind an antigen may have an equilibrium dissociation constant (K_{D})≤1×10⁻⁶ M. In exemplary aspects, the K_{D} of the antigen binding proteins provided herein is micromolar, nanomolar, picomolar or femtomolar. The antigen binding protein specifically binds antigen with “high affinity” when the K_{D} is ≤3×108 M. In some embodiments, the antigen binding proteins of the disclosure bind to target antigen(s) with a K_{D} of ≤100 nm (e.g., 90 nm, 80 nm, 70 nm, 60 nm, 50 nm, 40 nm, 30 nm, 20 nm, 10 nm, 5 nm, or a range defined by any two of the foregoing values). In other embodiments, the antigen binding proteins of the disclosure bind to target antigen(s) with a K_{D} of about 10 nm-30 nm (e.g., about 15 nm, 20 nm, or 25 nm).

Affinity may be determined using a variety of techniques, an example of which is enzyme-linked immunosorbent assay (ELISA). In various embodiments, affinity is determined by a surface plasmon resonance assay (e.g., BIACORE®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity may be determined by a kinetic method, such as a Kinetic Exclusion Assay (KinExA) as described in Rathanaswami et al., Analytical Biochemistry, 373:52-60 (2008). Using a KinExA assay, the equilibrium dissociation constant (K_{D} in M) and the association rate constant (ka in M⁻¹s⁻¹) can be measured. The dissociation rate constant (k_{d} in s⁻¹) can be calculated from these values (K_{D}×kₐ). In other embodiments, affinity is determined by an equilibrium/solution method. In certain embodiments, affinity is determined by an on-cell binding assay using flow cytometry. In certain embodiments of the disclosure, the antigen binding protein specifically binds to target antigen(s) expressed by a mammalian cell (e.g., CHO, HEK 293, Jurkat), with a K_{D} of 20 nM (2.0×10⁻⁸ M) or less, K_{D} of 10 nM (1.0×10⁻⁸ M) or less, K_{D} of 1 nM (1.0×10⁻⁹ M) or less, K_{D} of 500 pM (5.0×10⁻¹⁰ M) or less, K_{D} of 200 pM (2.0×10⁻¹⁰ M) or less, K_{D} of 150 pM (1.50×10⁻¹⁰ M) or less, K_{D} of 125 pM (1.25×10⁻¹⁰ M) or less, K_{D} of 105 pM (1.05×10⁻¹⁰ M) or less, K_{D} of 50 pM (5.0×10⁻¹¹ M) or less, or K_{D} of 20 pM (2.0×10⁻¹¹ M) or less, as determined by a Kinetic Exclusion Assay (Rathanaswami et al., supra). In some embodiments, the multimeric antigen binding proteins described herein exhibit desirable characteristics such as binding avidity as measured by k_{d} for target antigen(s) of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} for target antigen(s) of about 10⁻⁹, 10⁻¹⁰, 10⁻¹¹, 10⁻¹², 10⁻¹³, 10⁻¹⁴, 10⁻¹⁵, 10⁻¹⁶ M or lower (lower values indicating higher binding affinity).

In certain embodiments of the disclosure, the antigen binding proteins may be multivalent. The valency of the binding protein denotes the number of individual antigen binding domains within the binding protein. In some embodiments, a bispecific antigen binding protein may be multivalent. For instance, in certain embodiments, a bispecific antigen binding protein may be tetravalent by comprising four antigen-binding domains: two antigen-binding domains binding to a first target antigen and two antigen-binding domains binding to a second target antigen. A tetraspecific antigen binding protein is tetravalent and comprises four antigen-binding domains: one to antigen-binding domain binding to a first target antigen, one antigen-binding domain binding to a second target antigen, one to antigen-binding domain binding to a third target antigen, and one antigen-binding domain binding to a fourth target antigen.

As used herein, the term “antigen binding domain,” which is used interchangeably with “binding domain,” refers to the region of the antigen binding protein that contains the amino acid residues that interact with the antigen and confer on the antigen binding protein its specificity and affinity for the antigen. In some embodiments, the binding domain may be derived from the natural ligands of the target antigen(s). As used herein, the term “target antigen(s)” refers to a first target antigen and/or a second target antigen of a bispecific molecule and also refers to a first target antigen, a second target antigen, a third target antigen, and/or a fourth target antigen of a tetraspecific molecule.

The term “immunoglobulin domain,” as used herein, refers to a peptide comprising an amino acid sequence similar to that of immunoglobulin and comprising approximately 100 amino acid residues including at least two cysteine residues. Examples of immunoglobulin domains include VH, CH1, CH2, and CH3 of an immunoglobulin heavy chain, and VL and CL of an immunoglobulin light chain. In addition, the immunoglobulin domain is found in proteins other than immunoglobulin. Examples of the immunoglobulin domain in proteins other than immunoglobulin include an immunoglobulin domain included in a protein belonging to an immunoglobulin super family, such as a major histocompatibility complex (MHC), CD1, B7, T-cell receptor (TCR), and the like. Any of the immunoglobulin domains can be used as an immunoglobulin domain for the multimeric proteins described herein.

The binding domains that specifically bind to target antigen(s) can be derived a) from known antibodies to these antigens or b) from new antibodies or antibody fragments obtained by de novo immunization methods using the antigen proteins or fragments thereof, by phage display, or other routine methods. The antibodies from which the binding domains for the antigen binding proteins are derived can be monoclonal antibodies, polyclonal antibodies, recombinant antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

As used herein, the terms “stability” and “stabilizing” are defined as the maintenance of the chemical or physical integrity and/or bioactivity of the antigen-binding polypeptide or protein over a period of time. Stabilizing an antigen-binding polypeptide or protein includes the prevention or delay of degradation or deterioration of the antigen-binding polypeptide or protein from its biologically and/or therapeutically active form to an inactive form. Instability may arise from events such as aggregation, denaturation, fragmentation, or chemical modifications such as oxidation, cross-linking, deamidation and reactions with other components featured in the composition comprising the antigen-binding polypeptide or protein.

The stability of an antigen-binding protein or polypeptide may be characterized using known methods in the art, including but not limited to, measurement of biological activity such as antigen-binding activity with immunoassay techniques such as ELISA, or other techniques of determining purity or physical/chemical changes to the antigen-binding protein or polypeptide such as size exclusion chromatography, capillary gel electrophoresis, circular dichroism, or mass spectrometry. Stability is determined by comparison of measurements obtained via these types of characterization methods at an initial time point, such as at the time of formulation or preparation of the composition (i.e., as the case may be, the suspension or dispersion), and those obtained at a later time point, that is, after storage in a given environment or condition.

The term “CD3 receptor complex,” us used herein, refers to a protein complex composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The most preferred epitope of CD3 epsilon is comprised within amino acid residues 1-27 of the human CD3 epsilon extracellular domain.

### Agents Targeting DLL3

Delta-like ligand 3 (DLL3) is a non-canonical Notch ligand expressed primarily during embryonic development that functions during somitogenesis. DLL3 accumulates in the Golgi in normal tissues (Geffers et al, J Cell Biol. 178:465-476 (2007)). DLL3 was identified as a tumor-associated antigen and a target for T cell-based therapies by analyzing its differential expression in SCLC tumors and a large panel of normal tissues (Saunders et al., supra; Giffin et al., J Thorac Oncol., 13 (10): S971 (2018)). The human DLL3 protein comprises several extracellular domains: signal peptide, N-terminus, DSL, EGF1, EGF2, EGF3, EGF4, EGF5, EGF6, and a membrane proximal domain. Exemplary amino acid sequences of human DLL3 include, e.g., UniProt Q9NYJ7 and NCBI Reference Sequence: NP_058637.1.

One example of an agent targeting DLL3 is a bispecific T cell engaging antigen-binding polypeptide that binds DLL3 and CD3, such as a BiTE® molecule. BiTE® molecules are recombinant proteins comprised of two flexibly linked binding domains, each domain derived from antibodies. One binding domain of a BiTE® molecule is specific for a tumor-associated surface antigen (such as DLL3); the second binding domain is specific for CD3, a subunit of the T cell receptor complex on T cells. By their design, BiTE® molecules are uniquely suited to transiently connect T cells with target cells and, at the same time, potently activate the inherent cytolytic potential of T cells against target cells. See e.g., WO 99/54440, WO 2005/040220, and WO 2008/119567.

AMG 757 (tarlatamab) is a half-life-extended (HLE) BiTE® molecule developed for the treatment of SCLC. The activity of AMG 757 requires the simultaneous binding to both target cells (DLL3+ cells) and T cells. The pharmacological effect of AMG 757 is mediated by specific redirection of previously primed cytotoxic CD8+ or CD4+T lymphocytes to kill DLL3+ cells. AMG 757 showed antitumor activity in SCLC patients in a Phase 1 study, and clinical evaluation is ongoing (see, e.g., ClinicalTrials.gov Identifier: NCT03319940 and Pax-Ares et al., J Clin Oncol, JCO2202823. doi: 10.1200/JCO.22.02823 (2023)).

In some embodiments, the heteromultimers provided herein are heterodimeric antibodies (used interchangeably herein with “hetero immunoglobulins” or “hetero Igs”), which are antibodies comprising two different light chains and two different heavy chains. An exemplary heteromultimer encompassed by the disclosure comprises a first heterodimer that binds to human DLL3 and a second heterodimer that binds to human CD3. In some embodiments, the first heterodimer comprises a heavy chain (HC) comprising the amino acid sequence of SEQ ID NO: 54 or SEQ ID NO: 58 and a light chain (LC) comprising the amino acid sequence of SEQ ID NO: 55 or SEQ ID NO: 59; and the second heterodimer comprises a heavy chain comprising the amino acid sequence of SEQ ID NO: 56 or SEQ ID NO: 132; and a light chain comprising the amino acid sequence of SEQ ID NO: 57. For example, the first heterodimer may comprise a heavy chain comprising the amino acid sequence of SEQ ID NO: 54 and a light chain comprising the amino acid sequence of SEQ ID NO: 55. Alternatively, the first heterodimer may comprise a heavy chain comprising the amino acid sequence of SEQ ID NO: 58 and a light chain comprising the amino acid sequence of SEQ ID NO: 59. In some embodiments, for example, the second heterodimer may comprise a heavy chain comprising the amino acid sequence of SEQ ID NO: 56 and a light chain comprising the amino acid sequence of SEQ ID NO: 57. In other embodiments, for example, the second heterodimer may comprise a heavy chain comprising the amino acid sequence of SEQ ID NO: 132 and a light chain comprising the amino acid sequence of SEQ ID NO: 57. In some embodiments, the first heterodimer may comprise a heavy chain comprising the amino acid sequence of SEQ ID NO: 60. In further embodiments, the second heterodimer may comprise a heavy chain comprising the amino acid sequence of SEQ ID NO: 133.

Thus, an exemplary heteromultimer provided herein may comprise a first heterodimer comprising a heavy chain amino acid sequence of SEQ ID NO: 54 and a light chain amino acid sequence of SEQ ID NO: 55; and a second heterodimer comprising a heavy chain amino acid sequence of SEQ ID NO: 56 and a light chain amino acid sequence of SEQ ID NO: 57. In other embodiments, an exemplary heteromultimer provided herein may comprise a first heterodimer comprising a heavy chain amino acid sequence of SEQ ID NO: 54 and a light chain amino acid sequence of SEQ ID NO: 55; and a second heterodimer comprising a heavy chain amino acid sequence of SEQ ID NO: 132 and a light chain amino acid sequence of SEQ ID NO: 57. In some embodiments, an exemplary heteromultimer provided herein comprises a first heterodimer comprising a heavy chain amino acid sequence of SEQ ID NO: 58 and a light chain amino acid sequence of SEQ ID NO: 59; and a second heterodimer comprising a heavy chain amino acid sequence of SEQ ID NO: 56 and a light chain amino acid sequence of SEQ ID NO: 57. In other embodiments, an exemplary heteromultimer provided herein comprises a first heterodimer comprising a heavy chain amino acid sequence of SEQ ID NO: 58 and a light chain amino acid sequence of SEQ ID NO: 59; and a second heterodimer comprising a heavy chain amino acid sequence of SEQ ID NO: 132 and a light chain amino acid sequence of SEQ ID NO: 57.

In some embodiments, the heteromultimer binds to the EGFR5 and EGFR6 extracellular domains of the human DLL3 protein.

In some embodiments, the disclosed heteromultimers may comprise a first heterodimer comprising a heavy chain amino acid sequence which is at least 90% identical (e.g., at least 91%, at least 92%, at least 93%, at least 94%, at least 95%, at least 96%, at least 97%, at least 98%, at least 99%, or 100% identical) to SEQ ID NO: 54 or SEQ ID NO: 58, and/or a light chain amino acid sequence which is at least 90% identical (e.g., at least 91%, at least 92%, at least 93%, at least 94%, at least 95%, at least 96%, at least 97%, at least 98%, at least 99%, or 100% identical) to SEQ ID NO: 55 or SEQ ID NO: 59. In other embodiments, the disclosed heteromultimers may comprise a second heterodimer comprising a heavy chain amino acid sequence which is at least 90% identical (e.g., at least 91%, at least 92%, at least 93%, at least 94%, at least 95%, at least 96%, at least 97%, at least 98%, at least 99%, or 100% identical) to SEQ ID NO: 56 or SEQ ID NO: 132, and/or a light chain amino acid sequence which is 90% identical (e.g., at least 91%, at least 92%, at least 93%, at least 94%, at least 95%, at least 96%, at least 97%, at least 98%, at least 99%, or 100% identical) to SEQ ID NO: 57.

Nucleic acid or amino acid sequence “identity” can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL Omega, T-Coffee, and ALIGN (for alignment of nucleic acid and amino acid sequences), BLAST programs (e.g., BLAST 2.13, BL2SEQ, and later versions thereof) and FASTA programs (e.g., FASTA3×, FASTM, and S SEARCH) (for sequence alignment and sequence similarity searches). Sequence alignment algorithms also are disclosed in, for example, Altschul et al., J. Molecular Biol, 275 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 27 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

The heteromultimers described herein can comprise any immunoglobulin constant region. The term “constant region” as used herein refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes. In some embodiments, the heteromultimers disclosed herein are of the IgG1 or IgG4 isotype.

The natural formation of Fc regions of IgG molecules involves the assembly of two matching Fc chains, independent of the sequences in the antigen-binding (Fab) arms. As discussed above, bispecific antibodies have target specificity to two or more different antigens. This is due to the two Fab arms having different sequences. As a result, bispecific molecules with a natural Fc region are prone to assemble heavy chain molecules of three main species: a monoclonal antibody that binds to one antigen, a monoclonal antibody that binds to a second antigen, and a bispecific antibody that binds to both antigens.

In some embodiments, two different heavy chains are used to form the first and second heterodimers of the present disclosure. To facilitate assembly of the light and heavy chains into a heterodimeric antibody, the light chains and/or heavy chains from each antibody may be engineered to reduce the formation of mispaired molecules. For example, one approach to promote heterodimer formation over homodimer formation is the so-called “knobs-into-holes” method, which involves introducing mutations into the CH3 domains of two different antibody heavy chains at the contact interface. Specifically, one or more bulky amino acids in one heavy chain are replaced with amino acids having short side chains (e.g., alanine or threonine) to create a “hole,” whereas one or more amino acids with large side chains (e.g. tyrosine or tryptophan) are introduced into the other heavy chain to create a “knob.” When the modified heavy chains are co-expressed, a greater percentage of heterodimers (knob-hole) are formed as compared to homodimers (hole-hole or knob-knob). The “knobs-into-holes” methodology is described in detail in WO 96/027011; Ridgway et al., Protein Eng., Vol. 9:617-621, 1996; and Merchant et al., Nat, Biotechnol., Vol. 16:677-681, 1998.

Another approach for promoting heterodimer formation to the exclusion of homodimer formation entails utilizing an electrostatic steering mechanism (see Gunasekaran et al., J. Biol. Chem., Vol. 285:19637-19646, 2010). This approach involves introducing or exploiting charged residues in the CH3 domain in each heavy chain so that the two different heavy chains associate through opposite charges that cause electrostatic attraction. Homodimerization of the identical heavy chains are disfavored because the identical heavy chains have the same charge and therefore are repelled. This same electrostatic steering technique can be used to prevent mispairing of light chains with the non-cognate heavy chains by introducing residues having opposite charges in the correct light chain-heavy chain pair at the binding interface. The electrostatic steering technique and suitable charge pair mutations (CPM) for promoting heterodimers and correct light chain/heavy chain pairing is described in, e.g., WO 2009/089004, WO 2014/081955, and WO 2021/092355.

In embodiments in which the heteromultimeric antigen binding proteins of the disclosure comprise a first light chain (LC1) and first heavy chain (HC1) from a first antibody that specifically binds to a first target antigen and a second light chain (LC2) and second heavy chain (HC2) from a second antibody that specifically binds to target 2, HC1 or HC2 may comprise one or more amino acid substitutions to replace a positively-charged amino acid with a negatively-charged amino acid. For instance, in one embodiment, the CH3 domain of HC1 or the CH3 domain of HC2 comprises an amino acid sequence differing from a wild-type IgG amino acid sequence such that one or more positively-charged amino acids (e.g., lysine, histidine and arginine) in the wild-type human IgG amino acid sequence are replaced with one or more negatively-charged amino acids (e.g., aspartic acid and glutamic acid) at the corresponding position(s) in the CH3 domain.

In exemplary embodiments, the heavy chain constant region of the first heterodimer disclosed herein may comprise amino acid substitutions at positions 183, 392, 409, and/or 439, and the second heterodimer may comprise amino acid substitutions at positions 183, 356, and/or 399, as numbered according to the EU index. In some embodiments, heavy chain constant region of the first heterodimer comprises the amino acid substitutions S183K, K392D, K409D, and K439D, and the heavy chain constant region of the second heterodimer comprises the amino acid substitutions S183E, E356K, and D399K. In other embodiments, the light chain constant region of the first heterodimer and the second heterodimer comprises an amino acid substitution at position 176, such as, e.g., S176K. It will be appreciated that other amino acid modifications may be made to the heteromultimers described herein to extend half-life, improve stability and/or developability. Such modifications include, but are not limited to, a YTE and/or SEFL2 modification (Dall'Acqua et al., The Journal of Immunology, 169:5171-5180 (2002); Jacobsen et al., The Journal of Biological Chemistry, 292 (3): 1865-1875 (2017)). As discussed above, the heteromultimers described herein exhibit advantageous manufacturing properties, including, for example, high expression levels and production yields, increased stability, and reduced undesirable mispaired species.

### Compositions and Kits

The disclosure provides a composition comprising the heteromultimer described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the heteromultimer. Any suitable carrier can be used within the context of the disclosure, and such carriers are well known in the art. The choice of carrier will be determined, in part, by the particular use of the composition (e.g., administration to a human) and the particular method used to administer the composition.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000); and Remington's Pharmaceutical Sciences, Sixteenth Edition, E. W. Martin, Mack Publishing Co., Easton, Pa., (1980).

In exemplary aspects, the composition may comprise components that are nontoxic to recipients at the dosages and concentrations employed. For example, a composition may comprise an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents; tonicity enhancing agents (such as alkali metal halides, preferably sodium or potassium chloride, mannitol sorbitol); delivery vehicles; diluents; excipients and/or pharmaceutical adjuvants (see, e.g., Remington's Pharmaceutical Sciences, Sixteenth Edition, E. W. Martin, Mack Publishing Co., Easton, Pa., (1980))

The compositions can be formulated to achieve a physiologically compatible pH. In some embodiments, for example, the pH of the composition may be between about 4 to about 8 (e.g., about 4.5, 5.0, 5.5, 6.0, 6.5, 7.0, 7.5, or a range defined by any two of the foregoing values).

In some embodiments, the compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal, and intramuscular injection or infusion. In some embodiments, the composition is formulated for intravenous delivery. In such an embodiment, the composition may include a lipid-based delivery vehicle. In other embodiments, the composition is formulated for subcutaneous or transdermal delivery.

In some embodiments, the composition comprises an effective amount of a heteromultimer described herein, such as a “therapeutically effective amount.” A “therapeutically effective amount” is an amount sufficient to produce a beneficial or desired clinical result. In some embodiments, a therapeutically effective amount is an amount sufficient to kill DLL3-expressing tumor cells in a subject. In other embodiments, a therapeutically effective amount of the heteromultimer desirably produces a decrease in severity of disease (e.g., cancer) symptoms, an increase in frequency or duration of disease symptom-free periods or a prevention of impairment or disability due to the disease affliction. In some embodiments, a therapeutically effective amount of the heteromultimer inhibits cancer cell growth or tumor growth by at least about 20%, at least about 40%, at least about 50%, at least about 60%, at least about 70%, at least about 80%, or at least about 90% relative to untreated subjects. The ability of a compound to inhibit tumor growth may be evaluated in an animal model predictive of efficacy in human tumors.

A typical dose may range from about 1 ug up to about 500 mg or more, depending on the factors mentioned above. For example, a daily parenteral dose can be about 2 mg or greater, such as about 3 mg to about 500 mg, about 10 mg to about 200 mg, or about 50 mg to about 100 mg. It will be appreciated that doses below or above these exemplary ranges are within the scope of the disclosure, however. In some embodiments, the dose of heteromultimer present in the composition may be about 100 mg or less (e.g., about 90 mg, about 80 mg, about 70 mg, about 60 mg, about 50 mg, about 40 mg, about 30 mg, about 20 mg, about 10 mg, or a range defined by any two of the foregoing values). In other embodiments, the dose of heteromultimer present in the composition may be about 10 mg or less (e.g., about 9 mg, about 5 mg, about 1 mg, about 0.5 mg, about 100 ug, about 1 ug, or a range defined by any two of the foregoing values) In some embodiments, a composition comprising the heteromultimer, or the heteromultimer itself, may be administered to a subject (e.g., a human) at a dose of about 3 mg to about 100 mg (e.g., about 3 mg, about 5 mg, about 10 mg, 15 mg, about 25 mg, about 35 mg, about 45 mg, about 55 mg, about 65 mg, about 75 mg, about 85 mg, about 95 mg, or a range defined by any two of the foregoing values) at least once a week. For example, a composition comprising the heteromultimer, or the heteromultimer itself, may be administered to a human at least once every two weeks, at least once every three weeks, at least once every four weeks, etc.

The heteromultimer may be provided in the form of a kit, i.e., a packaged combination of reagents in predetermined amounts with instructions for use. In exemplary aspects, the kit may comprise the heteromultimer, or a composition comprising same, in a container. In exemplary aspects, the heteromultimer, or a composition comprising same, is provided in the kit as a unit dose. The term “unit dose,” as used herein, refers to a discrete amount dispersed in a suitable carrier. In exemplary aspects, the unit dose is an amount sufficient to provide a subject with a desired effect, e.g., a therapeutically effective amount as described above. In exemplary aspects, the kit may comprise several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the heteromultimer, or a composition comprising same, is pre-packaged in a ready to use form, e.g., a syringe, an intravenous bag, etc. In exemplary aspects, the ready to use form is for a single use. In exemplary aspects, the kit comprises multiple single use, ready to use forms of the disclosed heteromultimer, or composition comprising same. In some aspects, the kit may further comprise other therapeutic or diagnostic agents or pharmaceutically acceptable carriers (e.g., solvents, buffers, diluents, etc.), including any of those described herein.

### Nucleic Acids and Vectors

The disclosure also provides one or more nucleic acid sequences that encode the heteromultimer described herein. The term “nucleic acid sequence” encompasses a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

In some aspects, the disclosure provides one or more nucleic acid sequences encoding the heavy and light chains of the first heterodimer. For example, a first nucleic acid sequence may encode the heavy chain of the first heterodimer, and a second nucleic acid sequence distinct from the first nucleic acid sequence may encode the light chain of the first heterodimer. Alternatively, both the heavy and light chains of the first heterodimer may be encoded by a single nucleic acid sequence. Similarly, a first nucleic acid sequence may encode the heavy chain of the second heterodimer, and a second distinct nucleic acid sequence may encode the light chain of the second heterodimer. Alternatively, both the heavy and light chains of the second heterodimer may be encoded by a single nucleic acid sequence. In other aspects, a single nucleic acid sequence may encode the heavy and light chains of first heterodimer and the heavy and light chains of the second heterodimer. Exemplary nucleic acid sequences encoding CDRs, variable regions, and heavy and light chains that bind to DLL3 and human CD3 are set forth in Sequence Tables 15 and 15A below.

The disclosure further provides a vector comprising one or more nucleic acid sequences encoding the heteromultimer, or components thereof (e.g., the first heterodimer and/or the second heterodimer). The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994))

The heteromultimers described herein can be produced by recombinant DNA methodologies known in the art. “Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant. Thus, the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

In some embodiments, the heteromultimer may be produced by a process in which a host cell (e.g., Chinese hamster ovary cells) comprising one or more nucleic acid sequences encoding the first heterodimer (which binds DLL3) and/or the second heterodimer (which binds CD3) described herein are cultured under conditions allowing the expression of the heteromultimer, and the expressed heteromultimer is then recovered from the cell culture.

### Methods of Inhibiting DLL3-Expressing Cancer

The disclosure also provides a method of inhibiting growth of DLL3-expressing cancer cells, which comprises contacting a population of DLL3-expressing cancer cells and CD3-expressing T cells with an effective amount of the heteromultimer, or a composition comprising the heteromultimer, as described above. In some embodiments, the population of DLL3-expressing cancer cells and CD3-expressing T cells can be contacted with the above-described heteromultimer, or composition comprising same, ex vivo, in vivo, or in vitro. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context.

For any of the methods of treating cancer cells described herein, the cancer cells desirably express DLL3 on the cell surface. In some aspects, cell surface expression of DLL3 protein may be determined by immunohistochemistry (IHC) or positron emission tomography (PET). For example, at least 5% (e.g., 5%, 10%, or 20%) of the cells of a cancer may be positive for DLL3 as determined by IHC. Any suitable IHC assay for determining DLL3 protein expression may be used in connection with the present disclosure. Desirably, the IHC assay is approved by a regulatory agency, such as the U.S. Food and Drug Association (FDA) or the European Medicines Agency (EMA)). DLL3-specific IHC assays, and components thereof, are known in the art and commercially available from a variety of sources. For example, DLL3 expression in cancer or tumor cells may be detected using the VENTANA® DLL3 (SP347) Assay (Roche Diagnostics, GmbH, Mannheim, Germany). Other anti-DLL3 antibodies that may be used to detect DLL3 expression in an IHC assay include, but are not limited to, NBP2-24669 (Novus Biological, Littleton, CA); PA5-26336 (Thermo Fisher Scientific, Waltham, MA); and ab229902 (Abcam, Cambridge, MA). Exemplary PET assays for determining DLL protein expression are described in Chou et al., Cancer Res (2023) 83 (2): 301-315; Sharma et al., Cancer Res. 2017 Jul. 15; 77 (14): 3931-3941. Doi: 10.1158/0008-5472.CAN-17-0299; and Poirier, J. T., Journal of Thoracic Oncology Vol. 15 No. 2S (2020). Expression of DLL3 mRNA can be determined using methods known in the art, such as, for example, flow cytometry-based methods polymerase chain reaction (PCR) analysis, sequencing analysis (e.g., RNA sequencing), electrophoretic analysis, restriction fragment length polymorphism (RFLP) analysis, Northern blot analysis, quantitative PCR, reverse-transcriptase-PCR analysis (RT-PCR), and the like.

In exemplary aspects of the methods described herein, the heteromultimer also binds to human CD3 expressed on the surface of a T cell. As discussed above, CD3 associates with T cell receptors to form the T cell receptor complex, which leads to the generation of an activation signal in T lymphocytes. In some embodiments, the second heterodimer comprises a heavy chain polypeptide and a light chain polypeptide comprising a CD3-binding amino acid sequence denoted “I2E” or “I2E2,” the sequences of which are set forth in the Sequence Tables below.

Cancer cells of any suitable type may be contacted with the heteromultimer or composition described herein. In certain embodiments, the cancer is a neuroendocrine cancer. Neuroendocrine cancers or neoplasms (NECs or NENs) are a relatively rare and heterogeneous tumor type, comprising ˜2% of all malignancies, with a prevalence of <200,000 in the United States (Oronsky et al., Neoplasia, 19 (12): 991-1002 (2017)). The term “neuroendocrine” is applied to widely dispersed cells with properties similar to neural cells, such as the presence of dense core granules (DCGs4) that are similar to DCGs present in serotonergic neurons, which store monoamines, and “endocrine” properties, such as the synthesis and secretion of these monoamines. The neuroendocrine (NE) system includes endocrine glands, such as the pituitary, the parathyroids, and the NE adrenal, as well as endocrine islet tissue embedded within glandular tissue (thyroid or pancreatic) and scattered cells in the exocrine parenchyma, such as endocrine cells of the digestive and respiratory tracts, which belong to what is known as the diffuse endocrine system. Most neuroendocrine tumors occur in the lungs, appendix, small intestine, rectum and pancreas. Neuroendocrine cancers include, but are not limited to, small cell lung cancer (SCLC), neuroendocrine prostate cancer (NEPC), and neuroblastoma.

In some embodiments, the tumor or cancer is lung cancer such as SCLC or non-small cell lung cancer (NSCLC), glioma, glioblastoma, melanoma, prostate cancer such as NEPC, neuroendocrine pancreatic cancer, hepatoblastoma, large cell pulmonary neuroendocrine cancer, pancreatic neuroendocrine cancer, bladder neuroendocrine cancer, gastric neuroendocrine cancer, adrenal exocrine tumors, Merkel cell carcinoma, neuroblastoma, head and neck carcinoid or neuroendocrine cancer, head and neck paraganglioma, or cervical small cell neuroendocrine cancer. In some embodiments, the tumor or cancer is neuroendocrine prostate cancer.

In exemplary aspects, the cancer is a histologically or cytologically confirmed SCLC. Optionally, the SCLC is measurable by modified Response Criteria in Solid Tumors (RECIST) 1.1, wherein measurable lesions include (a) non-nodal lesions with clear borders that can be measured accurately and serially in one dimension in the axial plane (longest diameter ≥10 mm measured by magnetic resonance imaging/computed tomography (MRI/CT) with scan slice thickness ≤5 mm) and/or (b) nodal lesions with the longest diameter perpendicular to the long axis (short axis) ≥15 mm on MRI/CT, and/or exclude simple cysts, pleural/pericardial effusions and ascites.

In embodiments where the cancer cells are in vivo, the disclosure provides a method of treating a DLL3-expressing cancer in a subject in need thereof, which comprises administering to the subject an effective amount of the heteromultimer described herein, or a composition comprising the heteromultimer. The present disclosure also provides use of the above-described heteromultimer in the manufacture of a medicament for the treatment of a DLL3-expressing cancer. As discussed above, the cancer may be a neuroendocrine cancer, including but not limited to, SCLC, NEPC, or neuroblastoma.

The term “treatment” includes prophylactic and/or therapeutic treatments. If it is administered prior to clinical manifestation of a condition, the treatment is considered prophylactic. Therapeutic treatment includes, e.g., ameliorating or reducing the severity of a disease, or shortening the length of the disease. Also, the term “treat,” as well as words related thereto, does not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods may delay the onset or recurrence of the cancer by at least about 30 days, two months, 4 months, 6 months, 1 year, 2 years, 4 years, or more. In exemplary aspects, treatment may encompass increasing the survival of the subject. In various aspects, the treatment provided by the methods of the present disclosure includes a therapeutic response as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer.

Therapeutic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the disclosure.

As discussed above, the disclosed heteromultimer, or a composition comprising the heteromultimer, may be administered to a subject using standard administration techniques, including oral, intravenous, intraperitoneal, subcutaneous, pulmonary, transdermal, subcutaneous, intramuscular, intranasal, buccal, sublingual, or suppository administration. The desired dosage can be delivered by a single bolus administration of the composition, by multiple bolus administrations of the composition, by continuous infusion administration of the composition, or any combination of the foregoing administration methods.

In some embodiments of the methods disclosed herein, the above-described heteromultimer or composition may be administered alone (i.e., as a “monotherapy”), or in combination with at least one additional therapeutic agent to achieve a desired biological effect in a subject. In exemplary aspects, the at least one additional therapeutic agent may be a cancer treatment. The choice of cancer treatment used in combination with the disclosed method will depend on a variety of factors, including the cancer/tumor type, stage and/or grade of the tumor or cancer, the subject's age, etc. Suitable cancer treatments that may be employed include, but are not limited to, surgery, chemotherapy, radiation therapy, targeted therapy, immunotherapy, hormone therapy, and stem cell transplantation.

In some aspects, the at least one additional therapeutic agent may be a chemotherapeutic agent. “Chemotherapeutic agents,” also referred to as antineoplastic agents, include compounds useful for the treatment of cancer. Chemotherapeutic agents can be classified according to their mechanism of action and can be further divided into subgroups within each class. Exemplary classes of chemotherapeutic agents include alkylating agents, antimetabolites, topoisomerase inhibitors, anti-tumor antibiotics, mitotic inhibitors, and protein kinase inhibitors. Alkylating agents include subgroups such as oxazaphosphorines, nitrogen mustards, imidazotetrazines, nitrosoureas, alkyl sulfonate, hydrazines, and platinum-based agents. Platinum-based agents include cisplatin, carboplatin, and oxaliplatin. Topoisomerase inhibitors include topoisomerase I inhibitors and topoisomerase II inhibitors. Mitotic inhibitors include vinca alkaloids, taxanes, and nontaxane microtubule inhibitors. Anti-tumor antibiotics include bleomycin, actinomycin D (dactinomycin), and mitomycin.

In certain embodiments, the chemotherapeutic agent that can be used in the method disclosed herein is an alkylating agent. In exemplary embodiments, the alkylating agent may be a platinum-based agent such as cisplatin, carboplatin, or oxaliplatin. In certain embodiments, the alkylating agent is lurbinectedin (ZEPZELCA™). In other embodiments, the chemotherapeutic agent may be a topoisomerase inhibitor such as a topoisomerase II inhibitor (e.g., etoposide). In certain embodiments, the chemotherapeutic agent that can be used in the methods disclosed herein includes a platinum-based agent (cisplatin, carboplatin, or oxaliplatin), a topoisomerase II inhibitor (etoposide) or a combination of a platinum-based agent and a topoisomerase II inhibitor.

In other aspects, the at least one additional therapeutic may be a targeted cancer therapy (also referred to as “precision oncology therapy”). Exemplary targeted cancer therapies include, but are not limited to, protein kinase inhibitors (e.g., BCR-ABL and c-KIT tyrosine kinase inhibitors, EGFR tyrosine kinase inhibitors, ALK tyrosine kinase inhibitors, V600E mutated-BRAF oncogene inhibitors, MEK inhibitors, Bruton kinase inhibitors, Janus kinase inhibitors, and CDK inhibitors).

In other exemplary aspects, the at least one additional therapeutic agent may be a programmed cell death 1 (PD-1)/programmed cell death ligand 1 (PD-L1) antagonist. PD-1, also known as CD279, SLEB2, and hSLE1, is a transmembrane protein expressed on activated T cells, natural killer (NK) cells, B lymphocytes, macrophages, dendritic cells (DCs) and monocytes. Notably, PD-1 is highly expressed on tumor-specific T cells (Han et al., Am J Cancer Res 10 (3): 727-742 (2020)). PD-1 binds to B7 protein family members, programmed death (PD) Ligand 1 (PD-L1; also referred to as CD274 and B7-H1) and PD Ligand 2 (also known as PD-L2, CD273, and B7-DC). PD-L1 is constitutively expressed on T and B cells, macrophages, and dendritic cells, whereas PD-L2 expression is typically restricted to activated DCs and macrophages (Xing et al., Oncoimmunology 7 (3): e1356144 (2017) (doi: 10.1080/2162402X.2017.1356144)). PD-1 inhibits both adaptive and innate immune responses.

The PD-1/PD-L1 axis is involved in the suppression of T cell immune responses in cancer. Antagonists of this pathway have been clinically validated across a number of solid tumor indications. Antagonistic anti-PD-1 antibodies and anti-PD-L1 antibodies have been approved by the U.S. Food and Drug Administration (FDA) for the treatment of various cancers. In some embodiments, agents targeting PD-1 (e.g., a PD-1 antagonist or inhibitor) and/or PD-L1 (e.g., a PD-L1 antagonist or inhibitor) can be used in methods disclosed herein to treat DLL3-expressing cancers. Exemplary agents targeting PD-1 include, but are not limited to, anti-PD-1 antibodies such as nivolumab, pembrolizumab, and cemiplimab. Exemplary agents targeting PD-L1 include, but are not limited to, anti-PD-L1 antibodies such as atezolizumab, avelumab, and durvalumab.

In certain embodiments, the anti-PD-L1 antibody is atezolizumab (International Nonproprietary Name for Pharmaceutical Substances (INN), WHO Drug Information, Vol. 29, No. 3, 2015, Recommended INN: List 74). Atezolizumab is a humanized PD-L1 blocking antibody. It is an immunoglobulin G1-kappa, anti-[Homo sapiens CD274 (programmed death ligand 1, PDL1, PD-L1, B7 homolog 1, B7H1)], humanized monoclonal antibody; gamma1 heavy chain (1-448) [humanized VH (Homo sapiens IGHV3-23*04 (86.70%)-(IGHD)-IGHJ4*01) [8.8.11] (1-118)-Homo sapiens IGHG1*03 (CH1 R120>K (215) (119-216), hinge (217-231), CH2 N84.4>A (298) (232-341), CH3 (342-446), CHS (447-448)) (119-448)], (221-214′)-disulfide with kappa light chain (1′-214′) [humanized V-KAPPA (Homo sapiens IGKV1-5*01 (87.90%)-IGKJ1*01) [6.3.9] (1′-107′)-Homo sapiens IGKC*01 (108′-214′)]; dimer (227-227″: 230-230″)-bisdisulfide. Atezolizumab is available commercially, e.g., it is marketed as TECENTRIQ®.

In certain embodiments, the anti-PD-L1 antibody is avelumab (International Nonproprietary Name for Pharmaceutical Substances (INN), WHO Drug Information Vol. 30, No. 1, 2016, Recommended INN: List 75). Averlumab is a PD-L1 blocking monoclonal antibody produced in CHO cells. It is an immunoglobulin G1-lambda1, anti-[Homo sapiens CD274 (programmed death ligand 1, PDL1, PD-L1, B7 homolog 1, B7H1)], Homo sapiens monoclonal antibody; gamma1 heavy chain (1-450) [Homo sapiens VH (IGHV3-23*01 (90.80%)-(IGHD)-IGHJ4*01) [8.8.13] (1-120)-IGHG1*01, Gm17,1 (CH1 (121-218), hinge (219-233), CH2 (234-343), CH3 (344-448), CHS (449-450) (121-450)], (223-215′)-disulfide with lambda1 light chain (1′-216′) [Homo sapiens V-LAMBDA (IGLV2-14*01 (99.00%)-IGLJ1*01) [9.3.10] (1′-110′)-IGLC1*02 (111′-216′)]; dimer (229-229″: 232-232″)-bisdisulfide. Avelumab is commercially available, e.g., it is marketed as BAVENCIO®.

In certain embodiments, the anti-PD-L1 antibody is durvalumab (International Nonproprietary Name for Pharmaceutical Substances (INN), WHO Drug Information, Vol. 29, No. 3, 2015, Recommended INN: List 74). Durvalumab is a PD-L1 blocking monoclonal antibody produced in CHO cells. It is an immunoglobulin G1-kappa, anti-[Homo sapiens CD274 (programmed death ligand 1, PDL1, PD-L1, B7 homolog 1, B7H1)], Homo sapiens monoclonal antibody; gamma1 heavy chain (1-451) [Homo sapiens VH (IGHV3-7*01 (99.00%)-(IGHD)-IGHJ4*01) [8.8.14] (1-121)-IGHG1*03 (CH1 (122-219), hinge (220-234), CH2 (235-344) L1.3>F (238), L1.2>E (239), P116>S (335), CH3 (345-449), CHS (450-451)) (122-451)], (224-215′)-disulfide with kappa light chain (1′-215′) [Homo sapiens V-KAPPA (IGKV3-20*01 (96.90%)-IGKJ1*01) [7.3.9] (1′-108′)-IGKC*01 (109′-215′)]; dimer (230-230″: 233-233″)-bisdisulfide. Durvalumab is commercially available, e.g., it is marketed as IMFINZI®.

In various aspects of the present disclosure, the subject is a human. In exemplary aspects, the human subject has SCLC, optionally, a histologically or cytologically confirmed SCLC. In various aspects, the human is male or female and/or greater than or equal to 18 years of age with a SCLC. In exemplary aspects, the human subject has relapsed/refractory (RR) SCLC, optionally, which progressed or recurred following at least one platinum-based chemotherapy with or without a PD-L1 inhibitor. In exemplary aspects, the human subject has extensive stage SCLC (ES-SCLC), optionally, histologically or cytologically confirmed ES SCLC. In exemplary aspects, the human subject has ES-SCLC and has received no prior systemic treatment for ES-SCLC. In exemplary aspects, the human subject has an Eastern Cooperative Oncology Group (ECOG) performance status of 0-1 (Oken et al., Am J Clin Oncol 5:649-655 (1982)).

The following examples further illustrates the invention but, of course, should not be construed as in any way limiting its scope.

### Example 1

This example describes the generation of heteromultimers encompassed by the present disclosure.

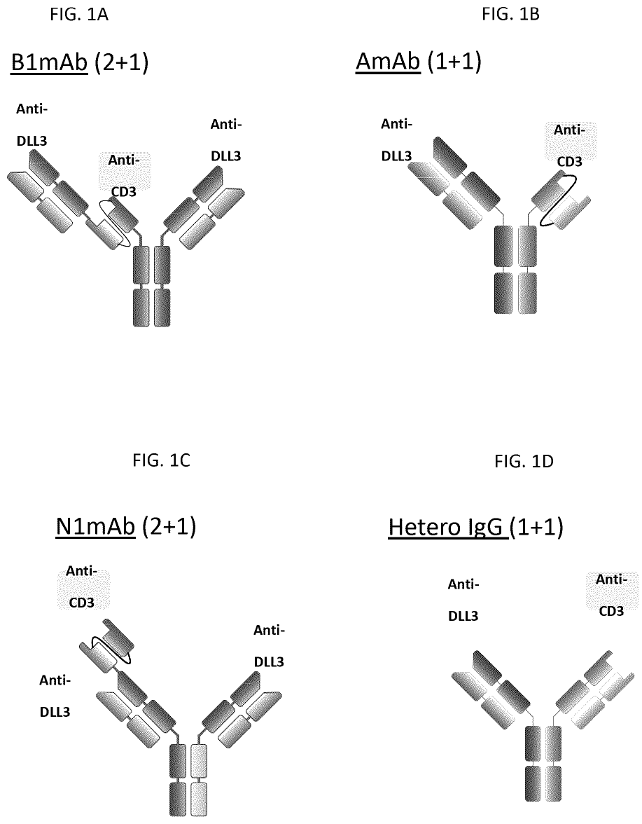

DLL3-targeted multi-chain T cell engager molecules (referred to as “mcTCE”) were generated using three anti-DLL3 binding domains against diverse epitopes, and a high-affinity anti-CD3 binding domain. HeteroIgG, AmAb (“Amgen monoclonal antibody”), NmAb (1 scFv at the N-terminus of 1 Fab arm), and BmAb (1 scFv between 1 Fab arm and the Fc part) formats were generated (see FIGS. 1A-1D) and evaluated for expression, purification, binding affinity, cell-based activity, potential for immunogenicity, pharmacokinetic profile and physical and chemical stability.

In particular, on-cell binding affinity for DLL3 was evaluated using Chinese Hamster Ovary (CHO) cells engineered to stably express either human or cynomolgus monkey DLL3. The engineered CHO cells were incubated with DLL3 multichain TCE proteins comprising two different DLL3-binders (“DLL3_1” or “DLL3_2”) for 4 hours at 4° C. in buffer (phosphate buffered saline with 1% fetal bovine serum). After incubation, the cells were washed in buffer, and then bound DLL3 multichain proteins were detected by incubation with a fluorescently labeled antibody directed to the fragment crystallizable (Fc) region of the proteins. Bound proteins were analyzed by flow cytometry and the EC50 values were calculated from non-linear regression analysis using GraphPad Prism. The results are shown in Table 1.

TABLE 1
DLL3 Binding Affinity

| | | Human | Cynomolgus |
|---|---|---|---|
| | | DLL3 EC₅₀ | monkey DLL3 |
| Format | Construct | (nM) | EC₅₀ (nM) |
| Hetero IgG | DLL3_1 hetero IgG (SEFL2)(YTE) | 0.120 ± 0.050 | 0.110 ± 0.030 |
| AmAb | DLL3_1 AmAb (YTE) | 0.060 ± 0.013 | 0.075 ± 0.016 |
| N1 mAb | DLL3_1 N1 mAb (YTE) | 0.019 ± 0.007 | 0.023 ± 0.011 |
| N1 mAb | DLL3_1 N1 mAb (W139C, A51C)(YTE) | 0.013 ± 0.005 | 0.016 ± 0.008 |
| B1 mAb | DLL3_1 B1 mAb (YTE) | 0.025 ± 0.011 | 0.031 ± 0.014 |
| B1mAb | DLL3_1 B1 mAb (W139C, A51C) (YTE) | 0.021 ± 0.007 | 0.023 ± 0.008 |
| Hetero IgG | DLL3_2 hetero IgG (SEFL2)(YTE) | 0.035 ± 0.015 | 0.040 ± 0.016 |
| N1 mAb | DLL3_2 N1 mAb (W139C, A51C)(YTE) | 0.022 ± 0.006 | 0.013 ± 0.005 |
| B1 mAb | DLL3_2 B1 mAb (W139C, A51C)(YTE) | 0.021 ± 0.004 | 0.019 ± 0.008 |

CD3 binding affinity was evaluated using surface plasmon resonance. A CD3E fusion protein (CD3ε (aa 1-27)-chicken albumin) was immobilized on CM5 Sensor Chips in the presence of a sodium acetate buffer (pH 4.5). The DLL3 multichain TCE proteins described above diluted in HBS-EP buffer were applied at different concentrations to determine the molecule association rates with CD3ε. HBP-EP buffer alone was then added to allow analysis of the molecule dissociation rates. Binding affinity calculations were done using BiaEval software, and the results are shown in Table 2.

TABLE 2
CD3 Binding Affinity

| | | Human | Cynomolgus |
|---|---|---|---|
| | | CD3 K_{D} | monkey CD3 |
| Format | Construct | (nM) | K_{D} (nM) |
| Hetero IgG | DLL3_1 hetero IgG (SEFL2)(YTE) | 9.45 ± 0.04 | 6.42 ± 0.04 |
| AmAb | DLL3_1 AmAb (YTE) | 13.00 ± 2.97 | 8.67 ± 2.02 |
| N1 mAb | DLL3_1 N1 mAb (YTE) | 18.15 ± 1.06 | 12.50 ± 0.57 |
| N1 mAb | DLL3_1 N1 mAb (W139C, A51C)(YTE) | 8.00 ± 0.06 | 5.17 ± 0.05 |
| B1 mAb | DLL3_1 B1 mAb (YTE) | 28.35 ± 0.07 | 18.80 ± 0.00 |
| B1mAb | DLL3_1 B1 mAb (W139C, A51C) (YTE) | 13.75 ± 0.35 | 8.79 ± 0.11 |
| Hetero IgG | DLL3_2 hetero IgG (SEFL2)(YTE) | 7.84 ± 1.39 | 5.37 ± 1.03 |
| N1 mAb | DLL3_2 N1 mAb (W139C, A51C)(YTE) | 8.26 ± 0.21 | 5.49 ± 0.11 |
| B1 mAb | DLL3_2 B1 mAb (W139C, A51C)(YTE) | 10.50 ± 0.14 | 6.64 ± 0.17 |

Cytotoxicity was assessed using a T cell dependent cellular cytotoxicity (TDCC) assay. To this end, unstimulated human peripheral blood mononuclear cells (PBMC; CD14-/CD56-) were co-cultured with target cells (CHO cells (untransfected, or stably expressing human DLL3), or small cell lung cancer cell lines SHP-77 or NCI-H82) at an effector to target cell (E: T) ratio of 10:1 and incubated with a concentration range of DLL3 multichain TCE proteins for 48 hours. Cynomolgus monkey T cell line LnPx4119 was co-cultured at a 10:1 ratio with CHO cells or CHO cells expressing cynomolgus monkey DLL3 and incubated with a concentration range of DLL3 multichain TCE molecules for 48 hours. T cell-dependent cellular cytotoxicity was measured with a flow cytometry-based assay where target cells were pre-labeled with DiO labeling dye (marking live cells) and stained with propidium iodide (marking dead cells) after the 48 hour incubation. Data were analyzed by non-linear regression in GraphPad Prism and are shown in Table 3. The DLL3_1 and DLL3_2 multimers in heteroIgG format showed favorable cell potency.

TABLE 3
Cellular Cytotoxicity

| | | CHO- | CHO-cyno | SHP-77 | DMS-79 |
|---|---|---|---|---|---|
| | | huDLL3 EC₅₀ | DLL3 EC₅₀ | EC₅₀ | EC₅₀ |
| Format | Construct | (pM) | (pM) | (pM) | (pM) |
| Hetero IgG | DLL3_1 hetero IgG (SEFL2)(YTE) | 230 | 54.6 | 186 | 18.0 |
| AmAb | DLL3_1 AmAb (YTE) | 241 | 10.5 | 266 | 118 |
| N1 mAb | DLL3_1 N1 mAb (YTE) | 64.0 | 30.5 | ND | ND |
| N1 mAb | DLL3_1 N1 mAb (W139C, | 80.0 | 33.8 | ND | ND |
| | A51C)(YTE) | | | | |
| B1 mAb | DLL3_1 B1 mAb (YTE) | 48.8 | 4.7 | 86.5 | 42.0 |
| B1mAb | DLL3_1 B1 mAb (W139C, A51C) | 198 | 11.2 | 202 | 94.0 |
| | (YTE) | | | | |
| Hetero IgG | DLL3_2 hetero IgG (SEFL2)(YTE) | 100 | 22.7 | 12.2 | 2.8 |
| N1 mAb | DLL3_2 N1 mAb (W139C, | 221 | 314 | 459 | 71.5 |
| | A51C)(YTE) | | | | |
| B1 mAb | DLL3_2 B1 mAb (W139C, | 284 | 36.7 | 330 | 56.7 |
| | A51C)(YTE) | | | | |

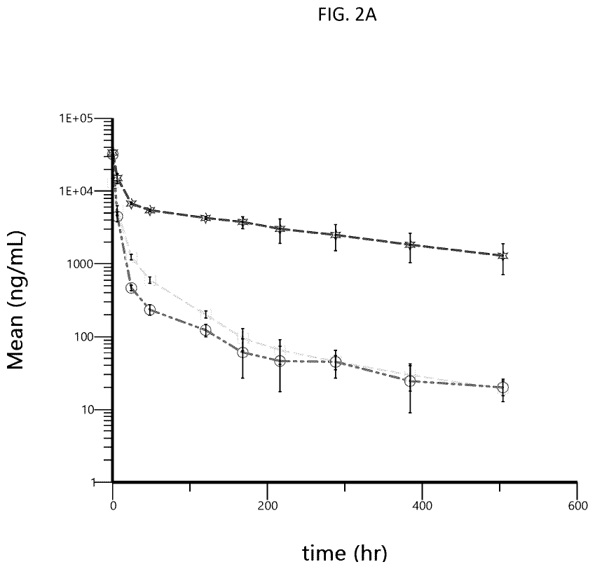

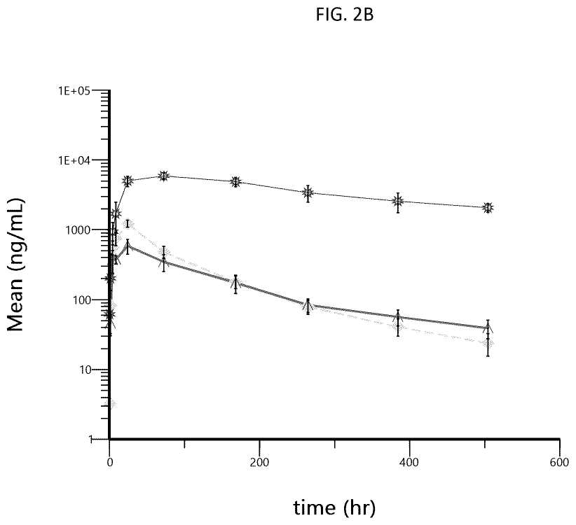

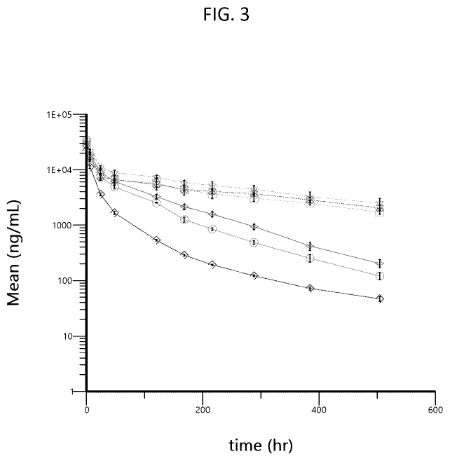

The DLL3_1 and DLL3_2 heteroIgG molecules also showed an antibody-like pharmacokinetic profile in a transgenic mouse model (9.7-15.2 days; FIGS. 2A-2B and FIG. 3), and acceptable physical and chemical stability. To mitigate risk associated with potential thermal instability at residue N103 in the CD3 CDR, versions of the DLL3_1 heteroIgG and DLL3_2 heteroIgG containing another CD3 binding domain were tested and shown to have improved stability.

### Example 2

This example describes analysis of the productivity of a DLL3-targeted heteroIgG molecule encompassed by the disclosure.

A DLL3-specific heteroIgG molecule was generated in accordance with the methods described herein. The heteroIgG molecule (also denoted “DLL3_2”) contains a DLL3-binding heterodimer comprising a heavy chain amino acid sequence of SEQ ID NO: 54 and a light amino acid sequence of SEQ ID NO: 55, and CD3-binding heterodimer comprising a heavy chain amino acid sequence of SEQ ID NO: 56 and a light chain amino acid sequence of SEQ ID NO: 57.

Briefly, cells were cultivated in a stirred tank bioreactor in a 15-day perfusion system using an alternating tangential flow (ATF) filtration system (Repligen, Waltham MA, USA) connected to polysulfone filters (Cytiva, Westborough, MA, USA), and a proprietary chemically defined medium. On day 12, the ATF filter was switched from a 30 kDa retentive membrane to a 750 kDa membrane which allows product to pass through the membrane while cells are still retained in the bioreactor. The accumulated harvested cell culture fluid (HCCF) represents the collected product (XMF harvest) over the 3 day of perfusion process with the 750 kDa membrane. The HCCF product (in g) was calculated by multiplying the HCCF titer (g/L) with the XMF harvest volume (L).

The equivalent or Final Total Bioreactor titer is a calculated value that represents the total productivity of the reactor, adjusted to the reactor volume. This number allows comparison across projects and scale. It was calculated by adding the final reactor product (g of product remaining in the bioreactor after perfusion and adjusted by the packed cell volume) with the HCCF product (in g) and dividing by the reactor volume. This calculated combined titer is equivalent to a titer measured in a conventional fed-batch bioreactor process.

### Titer Assay

To analyze the concentration of a CHO-expressed recombinant hetero IgG protein in conditioned media like XMF harvest an affinity Protein A Ultra Performance Liquid Chromatography (UPLC) was used.

In brief it utilizes affinity chromatography where Protein A is immobilized on a column support. At neutral pH, the CHO-expressed hetero-IgG binds to the Protein A through the Fc region while host-cell proteins (HCPs), conditioned media components, and buffer are not retained and are eluted from the column in the flow-through. Captured mAbs and CHO-expressed FC-Fusion proteins are eluted at acidic pH and detected by UV absorbance at 280 nm. A calibration curve is derived from a mAb or CHO expressed Fc-Fusion protein standard and the corresponding peak areas using linear regression analysis.

### Yield Calculations

The harvest yield (in %) or XMF harvest yield was calculated by dividing the HCCF product amount (in g) by the combined total product (in g).

The purification yield is the sum of all steps yields in the purification workflow. Here, the purification yield represents the sum of the step yields from the protein-A capture, cation exchange chromatography (CEX), a multi module step (MMC), viral filtration, and a final ultrafiltration/diafiltration (UF/DF) concentration and formulation step.

The overall yield is the combination of the XMF harvest and the purification yield. It represents the ratio of the final drug substance (DS) (in g) over the combined total product (in g).

The final expected productivity (in g DS/L bioreactor volume) it the product of the final total bioreactor titer (g/L) with the overall yield (in %). The productivity, titer, and yields of DLL3_2 single cell clones as compared to AMG 757 (tarlatamab) are shown in Table 4. As discussed herein, AMG 757 is DLL3-specific a half-life-extended (HLE) BiTE® molecule developed for the treatment of SCLC. Surprisingly, the drug substance per L bioreactor volume yield of DLL3_2 (2.7 g/L) was 270% more than AMG 757.

TABLE 4

| | AMG 757 | DLL3_2 |
|---|---|---|
| Final Total Titer Bioreactor g/L* | 3.1 | 7.3 |
| Harvest yield (%) | 67.3 | 62.0 |
| Purification yield (%) | 50.1 | 60.0 |
| Overall yield (%) | 33.7* | 37.0 |
| (Harvest/Purification Yield) | | |
| Expected Productivity | 1.0 | 2.7 |
| g DS per L Bioreactor (g DS/L) | | |

*3-step purification

### Example 3

This example describes the molecular characterization of a DLL3-targeted heteroIgG molecule encompassed by the disclosure.

Biochemical, biophysical, and biological characterization of DLL3 #2 was conducted at various stages of downstream production to provide a comprehensive understanding of its structural and functional properties and to enable an assessment of attributes (e.g., aggregation, high molecular weight (HMW) species, and charge variants) that may affect binding and potency. For example, visual particles present in a platform DLL3 #2 formulation were assessed by visual inspection; high molecular weight species were assessed by size exclusion chromatography (SEC); low molecular weight species were assessed by rCE-SDS (reduced capillary electrophoresis-sodium dodecyl sulfate); charge variants were assessed by CEX-UPLC (cation exchange ultra-performance liquid chromatography); chemical modifications to DLL3 #2 were assessed by peptide mapping; subvisible particles were assessed by HAIC (high accuracy liquid particle counter) and/or BMI (backgrounded membrane imaging); and protein concentration/recovery was determined by SPR (surface plasmon resonance). An IV compatibility test and agitation assessment also were performed.

CEX-UPLC assessment showed slightly increased acidic and basic peak levels after a second Photo Stress (P2), and increased acidic and basic peak levels after storage of a platform formulation of DLL3 #2 at 40° C. An increase in HMWs was observed after P2 and after storage at 40° C. There was no increase of HMWs after storage at −20° C. and after freeze-thaw.

rCE-SDS analysis revealed increased polypeptide clipping after 40° C. storage; however the change in LMWs (<5%) after 4 weeks storage at 40° C. was within a predetermined quality profile.

Titer, HMW species, LMW species, and charge variants of amplified pools of DLL3_2 were compared to other DLL3-specific heteroIgG molecules (A, B, and C), as well as representative BiTE®-HLE molecules. Table 5 shows that the average titer of the amplified DLL3_2 pools was 2-3 times higher than for pools of BiTE®-HLE molecules, and the individual DLL3 TCE heteroIgG pools have significantly improved titer and product quality as compared to the BiTE-HLE pools. Thus, DLL3-specific heteromultimers encompassed by the disclosure, including DLL3_2, have advantageous manufacturing properties as compared to traditional BiTE® molecules.

TABLE 5

| | | | SEC | | rCE | | | |
|---|---|---|---|---|---|---|---|---|
| Molecule | | Titer | | HMW | LMW | CEX | | |
| Description | | (g/L) | Main (%) | (%) | (%) | Basic (%) | Acidic (%) | Main (%) |
| DLL3 | A | 1.6 ± 0.7 | 60.1 ± 10.4 | 13.3 ± 3.7 | 3.1 ± 1.2 | 13.6 ± 4.7¹ | 17.6 ± 7.6² | 63.4 ± 4.6³ |
| TCE | B | 2.1 ± 0.9 | 76.5 ± 6.4 | 7.7 ± 0.9 | 0.9 ± 0.9 | 15.7 ± 1.4⁴ | 24.6 ± 20.8⁴ | 59.7 ± 22.3⁴ |
| hetero | DLL3_2 | 1.8 ± 0.6 | 70.2 ± 5.5 | 10.3 ± 2.5 | 2.7 ± 1.4 | 22.2 ± 7.1⁵ | 24.6 ± 14.1⁵ | 53.3 ± 14.0⁵ |
| IgG | C | 1.4 ± 0.6 | 77.3 ± 5.2 | 6.7 ± 2.7 | 1.2 ± 1.4 | 13.5 ± 14.4⁶ | 26.1 ± 10.8 | 55.4 ± 7.5 |
| BiTE | Min/Max | 0.2-0.7 | >70% | <30% | ~5% | 25-35% | 5-20% | 45-60% |
| HLE | Average | 0.42 ± 0.13 | 76.4 ± 7.4 | 23.1 ± 6.9 | 5.1 ± 0.8 | 27.7 ± 4.6 | 14.6 ± 7.4 | 51.4 ± 9.4 |
| (pools) | | | | | | | | |

### Example 4

This example describes an exploratory single-dose pharmacokinetic and local tolerance study of a DLL3-targeted heteroIgG molecule in the cynomolgus monkey.

Quantitation of DLL3_2 (described above) in cynomolgus monkey serum was performed using three electrochemiluminescent immunoassays with 1) a biotinylated anti-CD3 monoclonal antibody (MAb) as the capture reagent and a ruthenylated mouse anti-human IgG Fc MAb as the detection reagent (assay-1), 2) biotinylated human DLL3 (R&D Systems, Cat #9749-DL) as the capture reagent and a ruthenylated mouse anti-human IgG Fc MAb as the detection reagent (assay-2), and 3) biotinylated mouse anti-human IgG Fc Mab as the capture reagent and a ruthenylated mouse anti-human IgG Fc MAb as the detection reagent (assay-3). Analyte serum concentrations were interpolated from a standard curve using the corresponding analyte. The lower limit of quantitation (LLOQ) for the assay in serum was 0.61 ng/ml, while the upper limit of quantitation (ULOQ) for the assay in serum was 10000 ng/ml in all formats.

Toxicokinetic analysis was performed, and data for TK analysis was extracted from the non-GLP Watson database to PHOENIX®. Noncompartmental analysis (NCA) was performed on the individual serum concentration-nominal time data from 0 to 168 hours post dose using PHOENIX® WINNONLIN® (version 6.4). The following PK parameters were estimated: Cₘₐₓ (maximum concentration in serum), AUC₀₋₃₃₆ (area under the concentration-time curve from time 0 to 336 hours post dose, estimated by the linear trapezoidal method).

All the three assay formats yielded similar serum concentrations of the administered molecule, indicating no major clipping of the molecules in the serum during due course of the experiment. Rapid decrease of serum concentration of the administered molecule was observed post 336 hrs. This decrease in serum concentration could be attributed to possible generation of anti-drug antibodies (ADAs). These ADAs could have resulted in faster clearance of administered molecules or might have interfered with either capture or detection reagents inhibiting the detection of administered molecules.

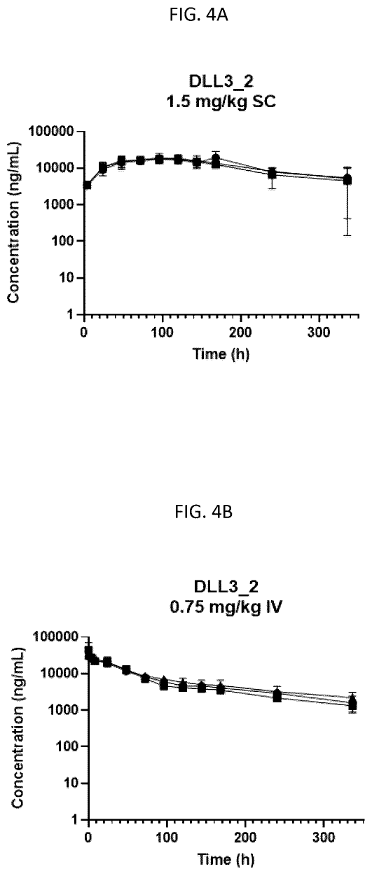

DLL3_2 exhibited a good serum exposure when administered either subcutaneously or intravenously, as shown in FIGS. 4A and 4B. Bioavailability [(mean AUC_{SC}/Dose_{SC})/(mean AUC_{IV}/Dose_{IV})] of DLL3_2 in cynomolgus monkey was 93.6%, indicating good release of DLL3_2 to circulation from the subcutaneous administration site.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 6
SEQUENCE TABLES
huCD3 CDR Sequences

| | Seq ID | | Seq ID | | Seq ID | |
|---|---|---|---|---|---|---|
| Name | No. | CDR1 | No. | CDR2 | No. | CDR3 |
| DLL3_8-A7 | 7 | KYAIN | 8 | RIRSKYNNYATYYADAVKD | 19 | AGNFGSSYISYWAY |
| KDDD_huCD3_29019_EKK_heteroIgG | | | | | | |
| (1zSEFL2v503YTE) HC E huCD3 HV | | | | | | |
| DLL3_8-A7 | 10 | GSSTGAVTSGNYPN | 11 | GTKFLAP | 12 | VLWYSNRWV |
| KDDD_huCD3_29019_EKK_heteroIgG | | | | | | |
| (1zSEFL2v503YTE)_LC_K huCD3 LV | | | | | | |
| huDLL3(40861)_huCD3(29019(VH: | 7 | KYAIN | 8 | RIRSKYNNYATYYADAVKD | 9 | AENIGTSYISYWAY |
| G110E_F112I_S114T))_heteroIgG4PE- | | | | | | |
| FALA-KiH HC E huCD3 HV | | | | | | |
| huDLL3(40861)_huCD3(29019(VH: | 10 | GSSTGAVTSGNYPN | 11 | GTKFLAP | 12 | VLWYSNRWV |
| G110E_F112I_S114T))_heteroIgG4PE- | | | | | | |
| FALA-KiH_LC_K huCD3 LV | | | | | | |
| DLL3_8- | 7 | KYAIN | 8 | RIRSKYNNYATYYADAVKD | 9 | AENIGTSYISYWAY |
| A7_KDDD_huCD3_I2E2_29019_EKK_ | | | | | | |
| heteroIgG(1zSEFL2v503YTE)_ | | | | | | |
| HC_E huCD3 HV | | | | | | |
| DLL3_8- | 10 | GSSTGAVTSGNYPN | 11 | GTKFLAP | 12 | VLWYSNRWV |
| A7_KDDD_huCD3_I2E2_ 29019_EKK_ | | | | | | |
| heteroIgG(1zSEFL2v503YTE) LC K | | | | | | |
| huCD3 LV | | | | | | |
| DLL3_0- | 7 | KYAIN | 8 | RIRSKYNNYATYYADAVKD | 9 | AENIGTSYISYWAY |
| G4_KDDD_huCD3_I2E2_29019_EKK_ | | | | | | |
| heteroIgG(1zSEFL2v503YTE)_ | | | | | | |
| HC_E huCD3 HV | | | | | | |
| DLL3_0- | 10 | GSSTGAVTSGNYPN | 11 | GTKFLAP | 12 | VLWYSNRWV |
| G4_KDDD_huCD3_I2E2_29019_EKK_ | | | | | | |
| heteroIgG(1zSEFL2v503YTE)_ | | | | | | |
| LC_K huCD3 LV | | | | | | |

Table 6A. huCD3 CDR Sequences

| Seq | | |
|---|---|---|
| ID | | |
| No. | Name | Sequence |
| 7 | DLL3_8-A7_KDDD_huCD3_29019_EKK_heteroIgG(1zSEFL2v503YTE)_HC_E_huCD3_HV_CDR1 | KYAIN |
| 8 | DLL3_8-A7 | RIRSKYNNYATYYADAVKD |
| | KDDD_huCD3_29019_EKK_heteroIgG(1zSEFL2v503YTE)_HC_E_huCD3_HV_CDR2 | |
| 19 | DLL3_8-A7 | AGNFGSSYISYWAY |
| | KDDD_huCD3_29019_EKK_heteroIgG(1zSEFL2v503YTE)_HC_E_huCD3_HV_CDR3 | |
| 10 | DLL3_8-A7_KDDD_huCD3_29019_EKK_heteroIgG(1zSEFL2v503YTE)_LC_K_huCD3_LV_CDR1 | GSSTGAVTSGNYPN |
| 11 | DLL3_8-A7 | GTKFLAP |
| | KDDD_huCD3_29019_EKK_heteroIgG(1zSEFL2v503YTE)_LC_K_huCD3_LV_CDR2 | |
| 12 | DLL3_8-A7_KDDD_huCD3_29019_EKK_heteroIgG(1zSEFL2v503YTE) | VLWYSNRWV |
| | LC_K_huCD3_LV_CDR3 | |
| 7 | huDLL3(40861)_huCD3(29019(VH: G110E_F112I_S114T))_heteroIgG4PE-FALA-KiH_HC_E_huCD3_HV_CDR1 | KYAIN |
| 8 | huDLL3(40861)_huCD3(29019(VH: G110E_F112I_S114T))_heteroIgG4PE-FALA-KiH_HC_E_huCD3_HV_CDR2 | RIRSKYNNYATYYADAVKD |
| 9 | huDLL3(40861)_huCD3(29019(VH: G110E_F112I_S114T))_heteroIgG4PE-FALA-KiH_HC_E_huCD3_HV_CDR3 | AENIGTSYISYWAY |
| 10 | huDLL3(40861)_huCD3(29019(VH: G110E_F112I_S114T))_heteroIgG4PE-FALA-KiH_LC_K_huCD3_LV_CDR1 | GSSTGAVTSGNYPN |
| 11 | huDLL3(40861)_huCD3(29019(VH: G110E_F112I_S114T))_heteroIgG4PE-FALA-KiH_LC_K_huCD3_LV_CDR2 | GTKFLAP |
| 12 | huDLL3(40861)_huCD3(29019(VH: G110E_F112I_S114T))_heteroIgG4PE-FALA-KiH_LC_K_huCD3_LV | VLWYSNRWV |
| | CDR3 | |
| 7 | DLL3_8-A7_KDDD_huCD3_I2E2_29019_EKK_heteroIgG(1zSEFL2v503YTE)_HC_E_huCD3_HV_CDR1 | KYAIN |
| 8 | DLL3_8-A7 | RIRSKYNNYATYYADAVKD |
| | KDDD_huCD3_I2E2_29019_EKK_heteroIgG(1zSEFL2v503YTE)_HC_E_huCD3_HV_CDR2 | |
| 9 | DLL3_8-A7_KDDD_huCD3_I2E2_29019_EKK_heteroIgG(1zSEFL2v503YTE)_HC_E_huCD3_HV_CDR3 | AENIGTSYISYWAY |
| 10 | DLL3_8-A7_KDDD_huCD3_I2E2_29019_EKK_heteroIgG(1zSEFL2v503YTE)_LC_K_huCD3_LV_CDR1 | GSSTGAVTSGNYPN |
| 11 | DLL3_8-A7 | GTKFLAP |
| | KDDD_huCD3_I2E2_29019_EKK_heteroIgG(1zSEFL2v503YTE)_LC_K_huCD3_LV_CDR2 | |
| 12 | DLL3_8-A7_KDDD_huCD3_I2E2_29019_EKK_heteroIgG(1zSEFL2v503YTE)_LC_K_huCD3_LV_CDR3 | VLWYSNRWV |
| 7 | DLL3_0-G4_KDDD_huCD3_I2E2_29019_EKK_heteroIgG(1zSEFL2v503YTE)_HC_E_huCD3_HV_CDR1 | KYAIN |
| 8 | DLL3_0-G4_KDDD_huCD3_I2E2_29019_EKK_heteroIgG(1zSEFL2v503YTE)_HC_E_huCD3_HV_CDR2 | RIRSKYNNYATYYADAVKD |
| 9 | DLL3_0-G4_KDDD | AENIGTSYISYWAY |
| | huCD3_I2E2_29019_EKK_heteroIgG(1zSEFL2v503YTE)_HC_E_huCD3_HV_CDR3 | |
| 10 | DLL3_0-G4_KDDD_huCD3_I2E2_29019_EKK_heteroIgG(1zSEFL2v503YTE)_LC_K_huCD3_LV_CDR1 | GSSTGAVTSGNYPN |
| 11 | DLL3_0-G4_KDDD_huCD3_I2E2_29019_EKK_heteroIgG(1zSEFL2v503YTE)_LC_K_huCD3_LV_CDR2 | GTKFLAP |
| 12 | DLL3_0-G4_KDDD_huCD3_I2E2_29019_EKK_heteroIgG(1zSEFL2v503YTE)_LC_K_huCD3_LV_CDR3 | VLWYSNRWV |

TABLE 7
huDLL3 CDR Sequences

| | Seq | | Seq | | Seq | |
|---|---|---|---|---|---|---|
| | ID | | ID | | ID | |
| Name | No. | CDR1 | No. | CDR2 | No. | CDR3 |
| DLL3_8-A7 | 13 | SYYWS | 14 | YVYYSGTTNYNPSLKS | 15 | IAVTGFYFDY |
| KDDD_huCD3_29019_EKK_heteroIgG(1zSEFL2v503YTE)_ | | | | | | |
| HC_K_huDLL3 HV | | | | | | |
| DLL3_8-A7 | 16 | RASQRVNNNYLA | 17 | GASSRAT | 18 | QQYDRSPLT |
| KDDD_huCD3_29019_EKK_heteroIgG(1zSEFL2v503YTE)_ | | | | | | |
| LC_E_huDLL3 LV | | | | | | |
| huDLL3(40861)_huCD3(29019(VH: G110E_F112I_ | 1 | NYGMH | 2 | VISHHGSSKYYARSVKG | 3 | DWWELVFDY |
| S114T))_heteroIgG4PE-FALA-KiH_HC_K_huDLL3_HV | | | | | | |
| huDLL3(40861)_huCD3(29019(VH: G110E_F112I_ | 4 | KSSQSLLHSDGKTFLY | 5 | EVSNRFS | 6 | LQGIHLPFT |
| S114T))_heteroIgG4PE-FALA-KiH_LC_E_huDLL3_LV | | | | | | |
| DLL3_8- | 13 | SYYWS | 14 | YVYYSGTTNYNPSLKS | 15 | IAVTGFYFDY |
| A7_KDDD_huCD3_I2E2_29019_EKK_heteroIgG | | | | | | |
| (1zSEFL2v503YTE)_HC_K huDLL3_HV | | | | | | |
| DLL3_8- | 16 | RASQRVNNNYLA | 17 | GASSRAT | 18 | QQYDRSPLT |
| A7_KDDD_huCD3_I2E2_29019_EKK_heteroIgG | | | | | | |
| (1zSEFL2v503YTE)_LC_E huDLL3_LV | | | | | | |
| DLL3_0- | 1 | NYGMH | 2 | VISHHGSSKYYARSVKG | 3 | DWWELVFDY |
| G4_KDDD_huCD3_I2E2_29019_EKK_heteroIgG | | | | | | |
| (1zSEFL2v503YTE)_HC_K huDLL3_HV | | | | | | |
| DLL3_0- | 4 | KSSQSLLHSDGKTFLY | 5 | EVSNRFS | 6 | LQGIHLPFT |
| G4_KDDD_huCD3_I2E2_29019_EKK_heteroIgG | | | | | | |
| (1zSEFL2v503YTE)_LC_E huDLL3_LV | | | | | | |

Table 7A. huDLL3 CDR Sequences

| Seq | | |
|---|---|---|
| ID | | |
| No. | Name | Sequence |
| 13 | DLL3_8-A7_KDDD_huCD3_29019_EKK_heteroIgG(1zSEFL2v503YTE)_HC_K_huDLL3_HV_CDR1 | SYYWS |
| 14 | DLL3_8-A7_KDDD_huCD3_29019_EKK_heteroIgG(1zSEFL2v503YTE)_HC_K_huDLL3_HV_CDR2 | YVYYSGTTNYNPSLKS |
| 15 | DLL3_8-A7_KDDD_huCD3_29019_EKK_heteroIgG(1zSEFL2v503YTE)_HC_K_huDLL3_HV_CDR3 | IAVTGFYFDY |
| 16 | DLL3_8-A7_KDDD_huCD3_29019_EKK_heteroIgG(1zSEFL2v503YTE)_LC_E_huDLL3_LV_CDR1 | RASQRVNNNYLA |
| 17 | DLL3_8-A7_KDDD_huCD3_29019_EKK_heteroIgG(1zSEFL2v503YTE)_LC_E_huDLL3_LV_CDR2 | GASSRAT |
| 18 | DLL3_8-A7_KDDD_huCD3_29019_EKK_heteroIgG(1zSEFL2v503YTE)_LC_E_huDLL3_LV_CDR3 | QQYDRSPLT |
| 1 | huDLL3(40861)_huCD3(29019(VH: G110E_F112I_S114T))_heteroIgG4PE-FALA-KiH_HC_K_huDLL3_HV_CDR1 | NYGMH |
| 2 | huDLL3(40861)_huCD3(29019(VH: G110E_F112I_S114T))_heteroIgG4PE-FALA-KiH_HC_K_huDLL3_HV_CDR2 | VISHHGSSKYYARSVKG |
| 3 | huDLL3(40861)_huCD3(29019(VH: G110E_F112I_S114T))_heteroIgG4PE-FALA-KiH_HC_K_huDLL3_HV_CDR3 | DWWELVFDY |
| 4 | huDLL3(40861)_huCD3(29019(VH: G110E_F112I_S114T))_heteroIgG4PE-FALA-KiH_LC_E_huDLL3_LV_CDR1 | KSSQSLLHSDGKTFLY |
| 5 | huDLL3(40861)_huCD3(29019(VH: G110E_F112I_S114T))_heteroIgG4PE-FALA-KiH_LC_E_huDLL3_LV_CDR2 | EVSNRFS |
| 6 | huDLL3(40861)_huCD3(29019(VH: G110E_F112I_S114T))_heteroIgG4PE-FALA-KiH_LC_E_huDLL3_LV_CDR3 | LQGIHLPFT |
| 13 | DLL3_8-A7_KDDD_huCD3_I2E2_29019_EKK_heteroIgG(1zSEFL2v503YTE)_HC_K_huDLL3_HV_CDR1 | SYYWS |
| 14 | DLL3_8-A7_KDDD_huCD3_I2E2_29019_EKK_heteroIgG(1zSEFL2v503YTE)_HC_K_huDLL3_HV_CDR2 | YVYYSGTTNYNPSLKS |
| 15 | DLL3_8-A7_KDDD_huCD3_I2E2_29019_EKK_heteroIgG(1zSEFL2v503YTE)_HC_K_huDLL3_HV_CDR3 | IAVTGFYFDY |
| 16 | DLL3_8-A7_KDDD_huCD3_I2E2_29019_EKK_heteroIgG(1zSEFL2v503YTE)_LC_E_huDLL3_LV_CDR1 | RASQRVNNNYLA |
| 17 | DLL3_8-A7_KDDD_huCD3_I2E2_29019_EKK_heteroIgG(1zSEFL2v503YTE)_LC_E_huDLL3_LV_CDR2 | GASSRAT |
| 18 | DLL3_8-A7_KDDD_huCD3_I2E2_29019_EKK_heteroIgG(1zSEFL2v503YTE)_LC_E_huDLL3_LV_CDR3 | QQYDRSPLT |
| 1 | DLL3_0-G4_KDDD_huCD3_I2E2_29019_EKK_heteroIgG(1zSEFL2v503YTE)_HC_K_huDLL3_HV_CDR1 | NYGMH |
| 2 | DLL3_0-G4_KDDD_huCD3_I2E2_29019_EKK_heteroIgG(1zSEFL2v503YTE)_HC_K_huDLL3_HV_CDR2 | VISHHGSSKYYARSVKG |
| 3 | DLL3_0-G4_KDDD_huCD3_I2E2_29019_EKK_heteroIgG(1zSEFL2v503YTE)_HC_K_huDLL3_HV_CDR3 | DWWELVFDY |
| 4 | DLL3_0-G4_KDDD_huCD3_I2E2_29019_EKK_heteroIgG(1zSEFL2v503YTE)_LC_E_huDLL3_LV_CDR1 | KSSQSLLHSDGKTFLY |
| 5 | DLL3_0-G4_KDDD_huCD3_I2E2_29019_EKK_heteroIgG(1zSEFL2v503YTE)_LC_E_huDLL3_LV_CDR2 | EVSNRFS |
| 6 | DLL3_0-G4_KDDD_huCD3_I2E2_29019_EKK_heteroIgG(1zSEFL2v503YTE)_LC_E_huDLL3_LV_CDR3 | LQGIHLPFT |

TABLE 8
huCD3 Framework Sequences

| | Seq | | Seq | | Seq | | Seq | |
|---|---|---|---|---|---|---|---|---|
| | ID | | ID | | ID | | ID | |
| Name | No. | FR1 | No. | FR2 | No. | FR3 | No. | FR4 |
| DLL3_8-A7 | 28 | EVQLVESGGGLVQPGGS | 21 | WVRQAPGKGL | 29 | RFTISRDDSKNTVYLQMNNLKT | 23 | WGQGTL |
| KDDD_huCD3_29019_EKK_heteroIgG | | LKLSCAASGFTFN | | EWVA | | EDTAVYYCAR | | VTVSS |
| (1zSEFL2v503YTE)_HC_E huCD3 HV | | | | | | | | |
| DLL3_8-A7 | 30 | QTVVTQEPSLTVSPGGT | 31 | WVQKKPGQAP | 32 | GTPARFSGSLSGGKAALTLSGV | 33 | FGSGTKL |
| KDDD_huCD3_29019_EKK_heteroIgG | | VTITC | | RGLIG | | QPEDEAEYYC | | TVLG |
| (1zSEFL2v503YTE)LC K huCD3 LV | | | | | | | | |
| huDLL3(40861)_huCD3(29019(VH: G110E_ | 28 | EVQLVESGGGLVQPGGS | 21 | WVRQAPGKGL | 29 | RFTISRDDSKNTVYLQMNNLKT | 23 | WGQGTL |
| F112I_S114T)) heteroIgG4PE-FALA- | | LKLSCAASGFTFN | | EWVA | | EDTAVYYCAR | | VTVSS |
| KiH HC E huCD3 HV | | | | | | | | |
| huDLL3(40861)_huCD3(29019(VH: G110E_ | 30 | QTVVTQEPSLTVSPGGT | 31 | WVQKKPGQAP | 32 | GTPARFSGSLSGGKAALTLSGV | 33 | FGSGTKL |
| F112I_S114T))_heteroIgG4PE- | | VTITC | | RGLIG | | QPEDEAEYYC | | TVLG |
| FALA-KiH_LC_K huCD3 LV | | | | | | | | |
| DLL3_8- | 28 | EVQLVESGGGLVQPGGS | 21 | WVRQAPGKGL | 29 | RFTISRDDSKNTVYLQMNNLKT | 23 | WGQGTL |
| A7_KDDD_huCD3_I2E2_29019_EKK_heteroIgG | | LKLSCAASGFTFN | | EWVA | | EDTAVYYCAR | | VTVSS |
| (1zSEFL2v503YTE)_HC_E huCD3 HV | | | | | | | | |
| DLL3_8- | 30 | QTVVTQEPSLTVSPGGT | 31 | WVQKKPGQAP | \|32 | GTPARFSGSLSGGKAALTLSGV | 33 | FGSGTKL |
| A7_KDDD_huCD3_I2E2_29019_EKK_heteroIgG | | VTITC | | RGLIG | | QPEDEAEYYC | | TVLG |
| (1zSEFL2v503YTE) LC K huCD3 LV | | | | | | | | |
| DLL3_0- | 28 | EVQLVESGGGLVQPGGS | 21 | WVRQAPGKGL | 29 | RFTISRDDSKNTVYLQMNNLKT | 23 | WGQGTL |
| G4_KDDD_huCD3_I2E2_29019_EKK_heteroIgG | | LKLSCAASGFTFN | | EWVA | | EDTAVYYCAR | | VTVSS |
| (1zSEFL2v503YTE)_HC_E huCD3 HV | | | | | | | | |
| DLL3_0- | 30 | QTVVTQEPSLTVSPGGT | 31 | WVQKKPGQAP | 32 | GTPARFSGSLSGGKAALTLSGV | 33 | FGSGTKL |
| G4_KDDD_huCD3_I2E2_29019_EKK_heteroIgG | | VTITC | | RGLIG | | QPEDEAEYYC | | TVLG |
| (1zSEFL2v503YTE) LC K huCD3 LV | | | | | | | | |

Table 8A. huCD3 Framework Sequences

| Seq | | |
|---|---|---|
| ID | | |
| No. | Name | Sequence |
| 28 | DLL3_8-A7_KDDD_huCD3_29019_EKK_heteroIgG(1zSEFL2v503YTE)_HC_E huCD3 HV FR1 | EVQLVESGGGLVQPGGSLKLSCAASGFTEN |
| 21 | DLL3_8-A7_KDDD_huCD3_29019_EKK_heteroIgG(1zSEFL2v503YTE)_HC_E huCD3 HV FR2 | WVRQAPGKGLEWVA |
| 29 | DLL3_8-A7_KDDD_huCD3_29019_EKK_heteroIgG(1zSEFL2v503YTE)_HC_E huCD3 HV FR3 | RFTISRDDSKNTVYLQMNNLKTEDTAVYYCAR |
| 23 | DLL3_8-A7_KDDD_huCD3_29019_EKK_heteroIgG(1zSEFL2v503YTE)_HC_E huCD3 HV FR4 | WGQGTLVTVSS |
| 30 | DLL3_8-A7_KDDD_huCD3_29019_EKK_heteroIgG(1zSEFL2v503YTE)_LC_K huCD3 LV FR1 | QTVVTQEPSLTVSPGGTVTITC |
| 31 | DLL3_8-A7_KDDD_huCD3_29019_EKK_heteroIgG(1zSEFL2v503YTE)_LC_K huCD3 LV FR2 | WVQKKPGQAPRGLIG |
| 32 | DLL3_8-A7_KDDD_huCD3_29019_EKK_heteroIgG(1zSEFL2v503YTE)_LC_K huCD3 LV FR3 | GTPARFSGSLSGGKAALTLSGVQPEDEAEYYC |
| 33 | DLL3_8-A7_KDDD_huCD3_29019_EKK_heteroIgG(1zSEFL2v503YTE)_LC_K huCD3 LV FR4 | FGSGTKLTVLG |
| 28 | huDLL3(40861)_huCD3(29019(VH: G110E_F112I_S114T))_heteroIgG4PE-FALA-KiH_HC_E | EVQLVESGGGLVQPGGSLKLSCAASGFTFN |
| | huCD3 HV FR1 | |
| 21 | huDLL3(40861)_huCD3(29019(VH: G110E_F112I_S114T))_heteroIgG4PE-FALA-KiH_HC_E | WVRQAPGKGLEWVA |
| | huCD3 HV FR2 | |
| 29 | huDLL3(40861)_huCD3(29019(VH: G110E_F112I_S114T))_heteroIgG4PE-FALA-KiH_HC_E | RFTISRDDSKNTVYLQMNNLKTEDTAVYYCAR |
| | huCD3 HV FR3 | |
| 23 | huDLL3(40861)_huCD3(29019(VH: G110E_F112I_S114T))_heteroIgG4PE-FALA-KiH_HC_E | WGQGTLVTVSS |
| | huCD3 HV FR4 | |
| 30 | huDLL3(40861)_huCD3(29019(VH: G110E_F112I_S114T))_heteroIgG4PE-FALA-KiH_LC_K | QTVVTQEPSLTVSPGGTVTITC |
| | huCD3 LV FR1 | |
| 31 | huDLL3(40861)_huCD3(29019(VH: G110E_F112I_S114T))_heteroIgG4PE-FALA-KiH_LC_K | WVQKKPGQAPRGLIG |
| | huCD3 LV FR2 | |
| 32 | huDLL3(40861)_huCD3(29019(VH: G110E_F112I_S114T))_heteroIgG4PE-FALA-KiH_LC_K | GTPARFSGSLSGGKAALTLSGVQPEDEAEYYC |
| | huCD3 LV FR3 | |
| 33 | huDLL3(40861)_huCD3(29019(VH: G110E_F112I_S114T))_heteroIgG4PE-FALA-KiH_LC_K | FGSGTKLTVLG |
| | huCD3 LV FR4 | |
| 28 | DLL3_8-A7_KDDD_huCD3_I2E2_29019_EKK_heteroIgG(1zSEFL2v503YTE)_HC_E_huCD3 | EVQLVESGGGLVQPGGSLKLSCAASGFTFN |
| | HV FR1 | |
| 21 | DLL3_8-A7_KDDD_huCD3_I2E2_29019_EKK_heteroIgG(1zSEFL2v503YTE)_HC_E_huCD3 | WVRQAPGKGLEWVA |
| | HV FR2 | |
| 29 | DLL3_8-A7_KDDD_huCD3_I2E2_29019_EKK_heteroIgG(1zSEFL2v503YTE)_HC_E_huCD3 | RFTISRDDSKNTVYLQMNNLKTEDTAVYYCAR |
| | HV FR3 | |
| 23 | DLL3_8-A7_KDDD_huCD3_I2E2_29019_EKK_heteroIgG(1zSEFL2v503YTE)_HC_E_huCD3 | WGQGTLVTVSS |
| | HV FR4 | |
| 30 | DLL3_8-A7_KDDD_huCD3_I2E2_29019_EKK_heteroIgG(1zSEFL2v503YTE)_LC_K_huCD3 | QTVVTQEPSLTVSPGGTVTITC |
| | LV FR1 | |
| 31 | DLL3_8-A7_KDDD_huCD3_I2E2_29019_EKK_heteroIgG(1zSEFL2v503YTE)_LC_K_huCD3 | WVQKKPGQAPRGLIG |
| | LV FR2 | |
| 32 | DLL3_8-A7_KDDD_huCD3_I2E2_29019_EKK_heteroIgG(1zSEFL2v503YTE)_LC_K_huCD3 | GTPARFSGSLSGGKAALTLSGVQPEDEAEYYC |
| | LV FR3 | |
| 33 | DLL3_8-A7_KDDD_huCD3_I2E2_29019_EKK_heteroIgG(1zSEFL2v503YTE)_LC_K_huCD3 | FGSGTKLTVLG |
| | LV FR4 | |
| 28 | DLL3_0-G4_KDDD_huCD3_I2E2_29019_EKK_heteroIgG(1zSEFL2v503YTE)_HC_E_huCD3 | EVQLVESGGGLVQPGGSLKLSCAASGFTFN |
| | HV FR1 | |
| 21 | DLL3_0-G4_KDDD_huCD3_I2E2_29019_EKK_heteroIgG(1zSEFL2v503YTE)_HC_E_huCD3 | WVRQAPGKGLEWVA |
| | HV FR2 | |
| 29 | DLL3_0-G4_KDDD_huCD3_I2E2_29019_EKK_heteroIgG(1zSEFL2v503YTE)_HC_E_huCD3 | RFTISRDDSKNTVYLQMNNLKTEDTAVYYCAR |
| | HV FR3 | |
| 23 | DLL3_0-G4_KDDD_huCD3_I2E2_29019_EKK_heteroIgG(1zSEFL2v503YTE)_HC_E_huCD3 | WGQGTLVTVSS |
| | HV FR4 | |
| 30 | DLL3_0-G4_KDDD_huCD3_I2E2_29019_EKK_heteroIgG(1zSEFL2v503YTE)_LC_K_huCD3 | QTVVTQEPSLTVSPGGTVTITC |
| | LV FR1 | |
| 31 | DLL3_0-G4_KDDD_huCD3_I2E2_29019_EKK_heteroIgG(1zSEFL2v503YTE)_LC_K_huCD3 | WVQKKPGQAPRGLIG |
| | LV FR2 | |
| 32 | DLL3_0-G4_KDDD_huCD3_I2E2_29019_EKK_heteroIgG(1zSEFL2v503YTE)_LC_K_huCD3 | GTPARFSGSLSGGKAALTLSGVQPEDEAEYYC |
| | LV FR3 | |
| 33 | DLL3_0-G4_KDDD_huCD3_I2E2_29019_EKK_heteroIgG(1zSEFL2v503YTE)_LC_K_huCD3 | FGSGTKLTVLG |
| | LV FR4 | |

TABLE 9
huDLL3 Framework Sequences

| | Seq | | Seq | | Seq ID | | Seq ID | |
|---|---|---|---|---|---|---|---|---|
| Name | ID No. | FR1 | ID No. | FR2 | No. | FR3 | No. | FR4 |
| DLL3_8-A7 | 34 | QVQLQESGPGLVKPSET | 35 | WIRQPPGKGLEWIG | 36 | RVTISVDTSKNQFSLK | 23 | WGQGTLVT |
| KDDD_huCD3_29019_EKK_heteroIgG | | LSLTCTVSGGSIS | | | | LSSVTAADTAVYYCA | | VSS |
| (1zSEFL2v503YTE) HC K huDLL3 HV | | | | | | S | | |
| DLL3_8-A7 | 37 | EIVLTQSPGTLSLSPGER | 38 | WYQQRPGQAPRLLIY | 39 | GIPDRFSGSGSGTDFT | 40 | FGGGTKLEI |
| KDDD_huCD3_29019_EKK_heteroIgG | | VTLSC | | | | LTISRLEPEDFAVYYC | | KR |
| (1zSEFL2v503YTE)_LC_E huDLL3 LV | | | | | | | | |
| huDLL3(40861)_huCD3(29019(VH: G110E_ | 20 | QVQLVESGGGAVQPGR | 21 | WVRQAPGKGLEWVA | 22 | RFTISRDNSKNTLYLE | 23 | WGQGTLVT |
| F112I_S114T))_heteroIgG4PE-FALA-KiH_HC_ | | SLRLSCAASGFTFS | | | | MNSLRAEDTAVYYC | | VSS |
| K huDLL3_HV | | | | | | AR | | |
| huDLL3(40861)_huCD3(29019(VH: G110E_ | 24 | DIVMTQTPLSLSVTPGQ | 25 | WYLQKPGQPPQLLIY | 26 | GVPDRFSGSGSGTDFTLKISRV | 27 | FGPGTKVEI |
| F112I_S114T))_heteroIgG4PE-FALA-KiH_LC_ | | PASISC | | | | EAEDVGVYYC | | KR |
| E huDLL3_LV | | | | | | | | |
| DLL3_8- | 34 | QVQLQESGPGLVKPSET | 35 | WIRQPPGKGLEWIG | 36 | RVTISVDTSKNQFSLK | 23 | WGQGTLVT |
| A7_KDDD_huCD3_I2E2_29019_EKK_heteroIgG | | LSLTCTVSGGSIS | | | | LSSVTAADTAVYYCA | | VSS |
| (1zSEFL2v503YTE)_HC_K huDLL3 HV | | | | | | S | | |
| DLL3_8- | 37 | EIVLTQSPGTLSLSPGER | 38 | WYQQRPGQAPRLLIY | 39 | GIPDRFSGSGSGTDFT | 40 | FGGGTKLEI |
| A7_KDDD_huCD3_I2E2_29019_EKK_heteroIgG | | VTLSC | | | | LTISRLEPEDFAVYYC | | KR |
| (1zSEFL2v503YTE)_LC_E huDLL3 LV | | | | | | | | |
| DLL3_0- | 20 | QVQLVESGGGAVQPGR | 21 | WVRQAPGKGLEWVA | 22 | RFTISRDNSKNTLYLEMNSLRA | 23 | WGQGTLVT |
| G4_KDDD_huCD3_I2E2_29019_EKK_heteroIgG | | SLRLSCAASGFTFS | | | | EDTAVYYCAR | | VSS |
| (1zSEFL2v503YTE) HC K huDLL3 HV | | | | | | | | |
| DLL3_0- | 24 | DIVMTQTPLSLSVTPGQ | 25 | WYLQKPGQPPQLLIY | 26 | GVPDRFSGSGSGTDFTLKISRV | 27 | FGPGTKVEI |
| G4_KDDD_huCD3_I2E2_29019_EKK_heteroIgG | | PASISC | | | | EAEDVGVYYC | | KR |
| (1zSEFL2v503YTE)_LC_E huDLL3 LV | | | | | | | | |

Table 9A. huDLL3 Framework Sequences

| Seq | | |
|---|---|---|
| ID | | |
| No. | Name | Sequence |
| 34 | DLL3_8-A7_KDDD_huCD3_29019_EKK_heteroIgG(1zSEFL2v503YTE)_HC_K huDLL3 HV | QVQLQESGPGLVKPSETLSLTCTVSGGSIS |
| | FR1 | |
| 35 | DLL3_8-A7_KDDD_huCD3_29019_EKK_heteroIgG(1zSEFL2v503YTE)_HC_K huDLL3 HV | WIRQPPGKGLEWIG |
| | FR2 | |
| 36 | DLL3_8-A7_KDDD_huCD3_29019_EKK_heteroIgG(1zSEFL2v503YTE)_HC_K huDLL3 HV | RVTISVDTSKNQFSLKLSSVTAADTAVYYCAS |
| | FR3 | |
| 23 | DLL3_8-A7_KDDD_huCD3_29019_EKK_heteroIgG(1zSEFL2v503YTE)_HC_K huDLL3 HV | WGQGTLVTVSS |
| | FR4 | |
| 37 | DLL3_8-A7_KDDD_huCD3_29019_EKK_heteroIgG(1zSEFL2v503YTE)_LC_E_huDLL3_LV_FR1 | EIVLTQSPGTLSLSPGERVTLSC |
| 38 | DLL3_8-A7_KDDD_huCD3_29019_EKK_heteroIgG(1zSEFL2v503YTE)_LC_E_huDLL3_LV_FR2 | WYQQRPGQAPRLLIY |
| 39 | DLL3_8-A7_KDDD_huCD3_29019_EKK_heteroIgG(1zSEFL2v503YTE)_LC_E_huDLL3_LV_FR3 | GIPDRFSGSGSGTDFTLTISRLEPEDFAVYYC |
| 40 | DLL3_8-A7_KDDD_huCD3_29019_EKK_heteroIgG(1zSEFL2v503YTE)_LC_E_huDLL3_LV_FR4 | FGGGTKLEIKR |
| 20 | huDLL3(40861)_huCD3(29019(VH: G110E_F112I_S114T))_heteroIgG4PE-FALA-KiH_HC_K | QVQLVESGGGAVQPGRSLRLSCAASGFTFS |
| | huDLL3 HV FR1 | |
| 21 | huDLL3(40861)_huCD3(29019(VH: G110E_F112I_S114T))_heteroIgG4PE-FALA-KiH_HC_K | WVRQAPGKGLEWVA |
| | huDLL3 HV FR2 | |
| 22 | huDLL3(40861)_huCD3(29019(VH: G110E_F112I_S114T))_heteroIgG4PE-FALA-KiH_HC_K | RFTISRDNSKNTLYLEMNSLRAEDTAVYYCAR |
| | huDLL3 HV FR3 | |
| 23 | huDLL3(40861)_huCD3(29019(VH: G110E_F112I_S114T))_heteroIgG4PE-FALA-KiH_HC_K | WGQGTLVTVSS |
| | huDLL3 HV FR4 | |
| 24 | huDLL3(40861)_huCD3(29019(VH: G110E_F112I_S114T))_heteroIgG4PE-FALA-KiH_LC_E | DIVMTQTPLSLSVTPGQPASISC |
| | huDLL3 LV FR1 | |
| 25 | huDLL3(40861)_huCD3(29019(VH: G110E_F112I_S114T))_heteroIgG4PE-FALA-KiH_LC_E | WYLQKPGQPPQLLIY |
| | huDLL3 LV FR2 | |
| 26 | huDLL3(40861)_huCD3(29019(VH: G110E_F112I_S114T))_heteroIgG4PE-FALA-KiH_LC_E | GVPDRFSGSGSGTDFTLKISRVEAEDVGVYYC |
| | huDLL3 LV FR3 | |
| 27 | huDLL3(40861)_huCD3(29019(VH: G110E_F112I_S114T))_heteroIgG4PE-FALA-KiH_LC_E | FGPGTKVEIKR |
| | huDLL3 LV FR4 | |
| 34 | DLL3_8-A7_KDDD_huCD3_I2E2_29019_EKK_heteroIgG(1zSEFL2v503YTE)_HC_K huDLL3 | QVQLQESGPGLVKPSETLSLTCTVSGGSIS |
| | HV FR1 | |
| 35 | DLL3_8-A7_KDDD_huCD3_I2E2_29019_EKK_heteroIgG(1zSEFL2v503YTE)_HC_K huDLL3 | WIRQPPGKGLEWIG |
| | HV FR2 | |
| 36 | DLL3_8-A7_KDDD_huCD3_I2E2_29019_EKK_heteroIgG(1zSEFL2v503YTE)_HC_K huDLL3 | RVTISVDTSKNQFSLKLSSVTAADTAVYYCAS |
| | HV FR3 | |
| 23 | DLL3_8-A7_KDDD_huCD3_I2E2_29019_EKK_heteroIgG(1zSEFL2v503YTE)_HC_K huDLL3 | WGQGTLVTVSS |
| | HV FR4 | |
| 37 | DLL3_8-A7_KDDD_huCD3_I2E2_29019_EKK_heteroIgG(1zSEFL2v503YTE)_LC_E huDLL3 | EIVLTQSPGTLSLSPGERVTLSC |
| | LV FR1 | |
| 38 | DLL3_8-A7_KDDD_huCD3_I2E2_29019_EKK_heteroIgG(1zSEFL2v503YTE)_LC_E huDLL3 | WYQQRPGQAPRLLIY |
| | LV FR2 | |
| 39 | DLL3_8-A7_KDDD_huCD3_I2E2_29019_EKK_heteroIgG(1zSEFL2v503YTE)_LC_E huDLL3 | GIPDRFSGSGSGTDFTLTISRLEPEDFAVYYC |
| | LV FR3 | |
| 40 | DLL3_8-A7_KDDD_huCD3_I2E2_29019_EKK_heteroIgG(1zSEFL2v503YTE)_LC_E huDLL3 | FGGGTKLEIKR |
| | LV FR4 | |
| 20 | DLL3_0-G4_KDDD_huCD3_I2E2_29019_EKK_heteroIgG(1zSEFL2v503YTE)_HC_K huDLL3 | QVQLVESGGGAVQPGRSLRLSCAASGFTFS |
| | HV FR1 | |
| 21 | DLL3_0-G4_KDDD_huCD3_I2E2_29019_EKK_heteroIgG(1zSEFL2v503YTE)_HC_K huDLL3 | WVRQAPGKGLEWVA |
| | HV FR2 | |
| 22 | DLL3_0-G4_KDDD_huCD3_I2E2_29019_EKK_heteroIgG(1zSEFL2v503YTE)_HC_K huDLL3 | RFTISRDNSKNTLYLEMNSLRAEDTAVYYCAR |
| | HV FR3 | |
| 23 | DLL3_0-G4_KDDD_huCD3_I2E2_29019_EKK_heteroIgG(1zSEFL2v503YTE)_HC_K huDLL3 | WGQGTLVTVSS |
| | HV FR4 | |
| 24 | DLL3_0-G4_KDDD_huCD3_I2E2_29019_EKK_heteroIgG(1zSEFL2v503YTE)_LC_E huDLL3 | DIVMTQTPLSLSVTPGQPASISC |
| | LV FR1 | |
| 25 | DLL3_0-G4_KDDD_huCD3_I2E2_29019_EKK_heteroIgG(1zSEFL2v503YTE)_LC_E huDLL3 | WYLQKPGQPPQLLIY |
| | LV FR2 | |
| 26 | DLL3_0-G4_KDDD_huCD3_I2E2_29019_EKK_heteroIgG(1zSEFL2v503YTE)_LC_E huDLL3 | GVPDRFSGSGSGTDFTLKISRVEAEDVGVYYC |
| | LV FR3 | |
| 27 | DLL3_0-G4_KDDD_huCD3_I2E2_29019_EKK_heteroIgG(1zSEFL2v503YTE)_LC_E huDLL3 | FGPGTKVEIKR |
| | LV FR4 | |

TABLE 10
huDLL3 V Region Sequences

| | Seq | | Seq | |
|---|---|---|---|---|
| | ID | | ID | |
| Name | No. | HC_K HV | No. | LC_E LV |
| DLL3_8-A7 | 45 | QVQLQESGPGLVKPSETLSLTCTVSGGSISSYYWSWIRQ | 46 | EIVLTQSPGTLSLSPGERVTLSCRASQRV |
| KDDD_huCD3_ | | PPGKGLEWIGYVYYSGTTNYNPSLKSRVTISVDTSKNQF | | NNNYLAWYQQRPGQAPRLLIYGASSRATG |
| 29019_EKK_ | | SLKLSSVTAADTAVYYCASIAVTGFYFDYWGQGTLVTVS | | IPDRFSGSGSGTDFTLTISRLEPEDFAVY |
| heteroIgG | | S | | YCQQYDRSPLTFGGGTKLEIKR |
| (1zSEFL2v503YTE) | | | | |
| huDLL3(40861)_ | 41 | QVQLVESGGGAVQPGRSLRLSCAASGFTFSNYGMHWVRQ | 42 | DIVMTQTPLSLSVTPGQPASISCKSSQSL |
| huCD3(29019 | | APGKGLEWVAVISHHGSSKYYARSVKGRFTISRDNSKNT | | LHSDGKTFLYWYLQKPGQPPQLLIYEVSN |
| (VH: G110E_ | | LYLEMNSLRAEDTAVYYCARDWWELVFDYWGQGTLVTVS | | RFSGVPDRFSGSGSGTDFTLKISRVEAEDV |
| F112I_S114T))_ | | S | | GVYYCLQGIHLPFTFGPGTKVEIKR |
| heteroIgG4PE- | | | | |
| FALA-KiH | | | | |
| DLL3_8- | 45 | QVQLQESGPGLVKPSETLSLTCTVSGGSISSYYWSWIRQ | 46 | EIVLTQSPGTLSLSPGERVTLSCRASQRV |
| A7_KDDD_huCD3_ | | PPGKGLEWIGYVYYSGTTNYNPSLKSRVTISVDTSKNQF | | NNNYLAWYQQRPGQAPRLLIYGASSRATG |
| I2E2_29019_ | | SLKLSSVTAADTAVYYCASIAVTGFYFDYWGQGTLVTVS | | IPDRFSGSGSGTDFTLTISRLEPEDFAVY |
| EKK_heteroIgG | | S | | YCQQYDRSPLTFGGGTKLEIKR |
| (1zSEFL2v503YTE) | | | | |
| DLL3_0- | 41 | QVQLVESGGGAVQPGRSLRLSCAASGFTFSNYGMHWVRQ | 42 | DIVMTQTPLSLSVTPGQPASISCKSSQSL |
| G4_KDDD_huCD3_ | | APGKGLEWVAVISHHGSSKYYARSVKGRFTISRDNSKNT | | LHSDGKTFLYWYLQKPGQPPQLLIYEVSN |
| I2E2_29019_EKK_ | | LYLEMNSLRAEDTAVYYCARDWWELVFDYWGQGTLVTVS | | RFSGVPDRFSGSGSGTDFTLKISRVEAED |
| heteroIgG | | S | | VGVYYCLQGIHLPFTFGPGTKVEIKR |
| (1zSEFL2v503YTE) | | | | |

Table 10A. huDLL3 V Region Sequences

| Seq | | |
|---|---|---|
| ID | | |
| No. | Name | Sequence |
| 45 | DLL3_8-A7 | QVQLQESGPGLVKPSETLSLTCTVSGGSISSYYWSWIRQPPGKGLEWIGYVYYS |
| | KDDD_huCD3_29019_EKK_heteroIgG | GTTNYNPSLKSRVTISVDTSKNQFSLKLSSVTAADTAVYYCASIAVTGFYFDYW |
| | (1zSEFL2v503YTE) HC K HV | GQGTLVTVSS |
| 46 | DLL3_8-A7 | EIVLTQSPGTLSLSPGERVTLSCRASQRVNNNYLAWYQQRPGQAPRLLIYGASS |
| | KDDD_huCD3_29019_EKK heteroIgG | RATGIPDRFSGSGSGTDFTLTISRLEPEDFAVYYCQQYDRSPLTFGGGTKLEIK |
| | (1zSEFL2v503YTE) LC ELV | R |
| 41 | huDLL3(40861)_huCD3(29019(VH: | QVQLVESGGGAVQPGRSLRLSCAASGFTFSNYGMHWVRQAPGKGLEWVAVISHH |
| | G110E_F112I S114T))_heteroIgG4PE- | GSSKYYARSVKGRFTISRDNSKNTLYLEMNSLRAEDTAVYYCARDWWELVFDYW |
| | FALA-KiH HC K HV | GQGTLVTVSS |
| 42 | huDLL3(40861)_huCD3(29019(VH: | DIVMTQTPLSLSVTPGQPASISCKSSQSLLHSDGKTFLYWYLQKPGQPPQLLIY |
| | G110E_F112I S114T))_heteroIgG4PE- | EVSNRFSGVPDRFSGSGSGTDFTLKISRVEAEDVGVYYCLQGIHLPFTFGPGTK |
| | FALA-KiH LC E LV | VEIKR |
| 45 | DLL3_8- | QVQLQESGPGLVKPSETLSLTCTVSGGSISSYYWSWIRQPPGKGLEWIGYVYYS |
| | A7_KDDD_huCD3_I2E2_29019_EKK_ | GTTNYNPSLKSRVTISVDTSKNQFSLKLSSVTAADTAVYYCASIAVTGFYFDYW |
| | heteroIgG(1zSEFL2v503YTE) HC_K HV | GQGTLVTVSS |
| 46 | DLL3_8- | EIVLTQSPGTLSLSPGERVTLSCRASQRVNNNYLAWYQQRPGQAPRLLIYGASS |
| | A7_KDDD_huCD3_I2E2_29019_EKK_ | RATGIPDRFSGSGSGTDFTLTISRLEPEDFAVYYCQQYDRSPLTFGGGTKLEIK |
| | heteroIgG(1zSEFL2v503YTE) LC E LV | R |
| 41 | DLL3_0- | QVQLVESGGGAVQPGRSLRLSCAASGFTFSNYGMHWVRQAPGKGLEWVAVISHH |
| | G4_KDDD_huCD3_I2E2_29019_EKK_ | GSSKYYARSVKGRFTISRDNSKNTLYLEMNSLRAEDTAVYYCARDWWELVFDYW |
| | heteroIgG(1zSEFL2v503YTE) HC_K HV | GQGTLVTVSS |
| 42 | DLL3_0- | DIVMTQTPLSLSVTPGQPASISCKSSQSLLHSDGKTFLYWYLQKPGQPPQLLIY |
| | G4_KDDD_huCD3_I2E2_29019_EKK_ | EVSNRFSGVPDRFSGSGSGTDFTLKISRVEAEDVGVYYCLQGIHLPFTFGPGTK |
| | heteroIgG(1zSEFL2v503YTE) LC_E LV | VEIKR |

TABLE 11
huCD3 V Region Sequences

| | Seq | | Seq | |
|---|---|---|---|---|
| | ID | | ID | |
| Name | No. | HC_E HV | No. | LC_KLV |
| DLL3_8-A7 | 47 | EVQLVESGGGLVQPGGSLKLSCAASGFTFNKYAINWVRQAPGKG | 44 | QTVVTQEPSLTVSPGGTVTITCGSSTGAVTSGNYPNWV |
| KDDD_huCD3_29019_ | | LEWVARIRSKYNNYATYYADAVKDRFTISRDDSKNTVYLQMNN | | QKKPGQAPRGLIGGTKFLAPGTPARFSGSLSGGKAALTL |
| EKK_heteroIgG | | LKTEDTAVYYCARAGNFGSSYISYWAYWGQGTLVTVSS | | SGVQPEDEAEYYCVLWYSNRWVFGSGTKLTVLG |
| (1zSEFL2v503YTE) | | | | |
| huDLL3(40861)_huCD3 | 43 | EVQLVESGGGLVQPGGSLKLSCAASGFTFNKYAINWVRQAPGKG | 44 | QTVVTQEPSLTVSPGGTVTITCGSSTGAVTSGNYPNWV |
| (29019(VH: G110E_F112I_ | | LEWVARIRSKYNNYATYYADAVKDRFTISRDDSKNTVYLQMNN | | QKKPGQAPRGLIGGTKFLAPGTPARFSGSLSGGKAALTL |
| S114T))_heteroIgG4PE-FALA- | | LKTEDTAVYYCARAENIGTSYISYWAYWGQGTLVTVSS | | SGVQPEDEAEYYCVLWYSNRWVFGSGTKLTVLG |
| KIH | | | | |
| DLL3_8- | 43 | EVQLVESGGGLVQPGGSLKLSCAASGFTFNKYAINWVRQAPGKG | 44 | QTVVTQEPSLTVSPGGTVTITCGSSTGAVTSGNYPNWV |
| A7_KDDD_huCD3_I2E2_ | | LEWVARIRSKYNNYATYYADAVKDRFTISRDDSKNTVYLQMNN | | QKKPGQAPRGLIGGTKFLAPGTPARFSGSLSGGKAALTL |
| 29019_EKK_heteroIgG | | LKTEDTAVYYCARAENIGTSYISYWAYWGQGTLVTVSS | | SGVQPEDEAEYYCVLWYSNRWVFGSGTKLTVLG |
| (1zSEFL2v503YTE) | | | | |
| DLL3_0- | 43 | EVQLVESGGGLVQPGGSLKLSCAASGFTFNKYAINWVRQAPGKG | 44 | QTVVTQEPSLTVSPGGTVTITCGSSTGAVTSGNYPNWV |
| G4_KDDD_huCD3_I2E2_ | | LEWVARIRSKYNNYATYYADAVKDRFTISRDDSKNTVYLQMNN | | QKKPGQAPRGLIGGTKFLAPGTPARFSGSLSGGKAALTL |
| 29019_EKK_heteroIgG | | LKTEDTAVYYCARAENIGTSYISYWAYWGQGTLVTVSS | | SGVQPEDEAEYYCVLWYSNRWVFGSGTKLTVLG |
| (1zSEFL2v503YTE) | | | | |

Table 11A. huCD3 V Region Sequences

| Seq | | |
|---|---|---|
| ID | | |
| No. | Name | Sequence |
| 47 | DLL3_8-A7 | EVQLVESGGGLVQPGGSLKLSCAASGFTFNKYAINWVRQAPGKGLEWVARIRSKYNNYATYYADAVKDRFTISRD |
| | KDDD_huCD3_29019_EKK_heteroIgG | DSKNTVYLQMNNLKTEDTAVYYCARAGNFGSSYISYWAYWGQGTLVTVSS |
| | (1zSEFL2v503YTE)HC_E HV | |
| 44 | DLL3_8-A7 | QTVVTQEPSLTVSPGGTVTITCGSSTGAVTSGNYPNWVQKKPGQAPRGLIGGTKFLAPGTPARFSGSLSGGKAALTL |
| | KDDD_huCD3_29019_EKK_heteroIgG | SGVQPEDEAEYYCVLWYSNRWVFGSGTKLTVLG |
| | (1zSEFL2v503YTE)LC_KLV | |
| 43 | huDLL3(40861)_huCD3(29019(VH: G110E_ | EVQLVESGGGLVQPGGSLKLSCAASGFTFNKYAINWVRQAPGKGLEWVARIRSKYNNYATYYADAVKDRFTISRD |
| | F112I_S114T))_heteroIgG4PE-FALA- | DSKNTVYLQMNNLKTEDTAVYYCARAENIGTSYISYWAYWGQGTLVTVSS |
| | KIH HC_EHV | |
| 44 | huDLL3(40861)_huCD3(29019(VH: G110E_ | QTVVTQEPSLTVSPGGTVTITCGSSTGAVTSGNYPNWVQKKPGQAPRGLIGGTKFLAPGTPARFSGSLSGGKAALTL |
| | F112I_S114T))_heteroIgG4PE-FALA- | SGVQPEDEAEYYCVLWYSNRWVFGSGTKLTVLG |
| | KiHLC_KLV | |
| 43 | DLL3_8- | EVQLVESGGGLVQPGGSLKLSCAASGFTFNKYAINWVRQAPGKGLEWVARIRSKYNNYATYYADAVKDRFTISRD |
| | A7_KDDD_huCD3_I2E2_29019_EKK_ | DSKNTVYLQMNNLKTEDTAVYYCARAENIGTSYISYWAYWGQGTLVTVSS |
| | heteroIgG(1zSEFL2v503YTE)HC_E HV | |
| 44 | DLL3_8- | QTVVTQEPSLTVSPGGTVTITCGSSTGAVTSGNYPNWVQKKPGQAPRGLIGGTKFLAPGTPARFSGSLSGGKAALTL |
| | A7_KDDD_huCD3_I2E2_29019_EKK_ | SGVQPEDEAEYYCVLWYSNRWVFGSGTKLTVLG |
| | heteroIgG(1zSEFL2v503YTE)LC_KLV | |
| 43 | DLL3 0- | EVQLVESGGGLVQPGGSLKLSCAASGFTFNKYAINWVRQAPGKGLEWVARIRSKYNNYATYYADAVKDRFTISRD |
| | G4_KDDD_huCD3_I2E2_29019_EKK_ | DSKNTVYLQMNNLKTEDTAVYYCARAENIGTSYISYWAYWGQGTLVTVSS |
| | heteroIgG(1zSEFL2v503YTE)HC_E HV | |
| 44 | DLL3_0- | QTVVTQEPSLTVSPGGTVTITCGSSTGAVTSGNYPNWVQKKPGQAPRGLIGGTKFLAPGTPARFSGSLSGGKAALTL |
| | G4_KDDD_huCD3_I2E2_29019_EKK_ | SGVQPEDEAEYYCVLWYSNRWVFGSGTKLTVLG |
| | heteroIgG(1zSEFL2v503YTE)LC_KLV | |

TABLE 12
Constant Region Sequences

| | Seq | | Seq | | Seq | | Seq | LCKLC |
|---|---|---|---|---|---|---|---|---|
| | ID | | ID | | ID | | ID | |
| Name | No. | LC_E LC | No. | HC_E HC | No. | HC_K HC | No. | _ |
| DLL3_8-A7 | 49 | TVAAPSVFIFPPSDEQ | 50 | ASTKGPSVFPLAPSSKSTSGGTAALGCLVKDY | 48 | ASTKGPSVFPLAPSSKSTSGGTAALGCLVK | 51 | QPKAAPSVTLFPPSSE |
| KDDD_huCD3_ | | LKSGTASVVCLLNNF | | FPEPVTVSWNSGALTSGVHTFPAVLQSSGLYS | | DYFPEPVTVSWNSGALTSGVHTFPAVLQSS | | ELQANKATLVCLISDF |
| 29019_EKK_ | | YPREAKVQWKVDNA | | LESVVTVPSSSLGTQTYICNVNHKPSNTKVDK | | GLYSLKSVVTVPSSSLGTQTYICNVNHKPS | | YPGAVTVAWKADSSP |
| heteroIgG | | LQSGNSQESVTEQDS | | KVEPKSCDKTHTCPPCPAPELLGGPSVFLFPP | | NTKVDKKVEPKSCDKTHTCPPCPAPELLGG | | VKAGVETTTPSKQSN |
| (1zSEFL2v503YTE) | | KDSTYSLESTLTLSKA | | KPKDTLYITREPEVTCVVVDVSHEDPEVKFN | | PSVFLFPPKPKDTLYITREPEVTCVVVDVSH | | NKYAAKSYLSLTPEQ |
| | | DYEKHKVYACEVTH | | WYVDGVEVHNAKTKPCEEQYGSTYRCVSVL | | EDPEVKFNWYVDGVEVHNAKTKPCEEQY | | WKSHRSYSCQVTHEG |
| | | QGLSSPVTKSFNRGEC | | TVLHQDWLNGKEYKCKVSNKALPAPIEKTIS | | GSTYRCVSVLTVLHQDWLNGKEYKCKVSN | | STVEKTVAPTECS |
| | | | | KAKGQPREPQVYTLPPSRKEMTKNQVSLTCL | | KALPAPIEKTISKAKGQPREPQVYTLPPSRE | | |
| | | | | VKGFYPSDIAVEWESNGQPENNYKTTPPVLK | | EMTKNQVSLTCLVKGFYPSDIAVEWESNG | | |
| | | | | SDGSFFLYSKLTVDKSRWQQGNVFSCSVMHE | | QPENNYDTTPPVLDSDGSFFLYSDLTVDKS | | |
| | | | | ALHNHYTQKSLSLSPGK | | RWQQGNVFSCSVMHEALHNHYTQDSLSLS | | |
| | | | | | | PGK | | |
| huDLL3(40861)_ | 49 | TVAAPSVFIFPPSDEQ | 53 | ASTKGPSVFPLAPCSRSTSESTAALGCLVKDY | 52 | ASTKGPSVFPLAPCSRSTSESTAALGCLVKD | 51 | QPKAAPSVTLFPPSSE |
| huCD3(29019 | | LKSGTASVVCLLNNF | | FPEPVTVSWNSGALTSGVHTFPAVLQSSGLYS | | YFPEPVTVSWNSGALTSGVHTFPAVLQSSG | | ELQANKATLVCLISDF |
| (VH: G110E_ | | YPREAKVQWKVDNA | | LESVVTVPSSSLGTKTYTCNVDHKPSNTKVD | | LYSLKSVVTVPSSSLGTKTYTCNVDHKPSN | | YPGAVTVAWKADSSP |
| F112I_S114T)) | | LQSGNSQESVTEQDS | | KRVESKYGPPCPPCPAPEAAGGPSVFLFPPKP | | TKVDKRVESKYGPPCPPCPAPEAAGGPSVF | | VKAGVETTTPSKQSN |
| heteroIgG4PE- | | KDSTYSLESTLTLSKA | | KDTLMISRTPEVTCVVVDVSQEDPEVQFNWY | | LFPPKPKDTLMISRTPEVTCVVVDVSQEDPE | | NKYAAKSYLSLTPEQ |
| FALA-KIH | | DYEKHKVYACEVTH | | VDGVEVHNAKTKPREEQFNSTYRVVSVLTVL | | VQFNWYVDGVEVHNAKTKPREEQFNSTYR | | WKSHRSYSCQVTHEG |
| | | QGLSSPVTKSFNRGEC | | HQDWLNGKEYKCKVSNKGLPSSIEKTISKAK | | VVSVLTVLHQDWLNGKEYKCKVSNKGLPS | | STVEKTVAPTECS |
| | | | | GQPREPQVYTLPPSQEEMTKNQVSLSCAVKG | | SIEKTISKAKGQPREPQVYTLPPSQEEMTKN | | |
| | | | | FYPSDIAVEWESNGQPENNYKTTPPVLDSDGS | | QVSLWCLVKGFYPSDIAVEWESNGQPENN | | |
| | | | | FFLVSRLTVDKSRWQEGNVFSCSVMHEALHN | | YKTTPPVLDSDGSFFLYSRLTVDKSRWQEG | | |
| | | | | HYTQKSLSLSLGK | | NVFSCSVMHEALHNHYTQKSLSLSLGK | | |
| DLL3_8- | 49 | TVAAPSVFIFPPSDEQ | 50 | ASTKGPSVFPLAPSSKSTSGGTAALGCLVKDY | 48 | ASTKGPSVFPLAPSSKSTSGGTAALGCLVK | 51 | QPKAAPSVTLFPPSSE |
| A7_KDDD_ | | LKSGTASVVCLLNNF | | FPEPVTVSWNSGALTSGVHTFPAVLQSSGLYS | | DYFPEPVTVSWNSGALTSGVHTFPAVLQSS | | ELQANKATLVCLISDF |
| huCD3_I2E2_ | | YPREAKVQWKVDNA | | LESVVTVPSSSLGTQTYICNVNHKPSNTKVDK | | GLYSLKSVVTVPSSSLGTQTYICNVNHKPS | | YPGAVTVAWKADSSP |
| 29019_EKK_ | | LQSGNSQESVTEQDS | | KVEPKSCDKTHTCPPCPAPELLGGPSVFLFPP | | NTKVDKKVEPKSCDKTHTCPPCPAPELLGG | | VKAGVETTTPSKQSN |
| heteroIgG | | KDSTYSLESTLTLSKA | | KPKDTLYITREPEVTCVVVDVSHEDPEVKFN | | PSVFLFPPKPKDTLYITREPEVTCVVVDVSH | | NKYAAKSYLSLTPEQ |
| (1zSEFL2v503YTE) | | DYEKHKVYACEVTH | | WYVDGVEVHNAKTKPCEEQYGSTYRCVSVL | | EDPEVKFNWYVDGVEVHNAKTKPCEEQY | | WKSHRSYSCQVTHEG |
| | | QGLSSPVTKSFNRGEC | | TVLHQDWLNGKEYKCKVSNKALPAPIEKTIS | | GSTYRCVSVLTVLHQDWLNGKEYKCKVSN | | STVEKTVAPTECS |
| | | | | KAKGQPREPQVYTLPPSRKEMTKNQVSLTCL | | KALPAPIEKTISKAKGQPREPQVYTLPPSRE | | |
| | | | | VKGFYPSDIAVEWESNGQPENNYKTTPPVLK | | EMTKNQVSLTCLVKGFYPSDIAVEWESNG | | |
| | | | | SDGSFFLYSKLTVDKSRWQQGNVFSCSVMHE | | QPENNYDTTPPVLDSDGSFFLYSDLTVDKS | | |
| | | | | ALHNHYTQKSLSLSPGK | | RWQQGNVFSCSVMHEALHNHYTQDSLSLS | | |
| | | | | | | PGK | | |
| DLL3_0- | 49 | TVAAPSVFIFPPSDEQ | 50 | ASTKGPSVFPLAPSSKSTSGGTAALGCLVKDY | 48 | ASTKGPSVFPLAPSSKSTSGGTAALGCLVK | 51 | QPKAAPSVTLFPPSSE |
| G4_KDDD_ | | LKSGTASVVCLLNNF | | FPEPVTVSWNSGALTSGVHTFPAVLQSSGLYS | | DYFPEPVTVSWNSGALTSGVHTFPAVLQSS | | ELQANKATLVCLISDF |
| huCD3_I2E2_ | | YPREAKVQWKVDNA | | LESVVTVPSSSLGTQTYICNVNHKPSNTKVDK | | GLYSLKSVVTVPSSSLGTQTYICNVNHKPS | | YPGAVTVAWKADSSP |
| 29019_EKK_ | | LQSGNSQESVTEQDS | | KVEPKSCDKTHTCPPCPAPELLGGPSVFLFPP | | NTKVDKKVEPKSCDKTHTCPPCPAPELLGG | | VKAGVETTTPSKQSN |
| heteroIgG | | KDSTYSLESTLTLSKA | | KPKDTLYITREPEVTCVVVDVSHEDPEVKFN | | PSVFLFPPKPKDTLYITREPEVTCVVVDVSH | | NKYAAKSYLSLTPEQ |
| (1zSEFL2v503YTE) | | DYEKHKVYACEVTH | | WYVDGVEVHNAKTKPCEEQYGSTYRCVSVL | | EDPEVKFNWYVDGVEVHNAKTKPCEEQY | | WKSHRSYSCQVTHEG |
| | | QGLSSPVTKSFNRGEC | | TVLHQDWLNGKEYKCKVSNKALPAPIEKTIS | | GSTYRCVSVLTVLHQDWLNGKEYKCKVSN | | STVEKTVAPTECS |
| | | | | KAKGQPREPQVYTLPPSRKEMTKNQVSLTCL | | KALPAPIEKTISKAKGQPREPQVYTLPPSRE | | |
| | | | | VKGFYPSDIAVEWESNGQPENNYKTTPPVLK | | EMTKNQVSLTCLVKGFYPSDIAVEWESNG | | |
| | | | | SDGSFFLYSKLTVDKSRWQQGNVFSCSVMHE | | QPENNYDTTPPVLDSDGSFFLYSDLTVDKS | | |
| | | | | ALHNHYTQKSLSLSPGK | | RWQQGNVFSCSVMHEALHNHYTQDSLSLS | | |
| | | | | | | PGK | | |

Table 12A. Constant Region Sequences

| Seq | | |
|---|---|---|
| ID | | |
| No. | Name | Sequence |
| 49 | DLL3_8-A7 | TVAAPSVFIFPPSDEQLKSGTASVVCLLNNFYPREAKVQWKVDNALQSGNSQESVTEQDSKDSTYSLESTLTLSKADYEK |
| | KDDD_huCD3_29019_EKK_heteroIgG | HKVYACEVTHQGLSSPVTKSFNRGEC |
| | (1zSEFL2v503YTE)LC_ELC | |
| 50 | DLL3_8-A7 | ASTKGPSVFPLAPSSKSTSGGTAALGCLVKDYFPEPVTVSWNSGALTSGVHTFPAVLQSSGLYSLESVVTVPSSSLGTQTY |
| | KDDD_huCD3_29019_EKK_ | ICNVNHKPSNTKVDKKVEPKSCDKTHTCPPCPAPELLGGPSVFLFPPKPKDTLYITREPEVTCVVVDVSHEDPEVKFNWY |
| | heteroIgG(1zSEFL2v503YTE) | VDGVEVHNAKTKPCEEQYGSTYRCVSVLTVLHQDWLNGKEYKCKVSNKALPAPIEKTISKAKGQPREPQVYTLPPSRKE |
| | HC_E HC | MTKNQVSLTCLVKGFYPSDIAVEWESNGQPENNYKTTPPVLKSDGSFFLYSKLTVDKSRWQQGNVFSCSVMHEALHNH |
| | | YTQKSLSLSPGK |
| 48 | DLL3_8-A7 | ASTKGPSVFPLAPSSKSTSGGTAALGCLVKDYFPEPVTVSWNSGALTSGVHTFPAVLQSSGLYSLKSVVTVPSSSLGTQTY |
| | KDDD_huCD3_29019_EKK_heteroIgG | ICNVNHKPSNTKVDKKVEPKSCDKTHTCPPCPAPELLGGPSVFLFPPKPKDTLYITREPEVTCVVVDVSHEDPEVKFNWY |
| | (1zSEFL2v503YTE)HC_K HC | VDGVEVHNAKTKPCEEQYGSTYRCVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRWQQGNVFSCSVMHEALHNH |
| | | YTQDSLSLSPGK |
| 51 | DLL3_8-A7 | QPKAAPSVTLFPPSSEELQANKATLVCLISDFYPGAVTVAWKADSSPVKAGVETTTPSKQSNNKYAAKSYLSLTPEQWK |
| | KDDD_huCD3_29019_EKK_heteroIgG | SHRSYSCQVTHEGSTVEKTVAPTECS |
| | (1zSEFL2v503YTE)LC_KLC | |
| 49 | huDLL3(40861)_huCD3(29019(VH: G110E_ | TVAAPSVFIFPPSDEQLKSGTASVVCLLNNFYPREAKVQWKVDNALQSGNSQESVTEQDSKDSTYSLESTLTLSKADYEK |
| | F112I_S114T)) heterolgG4PE-FALA-KiHLC | HKVYACEVTHQGLSSPVTKSFNRGEC |
| | E LC | |
| 53 | huDLL3(40861)_huCD3(29019(VH: | ASTKGPSVFPLAPCSRSTSESTAALGCLVKDYFPEPVTVSWNSGALTSGVHTFPAVLQSSGLYSLESVVTVPSSSLGTKTY |
| | G110E_F112I_S114T))_ | TCNVDHKPSNTKVDKRVESKYGPPCPPCPAPEAAGGPSVFLFPPKPKDTLMISRTPEVTCVVVDVSQEDPEVQFNWYVD |
| | heteroIgG4PE-FALA-KiH HC_E HC | GVEVHNAKTKPREEQFNSTYRVVSVLTVLHQDWLNGKEYKCKVSNKGLPSSIEKTISKAKGQPREPQVYTLPPSQEEMT |
| | | KNQVSLSCAVKGFYPSDIAVEWESNGQPENNYKTTPPVLDSDGSFFLVSRLTVDKSRWQEGNVFSCSVMHEALHNHYT |
| | | QKSLSLSLGK |
| 52 | huDLL3(40861)_huCD3(29019(VH: G110E_ | ASTKGPSVFPLAPCSRSTSESTAALGCLVKDYFPEPVTVSWNSGALTSGVHTFPAVLQSSGLYSLKSVVTVPSSSLGTKTY |
| | F112I_S114T))_ | TCNVDHKPSNTKVDKRVESKYGPPCPPCPAPEAAGGPSVFLFPPKPKDTLMISRTPEVTCVVVDVSQEDPEVQFNWYVD |
| | heteroIgG4PE-FALA-KiH HC_K HC | GVEVHNAKTKPREEQFNSTYRVVSVLTVLHQDWLNGKEYKCKVSNKGLPSSIEKTISKAKGQPREPQVYTLPPSQEEMT |
| | | KNQVSLWCLVKGFYPSDIAVEWESNGQPENNYKTTPPVLDSDGSFFLYSRLTVDKSRWQEGNVFSCSVMHEALHNHYT |
| | | QKSLSLSLGK |
| 51 | huDLL3(40861)_huCD3(29019(VH: G110E_ | QPKAAPSVTLFPPSSEELQANKATLVCLISDFYPGAVTVAWKADSSPVKAGVETTTPSKQSNNKYAAKSYLSLTPEQWK |
| | F112I S114T))_ | SHRSYSCQVTHEGSTVEKTVAPTECS |
| | heteroIgG4PE-FALA-Ki HLC_K LC | |
| 49 | DLL3_8- | TVAAPSVFIFPPSDEQLKSGTASVVCLLNNFYPREAKVQWKVDNALQSGNSQESVTEQDSKDSTYSLESTLTLSKADYEK |
| | A7_KDDD_huCD3_I2E2_29019_EKK heteroIgG | HKVYACEVTHQGLSSPVTKSFNRGEC |
| | (1zSEFL2v503YTE)LC E LC | |
| 50 | DLL3_8- | ASTKGPSVFPLAPSSKSTSGGTAALGCLVKDYFPEPVTVSWNSGALTSGVHTFPAVLQSSGLYSLESVVTVPSSSLGTQTY |
| | A7_KDDD_huCD3_I2E2_29019_EKK_heteroIgG | ICNVNHKPSNTKVDKKVEPKSCDKTHTCPPCPAPELLGGPSVFLFPPKPKDTLYITREPEVTCVVVDVSHEDPEVKFNWY |
| | (1zSEFL2v503YTE)HC E HC | VDGVEVHNAKTKPCEEQYGSTYRCVSVLTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRWQQGNVFSCSVMHEALHNH |
| | | YTQKSLSLSPGK |
| 48 | DLL3_8- | ASTKGPSVFPLAPSSKSTSGGTAALGCLVKDYFPEPVTVSWNSGALTSGVHTFPAVLQSSGLYSLKSVVTVPSSSLGTQTY |
| | A7_KDDD_huCD3_I2E2_29019_EKK_heteroIgG | ICNVNHKPSNTKVDKKVEPKSCDKTHTCPPCPAPELLGGPSVFLFPPKPKDTLYITREPEVTCVVVDVSHEDPEVKFNWY |
| | (1zSEFL2v503YTE)HC_K HC | VDGVEVHNAKTKPCEEQYGSTYRCVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRWQQGNVFSCSVMHEALHNH |
| | | YTQDSLSLSPGK |
| 51 | DLL3_8- | QPKAAPSVTLFPPSSEELQANKATLVCLISDFYPGAVTVAWKADSSPVKAGVETTTPSKQSNNKYAAKSYLSLTPEQWK |
| | A7_KDDD_huCD3_I2E2_29019_EKK_heteroIgG | SHRSYSCQVTHEGSTVEKTVAPTECS |
| | (1zSEFL2v503YTE)LC K LC | |
| 49 | DLL3_0- | TVAAPSVFIFPPSDEQLKSGTASVVCLLNNFYPREAKVQWKVDNALQSGNSQESVTEQDSKDSTYSLESTLTLSKADYEK |
| | G4_KDDD_huCD3_I2E2_29019_EKK_heteroIgG | HKVYACEVTHQGLSSPVTKSFNRGEC |
| | (1zSEFL2v503YTE)LC_E LC | |
| 50 | DLL3_0- | ASTKGPSVFPLAPSSKSTSGGTAALGCLVKDYFPEPVTVSWNSGALTSGVHTFPAVLQSSGLYSLESVVTVPSSSLGTQTY |
| | G4_KDDD_huCD3_I2E2_29019_EKK_heteroIgG | ICNVNHKPSNTKVDKKVEPKSCDKTHTCPPCPAPELLGGPSVFLFPPKPKDTLYITREPEVTCVVVDVSHEDPEVKFNWY |
| | (1zSEFL2v503YTE)HC_E HC | VDGVEVHNAKTKPCEEQYGSTYRCVSVLTVLHQDWLNGKEYKCKVSNKALPAPIEKTISKAKGQPREPQVYTLPPSRKE |
| | | MTKNQVSLTCLVKGFYPSDIAVEWESNGQPENNYKTTPPVLKSDGSFFLYSKLTVDKSRWQQGNVFSCSVMHEALHNH |
| | | YTQKSLSLSPGK |
| 48 | DLL3_0- | ASTKGPSVFPLAPSSKSTSGGTAALGCLVKDYFPEPVTVSWNSGALTSGVHTFPAVLQSSGLYSLKSVVTVPSSSLGTQTY |
| | G4_KDDD_huCD3_I2E2_29019_EKK_heteroIgG | ICNVNHKPSNTKVDKKVEPKSCDKTHTCPPCPAPELLGGPSVFLFPPKPKDTLYITREPEVTCVVVDVSHEDPEVKFNWY |
| | (1zSEFL2v503YTE)HC_K HC | VDGVEVHNAKTKPCEEQYGSTYRCVSVLTVLHQDWLNGKEYKCKVSNKALPAPIEKTISKAKGQPREPQVYTLPPSREE |
| | | MTKNQVSLTCLVKGFYPSDIAVEWESNGQPENNYDTTPPVLDSDGSFFLYSDLTVDKSRWQQGNVFSCSVMHEALHNH |
| | | YTQDSLSLSPGK |
| 51 | DLL3_0- | QPKAAPSVTLFPPSSEELQANKATLVCLISDFYPGAVTVAWKADSSPVKAGVETTTPSKQSNNKYAAKSYLSLTPEQWK |
| | G4_KDDD_huCD3_I2E2_29019_EKK_heteroIgG | SHRSYSCQVTHEGSTVEKTVAPTECS |
| | (1zSEFL2v503YTE)LC_K LC | |

TABLE 13
Full Length-Amino Acid Sequences

| | | Seq | | Seq | | Seq | | Seq | |
|---|---|---|---|---|---|---|---|---|---|
| | | ID | | ID | | ID | | ID | |
| Name | Germline | No. | HC_E | No. | HC_K | No. | LC_E | No. | LC_K |
| DLL3_8- | VH4\|4- | 56 | MDMRVPAQLLGLLLLWLRGARC | 58 | MDMRVPAQLLGLLLLWLR | 59 | MDMRVPAQLLGLLLLWLRG | 57 | MAWALLLLTLLTQGTGSW |
| A7_KDDD_ | 59/D6\|6- | | EVQLVESGGGLVQPGGSLKLSCA | | GARCQVQLQESGPGLVKPS | | ARCEIVLTQSPGTLSLSPGER | | AQTVVTQEPSLTVSPGGTV |
| huCD3_I2E2_ | 19\|RF2/JH4, | | ASGFTFNKYAINWVRQAPGKGL | | ETLSLTCTVSGGSISSYYWS | | VTLSCRASQRVNNNYLAWY | | TITCGSSTGAVTSGNYPNW |
| 29019_EKK_ | VH3\|3- | | EWVARIRSKYNNYATYYADAVK | | WIRQPPGKGLEWIGYVYYS | | QQRPGQAPRLLIYGASSRAT | | VQKKPGQAPRGLIGGTKFL |
| heteroIgG | 73/D2\|2- | | DRFTISRDDSKNTVYLQMNNLKT | | GTTNYNPSLKSRVTISVDTS | | GIPDRFSGSGSGTDFTLTISRL | | APGTPARFSGSLSGGKAAL |
| (1zSEFL2v503YTE) | 21\|RF2/JH4 | | EDTAVYYCARAENIGTSYISYWA | | KNQFSLKLSSVTAADTAVY | | EPEDFAVYYCQQYDRSPLTF | | TLSGVQPEDEAEYYCVLWY |
| | && | | YWGQGTLVTVSSASTKGPSVFPL | | YCASIAVTGFYFDYWGQGT | | GGGTKLEIKRTVAAPSVFIFP | | SNRWVFGSGTKLTVLGQPK |
| | VK3\|A27/JK4, | | APSSKSTSGGTAALGCLVKDYFP | | LVTVSSASTKGPSVFPLAPS | | PSDEQLKSGTASVVCLLNNF | | AAPSVTLFPPSSEELQANKA |
| | VL7\|7b/JL3b | | EPVTVSWNSGALTSGVHTFPAVL | | SKSTSGGTAALGCLVKDYF | | YPREAKVQWKVDNALQSGN | | TLVCLISDFYPGAVTVAWK |
| | | | QSSGLYSLESVVTVPSSSLGTQTY | | PEPVTVSWNSGALTSGVHT | | SQESVTEQDSKDSTYSLESTL | | ADSSPVKAGVETTTPSKQS |
| | | | ICNVNHKPSNTKVDKKVEPKSCD | | FPAVLQSSGLYSLKSVVTVP | | TLSKADYEKHKVYACEVTH | | NNKYAAKSYLSLTPEQWKS |
| | | | KTHTCPPCPAPELLGGPSVFLFPP | | SSSLGTQTYICNVNHKPSNT | | QGLSSPVTKSFNRGEC | | HRSYSCQVTHEGSTVEKTV |
| | | | KPKDTLYITREPEVTCVVVDVSH | | KVDKKVEPKSCDKTHTCPP | | | | |
| | | | EDPEVKFNWYVDGVEVHNAKTK | | CPAPELLGGPSVFLFPPKPK | | | | APTECS |
| | | | PCEEQYGSTYRCVSVLTVLHQD | | DTLYITREPEVTCVVVDVS | | | | |
| | | | WLNGKEYKCKVSNKALPAPIEKT | | HEDPEVKFNWYVDGVEVH | | | | |
| | | | ISKAKGQPREPQVYTLPPSRKEM | | NAKTKPCEEQYGSTYRCVS | | | | |
| | | | TKNQVSLTCLVKGFYPSDIAVEW | | VLTVLHQDWLNGKEYKCK | | | | |
| | | | ESNGQPENNYKTTPPVLKSDGSF | | VSNKALPAPIEKTISKAKGQ | | | | |
| | | | FLYSKLTVDKSRWQQGNVFSCS | | PREPQVYTLPPSREEMTKN | | | | |
| | | | VMHEALHNHYTQKSLSLSPGK | | QVSLTCLVKGFYPSDIAVE | | | | |
| | | | | | WESNGQPENNYDTTPPVLD | | | | |
| | | | | | SDGSFFLYSDLTVDKSRWQ | | | | |
| | | | | | QGNVFSCSVMHEALHNHY | | | | |
| | | | | | TQDSLSLSPGK | | | | |
| huDLL3 | VH3\|3- | 133 | MDMRVPAQLLGLLLLWLRGARC | 60 | MDMRVPAQLLGLLLLWLR | 55 | MDMRVPAQLLGLLLLWLRG | 57 | MAWALLLLTLLTQGTGSW |
| (40861)_ | 30.3/D1\|1- | | EVQLVESGGGLVQPGGSLKLSCA | | GARCQVQLVESGGGAVQP | | ARCDIVMTQTPLSLSVTPGQ | | AQTVVTQEPSLTVSPGGTV |
| huCD3(29019 | 1\|RF3/JH4, | | ASGFTFNKYAINWVRQAPGKGL | | GRSLRLSCAASGFTFSNYG | | PASISCKSSQSLLHSDGKTFL | | TITCGSSTGAVTSGNYPNW |
| (VH: G110E_ | VH3\|3- | | EWVARIRSKYNNYATYYADAVK | | MHWVRQAPGKGLEWVAVI | | YWYLQKPGQPPQLLIYEVSN | | VQKKPGQAPRGLIGGTKFL |
| F112I_S114T))_ | 73/D2\|2- | | DRFTISRDDSKNTVYLQMNNLKT | | SHHGSSKYYARSVKGRFTIS | | RFSGVPDRFSGSGSGTDFTL | | APGTPARFSGSLSGGKAAL |
| heteroIgG4PE- | 21\|RF2/JH4 | | EDTAVYYCARAENIGTSYISYWA | | RDNSKNTLYLEMNSLRAED | | KISRVEAEDVGVYYCLQGIH | | TLSGVQPEDEAEYYCVLWY |
| FALA- | && | | YWGQGTLVTVSSASTKGPSVFPL | | TAVYYCARDWWELVFDY | | LPFTFGPGTKVEIKRTVAAPS | | SNRWVFGSGTKLTVLGQPK |
| KIH | VK2\|A18/JK3, | | APCSRSTSESTAALGCLVKDYFPE | | WGQGTLVTVSSASTKGPSV | | VFIFPPSDEQLKSGTASVVCL | | AAPSVTLFPPSSEELQANKA |
| | VL7\|7b/JL3b | | PVTVSWNSGALTSGVHTFPAVLQ | | FPLAPCSRSTSESTAALGCL | | LNNFYPREAKVQWKVDNAL | | TLVCLISDFYPGAVTVAWK |
| | | | SSGLYSLESVVTVPSSSLGTKTYT | | VKDYFPEPVTVSWNSGALT | | QSGNSQESVTEQDSKDSTYS | | ADSSPVKAGVETTTPSKQS |
| | | | CNVDHKPSNTKVDKRVESKYGP | | SGVHTFPAVLQSSGLYSLKS | | LESTLTLSKADYEKHKVYAC | | NNKYAAKSYLSLTPEQWKS |
| | | | PCPPCPAPEAAGGPSVFLFPPKPK | | VVTVPSSSLGTKTYTCNVD | | EVTHQGLSSPVTKSFNRGEC | | HRSYSCQVTHEGSTVEKTV |
| | | | DTLMISRTPEVTCVVVDVSQEDP | | HKPSNTKVDKRVESKYGPP | | | | APTECS |
| | | | EVQFNWYVDGVEVHNAKTKPRE | | CPPCPAPEAAGGPSVFLFPP | | | | |
| | | | EQFNSTYRVVSVLTVLHQDWLN | | KPKDTLMISRTPEVTCVVV | | | | |
| | | | GKEYKCKVSNKGLPSSIEKTISKA | | DVSQEDPEVQFNWYVDGV | | | | |
| | | | KGQPREPQVYTLPPSQEEMTKNQ | | EVHNAKTKPREEQFNSTYR | | | | |
| | | | VSLSCAVKGFYPSDIAVEWESNG | | VVSVLTVLHQDWLNGKEY | | | | |
| | | | QPENNYKTTPPVLDSDGSFFLVS | | KCKVSNKGLPSSIEKTISKA | | | | |
| | | | RLTVDKSRWQEGNVFSCSVMHE | | KGQPREPQVYTLPPSQEEM | | | | |
| | | | ALHNHYTQKSLSLSLGK | | TKNQVSLWCLVKGFYPSDI | | | | |
| | | | | | AVEWESNGQPENNYKTTPP | | | | |
| | | | | | VLDSDGSFFLYSRLTVDKS | | | | |
| | | | | | RWQEGNVFSCSVMHEALH | | | | |
| | | | | | NHYTQKSLSLSLGK | | | | |
| DLL3_0- | VH3\|3- | 56 | MDMRVPAQLLGLLLLWLRGARC | 54 | MDMRVPAQLLGLLLLWLR | 55 | MDMRVPAQLLGLLLLWLRG | 57 | MAWALLLLTLLTQGTGSW |
| G4_KDDD_ | 30.3/D1\|1- | | EVQLVESGGGLVQPGGSLKLSCA | | GARCQVQLVESGGGAVQP | | ARCDIVMTQTPLSLSVTPGQ | | AQTVVTQEPSLTVSPGGTV |
| huCD3_I2E2_ | 1\|RF3/JH4, | | ASGFTFNKYAINWVRQAPGKGL | | GRSLRLSCAASGFTFSNYG | | PASISCKSSQSLLHSDGKTFL | | TITCGSSTGAVTSGNYPNW |
| 29019_EKK_ | VH3\|3- | | EWVARIRSKYNNYATYYADAVK | | MHWVRQAPGKGLEWVAVI | | YWYLQKPGQPPQLLIYEVSN | | VQKKPGQAPRGLIGGTKFL |
| heteroIgG | 73/D2\|2- | | DRFTISRDDSKNTVYLQMNNLKT | | SHHGSSKYYARSVKGRFTIS | | RFSGVPDRFSGSGSGTDFTL | | APGTPARFSGSLSGGKAAL |
| (1zSEFL2v503YTE) | 21\|RF2/JH4 | | EDTAVYYCARAENIGTSYISYWA | | RDNSKNTLYLEMNSLRAED | | KISRVEAEDVGVYYCLQGIH | | TLSGVQPEDEAEYYCVLWY |
| | && | | YWGQGTLVTVSSASTKGPSVFPL | | TAVYYCARDWWELVFDY | | LPFTFGPGTKVEIKRTVAAPS | | SNRWVFGSGTKLTVLGQPK |
| | VK2\|A18/JK3, | | APSSKSTSGGTAALGCLVKDYFP | | WGQGTLVTVSSASTKGPSV | | VFIFPPSDEQLKSGTASVVCL | | AAPSVTLFPPSSEELQANKA |
| | VL7\|7b/JL3b | | EPVTVSWNSGALTSGVHTFPAVL | | FPLAPSSKSTSGGTAALGCL | | LNNFYPREAKVQWKVDNAL | | TLVCLISDFYPGAVTVAWK |
| | | | QSSGLYSLESVVTVPSSSLGTQTY | | VKDYFPEPVTVSWNSGALT | | QSGNSQESVTEQDSKDSTYS | | ADSSPVKAGVETTTPSKQS |
| | | | ICNVNHKPSNTKVDKKVEPKSCD | | SGVHTFPAVLQSSGLYSLKS | | LESTLTLSKADYEKHKVYAC | | NNKYAAKSYLSLTPEQWKS |
| | | | KTHTCPPCPAPELLGGPSVFLFPP | | VVTVPSSSLGTQTYICNVN | | EVTHQGLSSPVTKSFNRGEC | | HRSYSCQVTHEGSTVEKTV |
| | | | KPKDTLYITREPEVTCVVVDVSH | | HKPSNTKVDKKVEPKSCDK | | | | APTECS |
| | | | EDPEVKFNWYVDGVEVHNAKTK | | THTCPPCPAPELLGGPSVFL | | | | |
| | | | | | FPPKPKDTLYITREPEVTCV | | | | |
| | | | PCEEQYGSTYRCVSVLTVLHQD | | VVDVSHEDPEVKFNWYVD | | | | |
| | | | WLNGKEYKCKVSNKALPAPIEKT | | GVEVHNAKTKPCEEQYGST | | | | |
| | | | ISKAKGQPREPQVYTLPPSRKEM | | YRCVSVLTVLHQDWLNGK | | | | |
| | | | TKNQVSLTCLVKGFYPSDIAVEW | | EYKCKVSNKALPAPIEKTIS | | | | |
| | | | ESNGQPENNYKTTPPVLKSDGSF | | KAKGQPREPQVYTLPPSRE | | | | |
| | | | FLYSKLTVDKSRWQQGNVFSCS | | EMTKNQVSLTCLVKGFYPS | | | | |
| | | | VMHEALHNHYTQKSLSLSPGK | | DIAVEWESNGQPENNYDTT | | | | |
| | | | | | PPVLDSDGSFFLYSDLTVDK | | | | |
| | | | | | SRWQQGNVFSCSVMHEAL | | | | |
| | | | | | HNHYTQDSLSLSPGK | | | | |
| DLL3_8- | VH4\|4- | 132 | MDMRVPAQLLGLLLLWLRGARC | 58 | MDMRVPAQLLGLLLLWLR | 59 | MDMRVPAQLLGLLLLWLRG | 57 | MAWALLLLTLLTQGTGSW |
| A7_KDDD_ | 59/D6\|6 | | EVQLVESGGGLVQPGGSLKLSCA | | GARCQVQLQESGPGLVKPS | | ARCEIVLTQSPGTLSLSPGER | | AQTVVTQEPSLTVSPGGTV |
| huCD3_29019_ | 19\|RF2/JH4, | | ASGFTFNKYAINWVRQAPGKGL | | ETLSLTCTVSGGSISSYYWS | | VTLSCRASQRVNNNYLAWY | | TITCGSSTGAVTSGNYPNW |
| EKK_heteroIgG | VH3\|3- | | EWVARIRSKYNNYATYYADAVK | | WIRQPPGKGLEWIGYVYYS | | QQRPGQAPRLLIYGASSRAT | | VQKKPGQAPRGLIGGTKFL |
| (1zSEFL2v503YTE) | 73/D2\|2- | | DRFTISRDDSKNTVYLQMNNLKT | | GTTNYNPSLKSRVTISVDTS | | GIPDRFSGSGSGTDFTLTISRL | | APGTPARFSGSLSGGKAAL |
| | 21\|RF2/JH4 | | EDTAVYYCARAGNFGSSYISYW | | KNQFSLKLSSVTAADTAVY | | EPEDFAVYYCQQYDRSPLTF | | TLSGVQPEDEAEYYCVLWY |
| | && | | AYWGQGTLVTVSSASTKGPSVFP | | YCASIAVTGFYFDYWGQGT | | GGGTKLEIKRTVAAPSVFIFP | | SNRWVFGSGTKLTVLGQPK |
| | VK3\|A27/JK4, | | LAPSSKSTSGGTAALGCLVKDYF | | LVTVSSASTKGPSVFPLAPS | | PSDEQLKSGTASVVCLLNNF | | AAPSVTLFPPSSEELQANKA |
| | VL7\|7b/JL3b | | PEPVTVSWNSGALTSGVHTFPAV | | SKSTSGGTAALGCLVKDYF | | YPREAKVQWKVDNALQSGN | | TLVCLISDFYPGAVTVAWK |
| | | | LQSSGLYSLESVVTVPSSSLGTQT | | PEPVTVSWNSGALTSGVHT | | SQESVTEQDSKDSTYSLESTL | | ADSSPVKAGVETTTPSKQS |
| | | | YICNVNHKPSNTKVDKKVEPKSC | | FPAVLQSSGLYSLKSVVTVP | | TLSKADYEKHKVYACEVTH | | NNKYAAKSYLSLTPEQWKS |
| | | | DKTHTCPPCPAPELLGGPSVFLFP | | SSSLGTQTYICNVNHKPSNT | | QGLSSPVTKSFNRGEC | | HRSYSCQVTHEGSTVEKTV |
| | | | PKPKDTLYITREPEVTCVVVDVS | | KVDKKVEPKSCDKTHTCPP | | | | APTECS |
| | | | HEDPEVKFNWYVDGVEVHNAKT | | CPAPELLGGPSVFLFPPKPK | | | | |
| | | | KPCEEQYGSTYRCVSVLTVLHQD | | DTLYITREPEVTCVVVDVS | | | | |
| | | | WLNGKEYKCKVSNKALPAPIEKT | | HEDPEVKFNWYVDGVEVH | | | | |
| | | | ISKAKGQPREPQVYTLPPSRKEM | | NAKTKPCEEQYGSTYRCVS | | | | |
| | | | TKNQVSLTCLVKGFYPSDIAVEW | | VLTVLHQDWLNGKEYKCK | | | | |
| | | | ESNGQPENNYKTTPPVLKSDGSF | | VSNKALPAPIEKTISKAKGQ | | | | |
| | | | FLYSKLTVDKSRWQQGNVFSCS | | PREPQVYTLPPSREEMTKN | | | | |
| | | | VMHEALHNHYTQKSLSLSPGK | | QVSLTCLVKGFYPSDIAVE | | | | |
| | | | | | WESNGQPENNYDTTPPVLD | | | | |
| | | | | | SDGSFFLYSDLTVDKSRWQ | | | | |
| | | | | | QGNVFSCSVMHEALHNHY | | | | |
| | | | | | TQDSLSLSPGK | | | | |

Table 13A. Full Length-Amino Acid Sequences

| Seq ID | | |
|---|---|---|
| No. | Name | Sequence |
| 56 | DLL3_8- | MDMRVPAQLLGLLLLWLRGARCEVQLVESGGGLVQPGGSLKLSCAASGFTFNKYAINWVRQAPGKGLEWVARIR |
| | A7_KDDD_huCD3_I2E2_29019_EKK_ | SKYNNYATYYADAVKDRFTISRDDSKNTVYLQMNNLKTEDTAVYYCARAENIGTSYISYWAYWGQGTLVTVSSA |
| | heteroIgG(1zSEFL2v503YTE)_HC_E | STKGPSVFPLAPSSKSTSGGTAALGCLVKDYFPEPVTVSWNSGALTSGVHTFPAVLQSSGLYSLESVVTVPSSSLGTQ |
| | | TYICNVNHKPSNTKVDKKVEPKSCDKTHTCPPCPAPELLGGPSVFLFPPKPKDTLYITREPEVTCVVVDVSHEDPEVK |
| | | FNWYVDGVEVHNAKTKPCEEQYGSTYRCVSVLTVLHQDWLNGKEYKCKVSNKALPAPIEKTISKAKGQPREPQVY |
| | | TLPPSRKEMTKNQVSLTCLVKGFYPSDIAVEWESNGQPENNYKTTPPVLKSDGSFFLYSKLTVDKSRWQQGNVFSC |
| | | SVMHEALHNHYTQKSLSLSPGK |
| 58 | DLL3 8- | MDMRVPAQLLGLLLLWLRGARCQVQLQESGPGLVKPSETLSLTCTVSGGSISSYYWSWIRQPPGKGLEWIGYVYYS |
| | A7_KDDD_huCD3_I2E2_29019_EKK_ | GTTNYNPSLKSRVTISVDTSKNQFSLKLSSVTAADTAVYYCASIAVTGFYFDYWGQGTLVTVSSASTKGPSVFPLAP |
| | heteroIgG(1zSEFL2v503YTE)_HC_K | SSKSTSGGTAALGCLVKDYFPEPVTVSWNSGALTSGVHTFPAVLQSSGLYSLKSVVTVPSSSLGTQTYICNVNHKPS |
| | | NTKVDKKVEPKSCDKTHTCPPCPAPELLGGPSVFLFPPKPKDTLYITREPEVTCVVVDVSHEDPEVKFNWYVDGVE |
| | | VHNAKTKPCEEQYGSTYRCVSVLTVLHQDWLNGKEYKCKVSNKALPAPIEKTISKAKGQPREPQVYTLPPSREEMT |
| | | KNQVSLTCLVKGFYPSDIAVEWESNGQPENNYDTTPPVLDSDGSFFLYSDLTVDKSRWQQGNVFSCSVMHEALHN |
| | | HYTQDSLSLSPGK |
| 59 | DLL3_8- | MDMRVPAQLLGLLLLWLRGARCEIVLTQSPGTLSLSPGERVTLSCRASQRVNNNYLAWYQQRPGQAPRLLIYGASS |
| | A7_KDDD_huCD3_I2E2_29019_EKK_ | RATGIPDRFSGSGSGTDFTLTISRLEPEDFAVYYCQQYDRSPLTFGGGTKLEIKRTVAAPSVFIFPPSDEQLKSGTASV |
| | heteroIgG(1zSEFL2v503YTE)_LC_E | VCLLNNFYPREAKVQWKVDNALQSGNSQESVTEQDSKDSTYSLESTLTLSKADYEKHKVYACEVTHQGLSSPVTK |
| | | SFNRGEC |
| 57 | DLL3_8- | MAWALLLLTLLTQGTGSWAQTVVTQEPSLTVSPGGTVTITCGSSTGAVTSGNYPNWVQKKPGQAPRGLIGGTKFL |
| | A7_KDDD_huCD3_I2E2_29019_EKK_ | APGTPARFSGSLSGGKAALTLSGVQPEDEAEYYCVLWYSNRWVFGSGTKLTVLGQPKAAPSVTLFPPSSEELQANK |
| | heteroIgG(1zSEFL2v503YTE)_LC_K | ATLVCLISDFYPGAVTVAWKADSSPVKAGVETTTPSKQSNNKYAAKSYLSLTPEQWKSHRSYSCQVTHEGSTVEKT |
| | | VAPTECS |
| 133 | huDLL3(40861)_huCD3(29019(VH: | MDMRVPAQLLGLLLLWLRGARCEVQLVESGGGLVQPGGSLKLSCAASGFTFNKYAINWVRQAPGKGLEWVARIR |
| | G110E_F112I_S114T))_heteroIgG4PE- | SKYNNYATYYADAVKDRFTISRDDSKNTVYLQMNNLKTEDTAVYYCARAENIGTSYISYWAYWGQGTLVTVSSA |
| | FALA-KIH_HC_E | STKGPSVFPLAPCSRSTSESTAALGCLVKDYFPEPVTVSWNSGALTSGVHTFPAVLQSSGLYSLESVVTVPSSSLGTK |
| | | TYTCNVDHKPSNTKVDKRVESKYGPPCPPCPAPEAAGGPSVFLFPPKPKDTLMISRTPEVTCVVVDVSQEDPEVQFN |
| | | WYVDGVEVHNAKTKPREEQFNSTYRVVSVLTVLHQDWLNGKEYKCKVSNKGLPSSIEKTISKAKGQPREPQVYTL |
| | | PPSQEEMTKNQVSLSCAVKGFYPSDIAVEWESNGQPENNYKTTPPVLDSDGSFFLVSRLTVDKSRWQEGNVFSCSV |
| | | MHEALHNHYTQKSLSLSLGK |
| 60 | huDLL3(40861)_huCD3(29019(VH: | VFPLAPCSRSTSESTAALGCLVKDYFPEPVTVSWNSGALTSGVHTFPAVLQSSGLYSLKSVVTVPSSSLGTKTYTCN |
| | G110E_F112I_S114T))_heteroIgG4PE- | MDMRVPAQLLGLLLLWLRGARCQVQLVESGGGAVQPGRSLRLSCAASGFTFSNYGMHWVRQAPGKGLEWVAVI |
| | FALA-KIH_HC_K | SHHGSSKYYARSVKGRFTISRDNSKNTLYLEMNSLRAEDTAVYYCARDWWELVFDYWGQGTLVTVSSASTKGPS |
| | | VDHKPSNTKVDKRVESKYGPPCPPCPAPEAAGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTISKAKGQPREPQVYTLPPSQE |
| | | EMTKNQVSLWCLVKGFYPSDIAVEWESNGQPENNYKTTPPVLDSDGSFFLYSRLTVDKSRWQEGNVFSCSVMHEA |
| | | LHNHYTQKSLSLSLGK |
| 55 | huDLL3(40861)_huCD3(29019(VH: | MDMRVPAQLLGLLLLWLRGARCDIVMTQTPLSLSVTPGQPASISCKSSQSLLHSDGKTFLYWYLQKPGQPPQLLIYE |
| | G110E_F112I_S114T))_heteroIgG4PE- | VSNRFSGVPDRFSGSGSGTDFTLKISRVEAEDVGVYYCLQGIHLPFTFGPGTKVEIKRTVAAPSVFIFPPSDEQLKSGT |
| | FALA-KIH_LC_E | ASVVCLLNNFYPREAKVQWKVDNALQSGNSQESVTEQDSKDSTYSLESTLTLSKADYEKHKVYACEVTHQGLSSP |
| | | VTKSFNRGEC |
| 57 | huDLL3(40861)_huCD3(29019(VH: | MAWALLLLTLLTQGTGSWAQTVVTQEPSLTVSPGGTVTITCGSSTGAVTSGNYPNWVQKKPGQAPRGLIGGTKFL |
| | G110E_F112I_S114T))_heteroIgG4PE- | APGTPARFSGSLSGGKAALTLSGVQPEDEAEYYCVLWYSNRWVFGSGTKLTVLGQPKAAPSVTLFPPSSEELQANK |
| | FALA-KIH_LC_K | ATLVCLISDFYPGAVTVAWKADSSPVKAGVETTTPSKQSNNKYAAKSYLSLTPEQWKSHRSYSCQVTHEGSTVEKT |
| | | VAPTECS |
| 56 | DLL3_0- | MDMRVPAQLLGLLLLWLRGARCEVQLVESGGGLVQPGGSLKLSCAASGFTFNKYAINWVRQAPGKGLEWVARIR |
| | G4_KDDD_huCD3_I2E2_29019_EKK_ | SKYNNYATYYADAVKDRFTISRDDSKNTVYLQMNNLKTEDTAVYYCARAENIGTSYISYWAYWGQGTLVTVSSA |
| | heteroIgG(1zSEFL2v503YTE)_HC_E | STKGPSVFPLAPSSKSTSGGTAALGCLVKDYFPEPVTVSWNSGALTSGVHTFPAVLQSSGLYSLESVVTVPSSSLGTQ |
| | | TYICNVNHKPSNTKVDKKVEPKSCDKTHTCPPCPAPELLGGPSVFLFPPKPKDTLYITREPEVTCVVVDVSHEDPEVK |
| | | FNWYVDGVEVHNAKTKPCEEQYGSTYRCVSVLTVLHQDWLNGKEYKCKVSNKALPAPIEKTISKAKGQPREPQVY |
| | | TLPPSRKEMTKNQVSLTCLVKGFYPSDIAVEWESNGQPENNYKTTPPVLKSDGSFFLYSKLTVDKSRWQQGNVFSC |
| | | SVMHEALHNHYTQKSLSLSPGK |
| 54 | DLL3_0- | DGVEVHNAKTKPCEEQYGSTYRCVSVLTVLHQDWLNGKEYKCKVSNKALPAPIEKTISKAKGQPREPQVYTLPPSR |
| | G4_KDDD_huCD3_I2E2_29019_EKK_ | MDMRVPAQLLGLLLLWLRGARCQVQLVESGGGAVQPGRSLRLSCAASGFTFSNYGMHWVRQAPGKGLEWVAVI |
| | heteroIgG(1zSEFL2v503YTE)_HC_K | SHHGSSKYYARSVKGRFTISRDNSKNTLYLEMNSLRAEDTAVYYCARDWWELVFDYWGQGTLVTVSSASTKGPS |
| | | VFPLAPSSKSTSGGTAALGCLVKDYFPEPVTVSWNSGALTSGVHTFPAVLQSSGLYSLKSVVTVPSSSLGTQTYICNV |
| | | NHKPSNTKVDKKVEPKSCDKTHTCPPCPAPELLGGPSVFLFPPKPKDTLYITREPEVTCVVVDVSHEDPEVKFNWYV |
| | | EEMTKNQVSLTCLVKGFYPSDIAVEWESNGQPENNYDTTPPVLDSDGSFFLYSDLTVDKSRWQQGNVFSCSVMHE |
| | | ALHNHYTQDSLSLSPGK |
| 55 | DLL3_0- | MDMRVPAQLLGLLLLWLRGARCDIVMTQTPLSLSVTPGQPASISCKSSQSLLHSDGKTFLYWYLQKPGQPPQLLIYE |
| | G4_KDDD_huCD3_I2E2_29019_EKK_ | VSNRFSGVPDRFSGSGSGTDFTLKISRVEAEDVGVYYCLQGIHLPFTFGPGTKVEIKRTVAAPSVFIFPPSDEQLKSGT |
| | heteroIgG(1zSEFL2v503YTE)_LC_E | ASVVCLLNNFYPREAKVQWKVDNALQSGNSQESVTEQDSKDSTYSLESTLTLSKADYEKHKVYACEVTHQGLSSP |
| | | VTKSFNRGEC |
| 57 | DLL3_0- | MAWALLLLTLLTQGTGSWAQTVVTQEPSLTVSPGGTVTITCGSSTGAVTSGNYPNWVQKKPGQAPRGLIGGTKFL |
| | G4_KDDD_huCD3_I2E2_29019_EKK_ | APGTPARFSGSLSGGKAALTLSGVQPEDEAEYYCVLWYSNRWVFGSGTKLTVLGQPKAAPSVTLFPPSSEELQANK |
| | heteroIgG(1zSEFL2v503YTE)_LC_K | ATLVCLISDFYPGAVTVAWKADSSPVKAGVETTTPSKQSNNKYAAKSYLSLTPEQWKSHRSYSCQVTHEGSTVEKT |
| | | VAPTECS |
| 132 | DLL3_8-A7 | TYICNVNHKPSNTKVDKKVEPKSCDKTHTCPPCPAPELLGGPSVFLFPPKPKDTLYITREPEVTCVVVDVSHEDPEVK |
| | KDDD_huCD3_29019_EKK_heteroIgG | MDMRVPAQLLGLLLLWLRGARCEVQLVESGGGLVQPGGSLKLSCAASGFTFNKYAINWVRQAPGKGLEWVARIR |
| | (1zSEFL2v503YTE)_HC_E | SKYNNYATYYADAVKDRFTISRDDSKNTVYLQMNNLKTEDTAVYYCARAGNFGSSYISYWAYWGQGTLVTVSSA |
| | | STKGPSVFPLAPSSKSTSGGTAALGCLVKDYFPEPVTVSWNSGALTSGVHTFPAVLQSSGLYSLESVVTVPSSSLGTQ |
| | | FNWYVDGVEVHNAKTKPCEEQYGSTYRCVSVLTVLHQDWLNGKEYKCKVSNKALPAPIEKTISKAKGQPREPQVY |
| | | TLPPSRKEMTKNQVSLTCLVKGFYPSDIAVEWESNGQPENNYKTTPPVLKSDGSFFLYSKLTVDKSRWQQGNVFSC |
| | | SVMHEALHNHYTQKSLSLSPGK |
| 58 | DLL3_8-A7 | MDMRVPAQLLGLLLLWLRGARCQVQLQESGPGLVKPSETLSLTCTVSGGSISSYYWSWIRQPPGKGLEWIGYVYYS |
| | KDDD_huCD3_29019_EKK_heteroIg | GTTNYNPSLKSRVTISVDTSKNQFSLKLSSVTAADTAVYYCASIAVTGFYFDYWGQGTLVTVSSASTKGPSVFPLAP |
| | (1zSEFL2v503YTE)_HC_K | SSKSTSGGTAALGCLVKDYFPEPVTVSWNSGALTSGVHTFPAVLQSSGLYSLKSVVTVPSSSLGTQTYICNVNHKPS |
| | | NTKVDKKVEPKSCDKTHTCPPCPAPELLGGPSVFLFPPKPKDTLYITREPEVTCVVVDVSHEDPEVKFNWYVDGVE |
| | | VHNAKTKPCEEQYGSTYRCVSVLTVLHQDWLNGKEYKCKVSNKALPAPIEKTISKAKGQPREPQVYTLPPSREEMT |
| | | KNQVSLTCLVKGFYPSDIAVEWESNGQPENNYDTTPPVLDSDGSFFLYSDLTVDKSRWQQGNVFSCSVMHEALHN |
| | | HYTQDSLSLSPGK |
| 59 | DLL3_8-A7 | MDMRVPAQLLGLLLLWLRGARCEIVLTQSPGTLSLSPGERVTLSCRASQRVNNNYLAWYQQRPGQAPRLLIYGASS |
| | KDDD_huCD3_29019_EKK_heteroIgG | RATGIPDRFSGSGSGTDFTLTISRLEPEDFAVYYCQQYDRSPLTFGGGTKLEIKRTVAAPSVFIFPPSDEQLKSGTASV |
| | (1zSEFL2v503YTE)_LC_E | VCLLNNFYPREAKVQWKVDNALQSGNSQESVTEQDSKDSTYSLESTLTLSKADYEKHKVYACEVTHQGLSSPVTK |
| | | SFNRGEC |
| 57 | DLL3_8-A7 | MAWALLLLTLLTQGTGSWAQTVVTQEPSLTVSPGGTVTITCGSSTGAVTSGNYPNWVQKKPGQAPRGLIGGTKFL |
| | KDDD_huCD3_29019_EKK_heteroIgG | APGTPARFSGSLSGGKAALTLSGVQPEDEAEYYCVLWYSNRWVFGSGTKLTVLGQPKAAPSVTLFPPSSEELQANK |
| | (1zSEFL2v503YTE)_LC_K | ATLVCLISDFYPGAVTVAWKADSSPVKAGVETTTPSKQSNNKYAAKSYLSLTPEQWKSHRSYSCQVTHEGSTVEKT |
| | | VAPTECS |

TABLE 14
Full Length-Nucleic Acid Sequences

| | | Seq | | Seq | | Seq | | Seq | |
|---|---|---|---|---|---|---|---|---|---|
| | | ID | | ID | | ID | | ID | |
| Name | Germline | No. | HC_E | No. | HC_K | No. | LC_E | No. | LC_K |
| DLL3_8- | VH4\|4- | 64 | ATGGACATGAGGGTGCCCG | 66 | ATGGACATGAGGGTGCCCG | 67 | ATGGACATGAGGGTGCC | 65 | ATGGCCTGGGCTCTGCTGC |
| A7_KDDD_ | 59/D6\|6- | | CTCAGCTCCTGGGGCTCCT | | CTCAGCTCCTGGGGCTCCT | | CGCTCAGCTCCTGGGGCT | | TCCTCACCCTCCTCACTCA |
| huCD3_I2E2_ | 19\|RF2/JH4, | | GCTGCTGTGGCTGAGAGGT | | GCTGCTGTGGCTGAGAGGT | | CCTGCTGCTGTGGCTGAG | | GGGCACAGGGTCCTGGGC |
| 29019_EKK_ | VH3\|3- | | GCGCGCTGTGAAGTGCAGC | | GCGCGCTGTCAAGTCCAAC | | AGGTGCGCGCTGTGAAAT | | CCAAACAGTGGTCACCCA |
| heteroIgG | 73/D2\|2- | | TGGTTGAATCTGGCGGCGG | | TTCAAGAATCAGGCCCCGG | | TGTACTCACGCAATCACC | | AGAGCCTAGCCTGACCGT |
| (1zSEFL2v503YTE) | 21\|RF2/JH4 | | ATTGGTTCAGCCTGGCGGA | | GCTTGTCAAACCATCTGAA | | TGGTACGCTTTCACTCTC | | TTCTCCTGGCGGCACAGTG |
| | && | | TCTCTGAAGCTGTCTTGTGC | | ACGCTTTCACTCACTTGTAC | | CCCTGGTGAACGAGTCAC | | ACCATCACCTGTGGATCTT |
| | VK3\|A27/ | | CGCCTCTGGCTTCACCTTCA | | GGTCAGTGGTGGGTCCATT | | TCTTAGTTGTCGGGCTTC | | CTACCGGCGCTGTGACCTC |
| | JK4, | | ACAAATACGCCATCAACTG | | TCTTCATATTATTGGAGTTG | | ACAACGCGTCAATAATA | | CGGCAACTACCCTAATTG |
| | VL7\|7b/JL3b | | GGTCCGACAGGCCCCTGGC | | GATTCGGCAACCACCAGGA | | ATTATCTTGCTTGGTATC | | GGTGCAGAAGAAGCCCGG |
| | | | AAAGGACTGGAATGGGTCG | | AAAGGTTTGGAATGGATTG | | AACAACGACCAGGACAA | | CCAGGCTCCTAGAGGACT |
| | | | CCCGGATCAGATCCAAGTA | | GTTATGTCTATTATTCTGGC | | GCTCCACGCTTGCTCATT | | GATCGGAGGCACCAAGTT |
| | | | CAACAACTACGCTACCTAC | | ACTACGAATTATAATCCGT | | TATGGTGCAAGTAGTAGA | | TCTGGCTCCCGGCACTCCT |
| | | | TACGCCGACGCCGTGAAGG | | CACTTAAATCCCGCGTCAC | | GCAACTGGAATTCCTGAT | | GCCAGATTCTCTGGATCTC |
| | | | ACCGGTTCACCATCTCCAG | | TATTTCCGTCGATACTTCAA | | CGGTTTTCCGGGTCTGGG | | TGTCCGGCGGAAAGGCCG |
| | | | AGATGACTCCAAGAACACC | | AGAATCAATTTAGTCTCAA | | AGTGGAACTGATTTTACA | | CTCTGACATTGTCTGGTGT |
| | | | GTCTACCTGCAGATGAACA | | ACTTTCCTCAGTCACGGCT | | TTGACGATTTCTCGCCTC | | CCAGCCTGAGGACGAGGC |
| | | | ACCTGAAAACCGAGGACAC | | GCTGATACTGCTGTTTATTA | | GAACCAGAAGATTTTGCA | | TGAGTACTATTGCGTGCTG |
| | | | CGCCGTGTACTACTGTGCG | | TTGTGCTAGTATTGCAGTCA | | GTTTATTATTGTCAACAA | | TGGTACTCCAACAGATGG |
| | | | CGGGCCGAGAACATCGGAA | | CTGGGTTTTATTTTGATTAT | | TATGATAGATCCCCTCTC | | GTGTTCGGCTCCGGCACC |
| | | | CCTCCTACATCTCCTACTGG | | TGGGGACAAGGTACTCTCG | | ACTTTTGGCGGTGGAACT | | AAGCTGACAGTTCTCGGA |
| | | | GCCTACTGGGGCCAGGGCA | | TCACAGTCTCTTCTGCCTCC | | AAACTTGAAATCAAACG | | CAGCCCAAGGCTGCACCC |
| | | | CACTGGTCACAGTTAGTTC | | ACCAAGGGCCCATCGGTCT | | CACGGTGGCTGCACCATC | | TCGGTCACTCTGTTCCCGC |
| | | | AGCCTCCACCAAGGGCCCA | | TCCCCCTGGCACCCTCCTCC | | TGTCTTCATCTTCCCGCC | | CCTCCTCTGAGGAGCTTCA |
| | | | TCGGTCTTCCCCCTGGCACC | | AAGAGCACCTCTGGGGGCA | | ATCTGATGAGCAGTTGAA | | AGCCAACAAGGCCACACT |
| | | | CTCCTCCAAGAGCACCTCT | | CAGCGGCCCTGGGCTGCCT | | ATCTGGAACTGCCTCTGT | | GGTGTGTCTCATCAGTGAC |
| | | | GGGGGCACAGCGGCCCTGG | | GGTCAAGGACTACTTCCCC | | TGTGTGCCTGCTGAATAA | | TTCTACCCGGGAGCCGTG |
| | | | GCTGCCTGGTCAAGGACTA | | GAACCGGTGACGGTGTCGT | | CTTCTATCCCAGAGAGGC | | ACAGTGGCCTGGAAGGCA |
| | | | CTTCCCCGAACCGGTGACG | | GGAACTCAGGCGCCCTGAC | | CAAAGTACAGTGGAAGG | | GATAGCAGCCCCGTCAAG |
| | | | GTGTCGTGGAACTCAGGCG | | CAGCGGCGTGCACACCTTC | | TGGATAACGCCCTCCAAT | | GCGGGAGTGGAAACCACC |
| | | | CCCTGACCAGCGGCGTGCA | | CCGGCTGTCCTACAGTCCT | | CGGGTAACTCCCAGGAG | | ACACCCTCCAAACAAAGC |
| | | | CACCTTCCCGGCTGTCCTAC | | CAGGACTCTACTCCCTCAA | | AGTGTCACAGAGCAGGA | | AACAACAAGTACGCGGCC |
| | | | AGTCCTCAGGACTCTACTC | | GAGCGTGGTGACCGTGCCC | | CAGCAAGGACAGCACCT | | AAGAGCTATCTGAGCCTG |
| | | | CCTCGAGAGCGTGGTGACC | | TCCAGCAGCTTGGGCACCC | | ACAGCCTCGAAAGCACC | | ACGCCTGAGCAGTGGAAG |
| | | | GTGCCCTCCAGCAGCTTGG | | AGACCTACATCTGCAACGT | | CTGACGCTGAGCAAAGC | | TCCCACAGAAGCTACAGC |
| | | | GCACCCAGACCTACATCTG | | GAATCACAAGCCCAGCAAC | | AGACTACGAGAAACACA | | TGCCAGGTCACGCATGAA |
| | | | CAACGTGAATCACAAGCCC | | ACCAAGGTGGACAAGAAA | | AAGTCTACGCCTGCGAA | | GGGAGCACCGTGGAGAAG |
| | | | AGCAACACCAAGGTGGACA | | GTTGAGCCCAAATCTTGTG | | GTCACCCATCAGGGCCTG | | ACAGTGGCCCCTACAGAA |
| | | | AGAAAGTTGAGCCCAAATC | | ACAAAACTCACACATGCCC | | AGCTCGCCCGTCACAAA | | TGTTCATAG |
| | | | TTGTGACAAAACTCACACA | | ACCGTGCCCAGCACCTGAA | | GAGCTTCAACAGGGGAG | | |
| | | | TGCCCACCGTGCCCAGCAC | | CTCCTGGGGGGACCGTCAG | | AGTGTTAG | | |
| | | | CTGAACTCCTGGGGGGACC | | TCTTCCTCTTCCCCCCAAAA | | | | |
| | | | GTCAGTCTTCCTCTTCCCCC | | CCCAAGGACACCCTCTACA | | | | |
| | | | CAAAACCCAAGGACACCCT | | TCACCCGGGAACCTGAGGT | | | | |
| | | | CTACATCACCCGGGAACCT | | CACATGCGTGGTGGTGGAC | | | | |
| | | | GAGGTCACATGCGTGGTGG | | GTGAGCCACGAAGACCCTG | | | | |
| | | | TGGACGTGAGCCACGAAGA | | AGGTCAAGTTCAACTGGTA | | | | |
| | | | CCCTGAGGTCAAGTTCAAC | | CGTGGACGGCGTGGAGGTG | | | | |
| | | | TGGTACGTGGACGGCGTGG | | CATAATGCCAAGACAAAGC | | | | |
| | | | AGGTGCATAATGCCAAGAC | | CGTGCGAGGAGCAGTACGG | | | | |
| | | | AAAGCCGTGCGAGGAGCA | | CAGCACGTACCGTTGCGTC | | | | |
| | | | GTACGGCAGCACGTACCGT | | AGCGTCCTCACCGTCCTGC | | | | |
| | | | TGCGTCAGCGTCCTCACCG | | ACCAGGACTGGCTGAATGG | | | | |
| | | | TCCTGCACCAGGACTGGCT | | CAAGGAGTACAAGTGCAAG | | | | |
| | | | GAATGGCAAGGAGTACAAG | | GTGTCCAACAAAGCCCTCC | | | | |
| | | | TGCAAGGTGTCCAACAAAG | | CAGCCCCCATCGAGAAAAC | | | | |
| | | | CCCTCCCAGCCCCCATCGA | | CATCTCCAAAGCCAAAGGG | | | | |
| | | | GAAAACCATCTCCAAAGCC | | CAGCCCCGAGAACCACAGG | | | | |
| | | | AAAGGGCAGCCCCGAGAA | | TGTACACCCTGCCCCCATC | | | | |
| | | | CCACAGGTGTACACCCTGC | | CCGGGAGGAGATGACCAA | | | | |
| | | | CCCCATCCCGGAAGGAGAT | | GAACCAGGTCAGCCTGACC | | | | |
| | | | GACCAAGAACCAGGTCAGC | | TGCCTGGTCAAAGGCTTCT | | | | |
| | | | CTGACCTGCCTGGTCAAAG | | ATCCCAGCGACATCGCCGT | | | | |
| | | | GCTTCTATCCCAGCGACAT | | GGAGTGGGAGAGCAATGG | | | | |
| | | | CGCCGTGGAGTGGGAGAGC | | GCAGCCGGAGAACAACTAC | | | | |
| | | | AATGGGCAGCCGGAGAAC | | GACACCACGCCTCCCGTGC | | | | |
| | | | AACTACAAGACCACGCCTC | | TGGACTCCGACGGCTCCTT | | | | |
| | | | CCGTGCTGAAGTCCGACGG | | CTTCCTCTATAGCGACCTCA | | | | |
| | | | CTCCTTCTTCCTCTATAGCA | | CCGTGGACAAGAGCAGGTG | | | | |
| | | | AGCTCACCGTGGACAAGAG | | GCAGCAGGGGAACGTCTTC | | | | |
| | | | CAGGTGGCAGCAGGGGAA | | TCATGCTCCGTGATGCATG | | | | |
| | | | CGTCTTCTCATGCTCCGTGA | | AGGCTCTGCACAACCACTA | | | | |
| | | | TGCATGAGGCTCTGCACAA | | CACGCAGGACAGCCTCTCC | | | | |
| | | | CCACTACACGCAGAAGAGC | | CTGTCTCCGGGCAAATAG | | | | |
| | | | CTCTCCCTGTCTCCGGGCA | | | | | | |
| | | | AATAG | | | | | | |
| huDLL3(40861)_ | VH3\|3- | 135 | ATGGACATGAGGGTGCCCG | 68 | ATGGACATGAGGGTGCCCG | 63 | ATGGACATGAGGGTGCC | 65 | ATGGCCTGGGCTCTGCTGC |
| huCD3 | 30.3/D1\|1- | | CTCAGCTCCTGGGGCTCCT | | CTCAGCTCCTGGGGCTCCT | | CGCTCAGCTCCTGGGGCT | | TCCTCACCCTCCTCACTCA |
| (29019(VH: | 1\|RF3/JH4, | | GCTGCTGTGGCTGAGAGGT | | GCTGCTGTGGCTGAGAGGT | | CCTGCTGCTGTGGCTGAG | | GGGCACAGGGTCCTGGGC |
| G110E_F112I_ | VH3\|3- | | GCGCGCTGTGAAGTGCAGC | | GCGCGCTGTCAAGTTCAAC | | AGGTGCGCGCTGTGATAT | | CCAAACAGTGGTCACCCA |
| S114T))_ | 73/D2\|2- | | TGGTTGAATCTGGCGGCGG | | TTGTCGAATCAGGTGGTGG | | TGTTATGACACAAACACC | | AGAGCCTAGCCTGACCGT |
| heteroIgG4PE- | 21\|RF2/JH4 | | ATTGGTTCAGCCTGGCGGA | | GGCAGTTCAACCAGGGCGC | | TCTTTCACTTTCTGTAAC | | TTCTCCTGGCGGCACAGTG |
| FALA-KiH | && | | TCTCTGAAGCTGTCTTGTGC | | TCATTGCGGTTGTCATGTGC | | ACCGGGACAACCTGCTTC | | ACCATCACCTGTGGATCTT |
| | VK2\|A18/ | | CGCCTCTGGCTTCACCTTCA | | AGCATCAGGTTTTACTTTTA | | CATTTCTTGTAAATCCAG | | CTACCGGCGCTGTGACCTC |
| | JK3, | | ACAAATACGCCATCAACTG | | GTAATTATGGGATGCATTG | | TCAAAGTCTCTTGCATAG | | CGGCAACTACCCTAATTG |
| | VL7\|7b/JL3b | | GGTCCGACAGGCCCCTGGC | | GGTTCGCCAAGCACCTGGA | | TGATGGGAAAACGTTTCT | | GGTGCAGAAGAAGCCCGG |
| | | | AAAGGACTGGAATGGGTCG | | AAAGGCCTCGAATGGGTCG | | CTATTGGTATTTGCAAAA | | CCAGGCTCCTAGAGGACT |
| | | | CCCGGATCAGATCCAAGTA | | CAGTAATTAGTCATCATGG | | GCCTGGACAACCACCGC | | GATCGGAGGCACCAAGTT |
| | | | CAACAACTACGCTACCTAC | | AAGTTCCAAATATTATGCA | | AACTTTTGATTTATGAAG | | TCTGGCTCCCGGCACTCCT |
| | | | TACGCCGACGCCGTGAAGG | | AGAAGCGTCAAAGGTCGCT | | TCAGTAATCGCTTTAGTG | | GCCAGATTCTCTGGATCTC |
| | | | ACCGGTTCACCATCTCCAG | | TTACTATTTCACGCGATAAT | | GAGTTCCAGATCGCTTTT | | TGTCCGGCGGAAAGGCCG |
| | | | AGATGACTCCAAGAACACC | | AGTAAGAACACACTGTATC | | CAGGTTCTGGGTCAGGAA | | CTCTGACATTGTCTGGTGT |
| | | | GTCTACCTGCAGATGAACA | | TTGAGATGAATTCACTCAG | | CAGATTTTACGCTCAAAA | | CCAGCCTGAGGACGAGGC |
| | | | ACCTGAAAACCGAGGACAC | | AGCAGAAGATACTGCTGTC | | TCTCCCGGGTTGAAGCTG | | TGAGTACTATTGCGTGCTG |
| | | | CGCCGTGTACTACTGTGCG | | TATTATTGTGCTAGAGATTG | | AAGATGTTGGGGTCTATT | | TGGTACTCCAACAGATGG |
| | | | CGGGCCGAGAACATCGGAA | | GTGGGAACTTGTATTTGATT | | ATTGTCTTCAAGGGATTC | | GTGTTCGGCTCCGGCACC |
| | | | CCTCCTACATCTCCTACTGG | | ATTGGGGACAAGGAACGCT | | ATCTTCCGTTTACTTTTGG | | AAGCTGACAGTTCTCGGA |
| | | | GCCTACTGGGGCCAGGGCA | | CGTCACAGTATCTTCTGCCT | | GCCAGGGACTAAAGTCG | | CAGCCCAAGGCTGCACCC |
| | | | CACTGGTCACAGTTAGTTC | | CCACCAAGGGGCCATCCGT | | AAATTAAACGGACGGTG | | TCGGTCACTCTGTTCCCGC |
| | | | AGCCTCCACCAAGGGGCCA | | CTTCCCCCTGGCGCCCTGCT | | GCTGCACCATCTGTCTTC | | CCTCCTCTGAGGAGCTTCA |
| | | | TCCGTCTTCCCCCTGGCGCC | | CCAGGAGCACCTCCGAGAG | | ATCTTCCCGCCATCTGAT | | AGCCAACAAGGCCACACT |
| | | | CTGCTCCAGGAGCACCTCC | | CACAGCCGCCCTGGGCTGC | | GAGCAGTTGAAATCTGG | | GGTGTGTCTCATCAGTGAC |
| | | | GAGAGCACAGCCGCCCTGG | | CTGGTCAAGGACTACTTCC | | AACTGCCTCTGTTGTGTG | | TTCTACCCGGGAGCCGTG |
| | | | GCTGCCTGGTCAAGGACTA | | CCGAACCGGTGACGGTGTC | | CCTGCTGAATAACTTCTA | | ACAGTGGCCTGGAAGGCA |
| | | | CTTCCCCGAACCGGTGACG | | GTGGAACTCAGGCGCCCTG | | TCCCAGAGAGGCCAAAG | | GATAGCAGCCCCGTCAAG |
| | | | GTGTCGTGGAACTCAGGCG | | ACCAGCGGCGTGCACACCT | | TACAGTGGAAGGTGGAT | | GCGGGAGTGGAAACCACC |
| | | | CCCTGACCAGCGGCGTGCA | | TCCCGGCTGTCCTACAGTC | | AACGCCCTCCAATCGGGT | | ACACCCTCCAAACAAAGC |
| | | | CACCTTCCCGGCTGTCCTAC | | CTCAGGACTCTACTCCCTC | | AACTCCCAGGAGAGTGT | | AACAACAAGTACGCGGCC |
| | | | AGTCCTCAGGACTCTACTC | | AAGAGCGTGGTGACCGTGC | | CACAGAGCAGGACAGCA | | AAGAGCTATCTGAGCCTG |
| | | | CCTCGAAAGCGTGGTGACC | | CCTCCAGCAGCTTGGGCAC | | AGGACAGCACCTACAGC | | ACGCCTGAGCAGTGGAAG |
| | | | GTGCCCTCCAGCAGCTTGG | | GAAGACCTACACCTGCAAC | | CTCGAAAGCACCCTGAC | | TCCCACAGAAGCTACAGC |
| | | | GCACGAAGACCTACACCTG | | GTAGATCACAAGCCCAGCA | | GCTGAGCAAAGCAGACT | | TGCCAGGTCACGCATGAA |
| | | | CAACGTAGATCACAAGCCC | | ACACCAAGGTGGACAAGA | | ACGAGAAACACAAAGTC | | GGGAGCACCGTGGAGAAG |
| | | | AGCAACACCAAGGTGGACA | | GAGTTGAGTCCAAATATGG | | TACGCCTGCGAAGTCACC | | ACAGTGGCCCCTACAGAA |
| | | | AGAGAGTTGAGTCCAAATA | | TCCCCCATGCCCACCATGC | | CATCAGGGCCTGAGCTCG | | TGTTCATAG |
| | | | TGGTCCCCCATGCCCACCA | | CCAGCACCTGAGGCCGCCG | | CCCGTCACAAAGAGCTTC | | |
| | | | TGCCCAGCACCTGAGGCCG | | GGGGACCATCAGTCTTCCT | | AACAGGGGAGAGTGTTA | | |
| | | | CCGGGGGACCATCAGTCTT | | GTTCCCCCCAAAACCCAAG | | G | | |
| | | | CCTGTTCCCCCCAAAACCC | | GACACTCTCATGATCTCCC | | | | |
| | | | AAGGACACTCTCATGATCT | | GGACCCCTGAGGTCACGTG | | | | |
| | | | CCCGGACCCCTGAGGTCAC | | CGTGGTGGTGGACGTGAGC | | | | |
| | | | GTGCGTGGTGGTGGACGTG | | CAGGAAGACCCCGAGGTCC | | | | |
| | | | AGCCAGGAAGACCCCGAG | | AGTTCAACTGGTACGTGGA | | | | |
| | | | GTCCAGTTCAACTGGTACG | | TGGCGTGGAGGTGCATAAT | | | | |
| | | | TGGATGGCGTGGAGGTGCA | | GCCAAGACAAAGCCGCGG | | | | |
| | | | TAATGCCAAGACAAAGCCG | | GAGGAGCAGTTCAACAGCA | | | | |
| | | | CGGGAGGAGCAGTTCAACA | | CGTACCGTGTGGTCAGCGT | | | | |
| | | | GCACGTACCGTGTGGTCAG | | CCTCACCGTCCTGCACCAG | | | | |
| | | | CGTCCTCACCGTCCTGCAC | | GACTGGCTGAACGGCAAGG | | | | |
| | | | CAGGACTGGCTGAACGGCA | | AGTACAAGTGCAAGGTGTC | | | | |
| | | | AGGAGTACAAGTGCAAGGT | | CAACAAAGGCCTCCCGTCC | | | | |
| | | | GTCCAACAAAGGCCTCCCG | | TCCATCGAGAAAACCATCT | | | | |
| | | | TCCTCCATCGAGAAAACCA | | CCAAAGCCAAAGGGCAGCC | | | | |
| | | | TCTCCAAAGCCAAAGGGCA | | CCGAGAGCCACAGGTGTAC | | | | |
| | | | GCCCCGAGAGCCACAGGTG | | ACCCTGCCCCCATCCCAGG | | | | |
| | | | TACACCCTGCCCCCATCCC | | AGGAGATGACCAAGAACC | | | | |
| | | | AGGAGGAGATGACCAAGA | | AGGTCAGCCTGTGGTGCCT | | | | |
| | | | ACCAGGTCAGCCTGAGCTG | | GGTCAAAGGCTTCTACCCC | | | | |
| | | | CGCCGTCAAAGGCTTCTAC | | AGCGACATCGCCGTGGAGT | | | | |
| | | | CCCAGCGACATCGCCGTGG | | GGGAGAGCAATGGGCAGC | | | | |
| | | | AGTGGGAGAGCAATGGGCA | | CGGAGAACAACTACAAGAC | | | | |
| | | | GCCGGAGAACAACTACAAG | | CACGCCTCCCGTGCTGGAC | | | | |
| | | | ACCACGCCTCCCGTGCTGG | | TCCGACGGCTCCTTCTTCCT | | | | |
| | | | ACTCCGACGGCTCCTTCTTC | | CTACAGCAGGCTAACCGTG | | | | |
| | | | CTCGTGAGCAGGCTAACCG | | GACAAGAGCAGGTGGCAG | | | | |
| | | | TGGACAAGAGCAGGTGGCA | | GAGGGGAATGTCTTCTCAT | | | | |
| | | | GGAGGGGAATGTCTTCTCA | | GCTCCGTGATGCATGAGGC | | | | |
| | | | TGCTCCGTGATGCATGAGG | | TCTGCACAACCACTACACA | | | | |
| | | | CTCTGCACAACCACTACAC | | CAGAAGAGCCTCTCCCTGT | | | | |
| | | | ACAGAAGAGCCTCTCCCTG | | CTCTGGGCAAATAG | | | | |
| | | | TCTCTGGGCAAATAG | | | | | | |
| DLL3_0- | VH3\|3- | 64 | ATGGACATGAGGGTGCCCG | 62 | ATGGACATGAGGGTGCCCG | 63 | ATGGACATGAGGGTGCC | 65 | ATGGCCTGGGCTCTGCTGC |
| G4_KDDD_ | 30.3/D1\|1- | | CTCAGCTCCTGGGGCTCCT | | CTCAGCTCCTGGGGCTCCT | | CGCTCAGCTCCTGGGGCT | | TCCTCACCCTCCTCACTCA |
| huCD3_I2E2_ | 1\|RF3/JH4, | | GCTGCTGTGGCTGAGAGGT | | GCTGCTGTGGCTGAGAGGT | | CCTGCTGCTGTGGCTGAG | | GGGCACAGGGTCCTGGGC |
| 29019_EKK_ | VH3\|3- | | GCGCGCTGTGAAGTGCAGC | | GCGCGCTGTCAAGTTCAAC | | AGGTGCGCGCTGTGATAT | | CCAAACAGTGGTCACCCA |
| heteroIgG | 73/D2\|2- | | TGGTTGAATCTGGCGGCGG | | TTGTCGAATCAGGTGGTGG | | TGTTATGACACAAACACC | | AGAGCCTAGCCTGACCGT |
| (1zSEFL2v503YTE) | 21\|RF2/JH4 | | ATTGGTTCAGCCTGGCGGA | | GGCAGTTCAACCAGGGCGC | | TCTTTCACTTTCTGTAAC | | TTCTCCTGGCGGCACAGTG |
| | && | | TCTCTGAAGCTGTCTTGTGC | | TCATTGCGGTTGTCATGTGC | | ACCGGGACAACCTGCTTC | | ACCATCACCTGTGGATCTT |
| | VK2\|A18/ | | CGCCTCTGGCTTCACCTTCA | | AGCATCAGGTTTTACTTTTA | | CATTTCTTGTAAATCCAG | | CTACCGGCGCTGTGACCTC |
| | JK3, | | ACAAATACGCCATCAACTG | | GTAATTATGGGATGCATTG | | TCAAAGTCTCTTGCATAG | | CGGCAACTACCCTAATTG |
| | VL7\|7b/JL3b | | GGTCCGACAGGCCCCTGGC | | GGTTCGCCAAGCACCTGGA | | TGATGGGAAAACGTTTCT | | GGTGCAGAAGAAGCCCGG |
| | | | AAAGGACTGGAATGGGTCG | | AAAGGCCTCGAATGGGTCG | | CTATTGGTATTTGCAAAA | | CCAGGCTCCTAGAGGACT |
| | | | CCCGGATCAGATCCAAGTA | | CAGTAATTAGTCATCATGG | | GCCTGGACAACCACCGC | | GATCGGAGGCACCAAGTT |
| | | | CAACAACTACGCTACCTAC | | AAGTTCCAAATATTATGCA | | AACTTTTGATTTATGAAG | | TCTGGCTCCCGGCACTCCT |
| | | | TACGCCGACGCCGTGAAGG | | AGAAGCGTCAAAGGTCGCT | | TCAGTAATCGCTTTAGTG | | GCCAGATTCTCTGGATCTC |
| | | | ACCGGTTCACCATCTCCAG | | TTACTATTTCACGCGATAAT | | GAGTTCCAGATCGCTTTT | | TGTCCGGCGGAAAGGCCG |
| | | | AGATGACTCCAAGAACACC | | AGTAAGAACACACTGTATC | | CAGGTTCTGGGTCAGGAA | | CTCTGACATTGTCTGGTGT |
| | | | GTCTACCTGCAGATGAACA | | TTGAGATGAATTCACTCAG | | CAGATTTTACGCTCAAAA | | CCAGCCTGAGGACGAGGC |
| | | | ACCTGAAAACCGAGGACAC | | AGCAGAAGATACTGCTGTC | | TCTCCCGGGTTGAAGCTG | | TGAGTACTATTGCGTGCTG |
| | | | CGCCGTGTACTACTGTGCG | | TATTATTGTGCTAGAGATTG | | AAGATGTTGGGGTCTATT | | TGGTACTCCAACAGATGG |
| | | | CGGGCCGAGAACATCGGAA | | GTGGGAACTTGTATTTGATT | | ATTGTCTTCAAGGGATTC | | GTGTTCGGCTCCGGCACC |
| | | | CCTCCTACATCTCCTACTGG | | ATTGGGGACAAGGAACGCT | | ATCTTCCGTTTACTTTTGG | | AAGCTGACAGTTCTCGGA |
| | | | GCCTACTGGGGCCAGGGCA | | CGTCACAGTATCTTCTGCCT | | GCCAGGGACTAAAGTCG | | CAGCCCAAGGCTGCACCC |
| | | | CACTGGTCACAGTTAGTTC | | CCACCAAGGGCCCATCGGT | | AAATTAAACGGACGGTG | | TCGGTCACTCTGTTCCCGC |
| | | | AGCCTCCACCAAGGGCCCA | | CTTCCCCCTGGCACCCTCCT | | GCTGCACCATCTGTCTTC | | CCTCCTCTGAGGAGCTTCA |
| | | | TCGGTCTTCCCCCTGGCACC | | CCAAGAGCACCTCTGGGGG | | ATCTTCCCGCCATCTGAT | | AGCCAACAAGGCCACACT |
| | | | CTCCTCCAAGAGCACCTCT | | CACAGCGGCCCTGGGCTGC | | GAGCAGTTGAAATCTGG | | GGTGTGTCTCATCAGTGAC |
| | | | GGGGGCACAGCGGCCCTGG | | CTGGTCAAGGACTACTTCC | | AACTGCCTCTGTTGTGTG | | TTCTACCCGGGAGCCGTG |
| | | | GCTGCCTGGTCAAGGACTA | | CCGAACCGGTGACGGTGTC | | CCTGCTGAATAACTTCTA | | ACAGTGGCCTGGAAGGCA |
| | | | CTTCCCCGAACCGGTGACG | | GTGGAACTCAGGCGCCCTG | | TCCCAGAGAGGCCAAAG | | GATAGCAGCCCCGTCAAG |
| | | | GTGTCGTGGAACTCAGGCG | | ACCAGCGGCGTGCACACCT | | TACAGTGGAAGGTGGAT | | GCGGGAGTGGAAACCACC |
| | | | CCCTGACCAGCGGCGTGCA | | TCCCGGCTGTCCTACAGTC | | AACGCCCTCCAATCGGGT | | ACACCCTCCAAACAAAGC |
| | | | CACCTTCCCGGCTGTCCTAC | | CTCAGGACTCTACTCCCTC | | AACTCCCAGGAGAGTGT | | AACAACAAGTACGCGGCC |
| | | | AGTCCTCAGGACTCTACTC | | AAGAGCGTGGTGACCGTGC | | CACAGAGCAGGACAGCA | | AAGAGCTATCTGAGCCTG |
| | | | CCTCGAGAGCGTGGTGACC | | CCTCCAGCAGCTTGGGCAC | | AGGACAGCACCTACAGC | | ACGCCTGAGCAGTGGAAG |
| | | | GTGCCCTCCAGCAGCTTGG | | CCAGACCTACATCTGCAAC | | CTCGAAAGCACCCTGAC | | TCCCACAGAAGCTACAGC |
| | | | GCACCCAGACCTACATCTG | | GTGAATCACAAGCCCAGCA | | GCTGAGCAAAGCAGACT | | TGCCAGGTCACGCATGAA |
| | | | CAACGTGAATCACAAGCCC | | ACACCAAGGTGGACAAGA | | ACGAGAAACACAAAGTC | | GGGAGCACCGTGGAGAAG |
| | | | AGCAACACCAAGGTGGACA | | AAGTTGAGCCCAAATCTTG | | TACGCCTGCGAAGTCACC | | ACAGTGGCCCCTACAGAA |
| | | | AGAAAGTTGAGCCCAAATC | | TGACAAAACTCACACATGC | | CATCAGGGCCTGAGCTCG | | TGTTCATAG |
| | | | TTGTGACAAAACTCACACA | | CCACCGTGCCCAGCACCTG | | CCCGTCACAAAGAGCTTC | | |
| | | | TGCCCACCGTGCCCAGCAC | | AACTCCTGGGGGGACCGTC | | AACAGGGGAGAGTGTTA | | |
| | | | CTGAACTCCTGGGGGGACC | | AGTCTTCCTCTTCCCCCCAA | | G | | |
| | | | GTCAGTCTTCCTCTTCCCCC | | AACCCAAGGACACCCTCTA | | | | |
| | | | CAAAACCCAAGGACACCCT | | CATCACCCGGGAACCTGAG | | | | |
| | | | CTACATCACCCGGGAACCT | | GTCACATGCGTGGTGGTGG | | | | |
| | | | GAGGTCACATGCGTGGTGG | | ACGTGAGCCACGAAGACCC | | | | |
| | | | TGGACGTGAGCCACGAAGA | | TGAGGTCAAGTTCAACTGG | | | | |
| | | | CCCTGAGGTCAAGTTCAAC | | TACGTGGACGGCGTGGAGG | | | | |
| | | | TGGTACGTGGACGGCGTGG | | TGCATAATGCCAAGACAAA | | | | |
| | | | AGGTGCATAATGCCAAGAC | | GCCGTGCGAGGAGCAGTAC | | | | |
| | | | AAAGCCGTGCGAGGAGCA | | GGCAGCACGTACCGTTGCG | | | | |
| | | | GTACGGCAGCACGTACCGT | | TCAGCGTCCTCACCGTCCT | | | | |
| | | | TGCGTCAGCGTCCTCACCG | | GCACCAGGACTGGCTGAAT | | | | |
| | | | TCCTGCACCAGGACTGGCT | | GGCAAGGAGTACAAGTGCA | | | | |
| | | | GAATGGCAAGGAGTACAAG | | AGGTGTCCAACAAAGCCCT | | | | |
| | | | TGCAAGGTGTCCAACAAAG | | CCCAGCCCCCATCGAGAAA | | | | |
| | | | CCCTCCCAGCCCCCATCGA | | ACCATCTCCAAAGCCAAAG | | | | |
| | | | GAAAACCATCTCCAAAGCC | | GGCAGCCCCGAGAACCACA | | | | |
| | | | AAAGGGCAGCCCCGAGAA | | GGTGTACACCCTGCCCCCA | | | | |
| | | | CCACAGGTGTACACCCTGC | | TCCCGGGAGGAGATGACCA | | | | |
| | | | CCCCATCCCGGAAGGAGAT | | AGAACCAGGTCAGCCTGAC | | | | |
| | | | GACCAAGAACCAGGTCAGC | | CTGCCTGGTCAAAGGCTTC | | | | |
| | | | CTGACCTGCCTGGTCAAAG | | TATCCCAGCGACATCGCCG | | | | |
| | | | GCTTCTATCCCAGCGACAT | | TGGAGTGGGAGAGCAATGG | | | | |
| | | | CGCCGTGGAGTGGGAGAGC | | GCAGCCGGAGAACAACTAC | | | | |
| | | | AATGGGCAGCCGGAGAAC | | GACACCACGCCTCCCGTGC | | | | |
| | | | AACTACAAGACCACGCCTC | | TGGACTCCGACGGCTCCTT | | | | |
| | | | CCGTGCTGAAGTCCGACGG | | CTTCCTCTATAGCGACCTCA | | | | |
| | | | CTCCTTCTTCCTCTATAGCA | | CCGTGGACAAGAGCAGGTG | | | | |
| | | | AGCTCACCGTGGACAAGAG | | GCAGCAGGGGAACGTCTTC | | | | |
| | | | CAGGTGGCAGCAGGGGAA | | TCATGCTCCGTGATGCATG | | | | |
| | | | CGTCTTCTCATGCTCCGTGA | | AGGCTCTGCACAACCACTA | | | | |
| | | | TGCATGAGGCTCTGCACAA | | CACGCAGGACAGCCTCTCC | | | | |
| | | | CCACTACACGCAGAAGAGC | | CTGTCTCCGGGCAAATAG | | | | |
| | | | CTCTCCCTGTCTCCGGGCA | | | | | | |
| | | | AATAG | | | | | | |
| DLL3_8-A7 | VH4\|4- | 134 | ATGGACATGAGGGTGCCCG | 66 | ATGGACATGAGGGTGCCCG | 67 | ATGGACATGAGGGTGCC | 70 | ATGGCCTGGGCTCTGCTGC |
| KDDD_huCD3_ | 59/D6\|6- | | CTCAGCTCCTGGGGCTCCT | | CTCAGCTCCTGGGGCTCCT | | CGCTCAGCTCCTGGGGCT | | TCCTCACCCTCCTCACTCA |
| 29019_EKK_ | 19\|RF2/JH4, | | GCTGCTGTGGCTGAGAGGT | | GCTGCTGTGGCTGAGAGGT | | CCTGCTGCTGTGGCTGAG | | GGGCACAGGGTCCTGGGC |
| heteroIgG | VH3\|3- | | GCGCGCTGTGAAGTGCAGC | | GCGCGCTGTCAAGTCCAAC | | AGGTGCGCGCTGTGAAAT | | CCAAACAGTGGTCACCCA |
| (1zSEFL2v503YTE) | 73/D2\|2- | | TGGTTGAATCTGGCGGCGG | | TTCAAGAATCAGGCCCCGG | | TGTACTCACGCAATCACC | | AGAGCCTAGCCTGACCGT |
| | 21\|RF2/JH4 | | ATTGGTTCAGCCTGGCGGA | | GCTTGTCAAACCATCTGAA | | TGGTACGCTTTCACTCTC | | TTCTCCTGGCGGCACCGTG |
| | && | | TCTCTGAAGCTGTCTTGTGC | | ACGCTTTCACTCACTTGTAC | | CCCTGGTGAACGAGTCAC | | ACCATCACCTGTGGATCTT |
| | VK3\|A27/ | | CGCCTCTGGCTTCACCTTCA | | GGTCAGTGGTGGGTCCATT | | TCTTAGTTGTCGGGCTTC | | CTACCGGCGCTGTGACCTC |
| | JK4, | | ACAAATACGCCATCAACTG | | TCTTCATATTATTGGAGTTG | | ACAACGCGTCAATAATA | | CGGCAACTACCCTAATTG |
| | VL7\|7b/JL3b | | GGTCCGACAGGCCCCTGGC | | GATTCGGCAACCACCAGGA | | ATTATCTTGCTTGGTATC | | GGTGCAGAAGAAGCCCGG |
| | | | AAAGGACTGGAATGGGTCG | | AAAGGTTTGGAATGGATTG | | AACAACGACCAGGACAA | | CCAGGCTCCTAGAGGACT |
| | | | CCCGGATCAGATCCAAGTA | | GTTATGTCTATTATTCTGGC | | GCTCCACGCTTGCTCATT | | GATCGGAGGCACCAAGTT |
| | | | CAACAACTACGCTACCTAC | | ACTACGAATTATAATCCGT | | TATGGTGCAAGTAGTAGA | | TCTGGCTCCCGGCACTCCT |
| | | | TACGCCGACGCCGTGAAGG | | CACTTAAATCCCGCGTCAC | | GCAACTGGAATTCCTGAT | | GCCAGATTCTCCGGTTCTC |
| | | | ACCGGTTCACCATCTCCAG | | TATTTCCGTCGATACTTCAA | | CGGTTTTCCGGGTCTGGG | | TGTCTGGCGGAAAGGCCG |
| | | | AGATGACTCCAAGAACACC | | AGAATCAATTTAGTCTCAA | | AGTGGAACTGATTTTACA | | CTCTGACATTGTCTGGCGT |
| | | | GTGTACCTGCAGATGAACA | | ACTTTCCTCAGTCACGGCT | | TTGACGATTTCTCGCCTC | | GCAGCCTGAGGATGAGGC |
| | | | ACCTCAAGACCGAGGACAC | | GCTGATACTGCTGTTTATTA | | GAACCAGAAGATTTTGCA | | TGAGTACTATTGCGTGCTG |
| | | | CGCCGTGTACTACTGTGCC | | TTGTGCTAGTATTGCAGTCA | | GTTTATTATTGTCAACAA | | TGGTACTCCAACAGATGG |
| | | | AGAGCCGGCAACTTCGGCT | | CTGGGTTTTATTTTGATTAT | | TATGATAGATCCCCTCTC | | GTGTTCGGCTCCGGCACC |
| | | | CCTCCTACATCAGCTACTG | | TGGGGACAAGGTACTCTCG | | ACTTTTGGCGGTGGAACT | | AAGCTGACAGTTCTCGGG |
| | | | GGCCTATTGGGGCCAGGGC | | TCACAGTCTCTTCTGCCTCC | | AAACTTGAAATCAAACG | | CAGCCCAAGGCTGCACCC |
| | | | ACACTGGTCACAGTTAGTT | | ACCAAGGGCCCATCGGTCT | | CACGGTGGCTGCACCATC | | TCGGTCACTCTGTTCCCGC |
| | | | CAGCCTCCACCAAGGGCCC | | TCCCCCTGGCACCCTCCTCC | | TGTCTTCATCTTCCCGCC | | CCTCCTCTGAGGAGCTTCA |
| | | | ATCGGTCTTCCCCCTGGCA | | AAGAGCACCTCTGGGGGCA | | ATCTGATGAGCAGTTGAA | | AGCCAACAAGGCCACACT |
| | | | CCCTCCTCCAAGAGCACCT | | CAGCGGCCCTGGGCTGCCT | | ATCTGGAACTGCCTCTGT | | GGTGTGTCTCATCAGTGAC |
| | | | CTGGGGGCACAGCGGCCCT | | GGTCAAGGACTACTTCCCC | | TGTGTGCCTGCTGAATAA | | TTCTACCCGGGAGCCGTG |
| | | | GGGCTGCCTGGTCAAGGAC | | GAACCGGTGACGGTGTCGT | | CTTCTATCCCAGAGAGGC | | ACAGTGGCCTGGAAGGCA |
| | | | TACTTCCCCGAACCGGTGA | | GGAACTCAGGCGCCCTGAC | | CAAAGTACAGTGGAAGG | | GATAGCAGCCCCGTCAAG |
| | | | CGGTGTCGTGGAACTCAGG | | CAGCGGCGTGCACACCTTC | | TGGATAACGCCCTCCAAT | | GCGGGAGTGGAAACCACC |
| | | | CGCCCTGACCAGCGGCGTG | | CCGGCTGTCCTACAGTCCT | | CGGGTAACTCCCAGGAG | | ACACCCTCCAAACAAAGC |
| | | | CACACCTTCCCGGCTGTCCT | | CAGGACTCTACTCCCTCAA | | AGTGTCACAGAGCAGGA | | AACAACAAGTACGCGGCC |
| | | | ACAGTCCTCAGGACTCTAC | | GAGCGTGGTGACCGTGCCC | | CAGCAAGGACAGCACCT | | AAGAGCTATCTGAGCCTG |
| | | | TCCCTCGAGAGCGTGGTGA | | TCCAGCAGCTTGGGCACCC | | ACAGCCTCGAAAGCACC | | ACGCCTGAGCAGTGGAAG |
| | | | CCGTGCCCTCCAGCAGCTT | | AGACCTACATCTGCAACGT | | CTGACGCTGAGCAAAGC | | TCCCACAGAAGCTACAGC |
| | | | GGGCACCCAGACCTACATC | | GAATCACAAGCCCAGCAAC | | AGACTACGAGAAACACA | | TGCCAGGTCACGCATGAA |
| | | | TGCAACGTGAATCACAAGC | | ACCAAGGTGGACAAGAAA | | AAGTCTACGCCTGCGAA | | GGGAGCACCGTGGAGAAG |
| | | | CCAGCAACACCAAGGTGGA | | GTTGAGCCCAAATCTTGTG | | GTCACCCATCAGGGCCTG | | ACAGTGGCCCCTACAGAA |
| | | | CAAGAAAGTTGAGCCCAAA | | ACAAAACTCACACATGCCC | | AGCTCGCCCGTCACAAA | | TGTTCATAG |
| | | | TCTTGTGACAAAACTCACA | | ACCGTGCCCAGCACCTGAA | | GAGCTTCAACAGGGGAG | | |
| | | | CATGCCCACCGTGCCCAGC | | CTCCTGGGGGGACCGTCAG | | AGTGTTAG | | |
| | | | ACCTGAACTCCTGGGGGGA | | TCTTCCTCTTCCCCCCAAAA | | | | |
| | | | CCGTCAGTCTTCCTCTTCCC | | CCCAAGGACACCCTCTACA | | | | |
| | | | CCCAAAACCCAAGGACACC | | TCACCCGGGAACCTGAGGT | | | | |
| | | | CTCTACATCACCCGGGAAC | | CACATGCGTGGTGGTGGAC | | | | |
| | | | CTGAGGTCACATGCGTGGT | | GTGAGCCACGAAGACCCTG | | | | |
| | | | GGTGGACGTGAGCCACGAA | | AGGTCAAGTTCAACTGGTA | | | | |
| | | | GACCCTGAGGTCAAGTTCA | | CGTGGACGGCGTGGAGGTG | | | | |
| | | | ACTGGTACGTGGACGGCGT | | CATAATGCCAAGACAAAGC | | | | |
| | | | GGAGGTGCATAATGCCAAG | | CGTGCGAGGAGCAGTACGG | | | | |
| | | | ACAAAGCCGTGCGAGGAGC | | CAGCACGTACCGTTGCGTC | | | | |
| | | | AGTACGGCAGCACGTACCG | | AGCGTCCTCACCGTCCTGC | | | | |
| | | | TTGCGTCAGCGTCCTCACC | | ACCAGGACTGGCTGAATGG | | | | |
| | | | GTCCTGCACCAGGACTGGC | | CAAGGAGTACAAGTGCAAG | | | | |
| | | | TGAATGGCAAGGAGTACAA | | GTGTCCAACAAAGCCCTCC | | | | |
| | | | GTGCAAGGTGTCCAACAAA | | CAGCCCCCATCGAGAAAAC | | | | |
| | | | GCCCTCCCAGCCCCCATCG | | CATCTCCAAAGCCAAAGGG | | | | |
| | | | AGAAAACCATCTCCAAAGC | | CAGCCCCGAGAACCACAGG | | | | |
| | | | CAAAGGGCAGCCCCGAGA | | TGTACACCCTGCCCCCATC | | | | |
| | | | ACCACAGGTGTACACCCTG | | CCGGGAGGAGATGACCAA | | | | |
| | | | CCCCCATCCCGGAAGGAGA | | GAACCAGGTCAGCCTGACC | | | | |
| | | | TGACCAAGAACCAGGTCAG | | TGCCTGGTCAAAGGCTTCT | | | | |
| | | | CCTGACCTGCCTGGTCAAA | | ATCCCAGCGACATCGCCGT | | | | |
| | | | GGCTTCTATCCCAGCGACA | | GGAGTGGGAGAGCAATGG | | | | |
| | | | TCGCCGTGGAGTGGGAGAG | | GCAGCCGGAGAACAACTAC | | | | |
| | | | CAATGGGCAGCCGGAGAAC | | GACACCACGCCTCCCGTGC | | | | |
| | | | AACTACAAGACCACGCCTC | | TGGACTCCGACGGCTCCTT | | | | |
| | | | CCGTGCTGAAGTCCGACGG | | CTTCCTCTATAGCGACCTCA | | | | |
| | | | CTCCTTCTTCCTCTATAGCA | | CCGTGGACAAGAGCAGGTG | | | | |
| | | | AGCTCACCGTGGACAAGAG | | GCAGCAGGGGAACGTCTTC | | | | |
| | | | CAGGTGGCAGCAGGGGAA | | TCATGCTCCGTGATGCATG | | | | |
| | | | CGTCTTCTCATGCTCCGTGA | | AGGCTCTGCACAACCACTA | | | | |
| | | | TGCATGAGGCTCTGCACAA | | CACGCAGGACAGCCTCTCC | | | | |
| | | | CCACTACACGCAGAAGAGC | | CTGTCTCCGGGCAAATAG | | | | |
| | | | CTCTCCCTGTCTCCGGGCA | | | | | | |
| | | | AATAG | | | | | | |

Table 14A. Full Length-Nucleic Acid Sequences

| Seq | | |
|---|---|---|
| ID | | |
| No. | Name | Sequence |
| 64 | DLL3_8- | ATGGACATGAGGGTGCCCGCTCAGCTCCTGGGGCTCCTGCTGCTGTGGCTGAGAGGTGCGCGCTGTGAAGTGCAGCT |
| | A7_KDDD huCD3_I2E2_29019_EKK_ | GGTTGAATCTGGCGGCGGATTGGTTCAGCCTGGCGGATCTCTGAAGCTGTCTTGTGCCGCCTCTGGCTTCACCTTCAA |
| | heteroIgG(1zSEFL2v503YTE)_ | CAAATACGCCATCAACTGGGTCCGACAGGCCCCTGGCAAAGGACTGGAATGGGTCGCCCGGATCAGATCCAAGTAC |
| | HC_E | AACAACTACGCTACCTACTACGCCGACGCCGTGAAGGACCGGTTCACCATCTCCAGAGATGACTCCAAGAACACCGT |
| | | CTACCTGCAGATGAACAACCTGAAAACCGAGGACACCGCCGTGTACTACTGTGCGCGGGCCGAGAACATCGGAACC |
| | | TCCTACATCTCCTACTGGGCCTACTGGGGCCAGGGCACACTGGTCACAGTTAGTTCAGCCTCCACCAAGGGCCCATC |
| | | GGTCTTCCCCCTGGCACCCTCCTCCAAGAGCACCTCTGGGGGCACAGCGGCCCTGGGCTGCCTGGTCAAGGACTACT |
| | | TCCCCGAACCGGTGACGGTGTCGTGGAACTCAGGCGCCCTGACCAGCGGCGTGCACACCTTCCCGGCTGTCCTACAG |
| | | TCCTCAGGACTCTACTCCCTCGAGAGCGTGGTGACCGTGCCCTCCAGCAGCTTGGGCACCCAGACCTACATCTGCAA |
| | | CGTGAATCACAAGCCCAGCAACACCAAGGTGGACAAGAAAGTTGAGCCCAAATCTTGTGACAAAACTCACACATGC |
| | | CCACCGTGCCCAGCACCTGAACTCCTGGGGGGACCGTCAGTCTTCCTCTTCCCCCCAAAACCCAAGGACACCCTCTA |
| | | CATCACCCGGGAACCTGAGGTCACATGCGTGGTGGTGGACGTGAGCCACGAAGACCCTGAGGTCAAGTTCAACTGG |
| | | TACGTGGACGGCGTGGAGGTGCATAATGCCAAGACAAAGCCGTGCGAGGAGCAGTACGGCAGCACGTACCGTTGCG |
| | | TCAGCGTCCTCACCGTCCTGCACCAGGACTGGCTGAATGGCAAGGAGTACAAGTGCAAGGTGTCCAACAAAGCCCTC |
| | | CCAGCCCCCATCGAGAAAACCATCTCCAAAGCCAAAGGGCAGCCCCGAGAACCACAGGTGTACACCCTGCCCCCAT |
| | | CCCGGAAGGAGATGACCAAGAACCAGGTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCCTCCCGTGCTGAAGTCCGACGGCTCCTTC |
| | | TTCCTCTATAGCAAGCTCACCGTGGACAAGAGCAGGTGGCAGCAGGGGAACGTCTTCTCATGCTCCGTGATGCATGA |
| | | GGCTCTGCACAACCACTACACGCAGAAGAGCCTCTCCCTGTCTCCGGGCAAATAG |
| 66 | DLL3_8- | ATGGACATGAGGGTGCCCGCTCAGCTCCTGGGGCTCCTGCTGCTGTGGCTGAGAGGTGCGCGCTGTCAAGTCCAACT |
| | A7_KDDD_huCD3_I2E2_29019_EKK_ | TCAAGAATCAGGCCCCGGGCTTGTCAAACCATCTGAAACGCTTTCACTCACTTGTACGGTCAGTGGTGGGTCCATTTC |
| | heteroIgG(1zSEFL2v503YTE)_ | TTCATATTATTGGAGTTGGATTCGGCAACCACCAGGAAAAGGTTTGGAATGGATTGGTTATGTCTATTATTCTGGCAC |
| | HC_K | TACGAATTATAATCCGTCACTTAAATCCCGCGTCACTATTTCCGTCGATACTTCAAAGAATCAATTTAGTCTCAAACT |
| | | TTCCTCAGTCACGGCTGCTGATACTGCTGTTTATTATTGTGCTAGTATTGCAGTCACTGGGTTTTATTTTGATTATTGG |
| | | GGACAAGGTACTCTCGTCACAGTCTCTTCTGCCTCCACCAAGGGCCCATCGGTCTTCCCCCTGGCACCCTCCTCCAAG |
| | | AGCACCTCTGGGGGCACAGCGGCCCTGGGCTGCCTGGTCAAGGACTACTTCCCCGAACCGGTGACGGTGTCGTGGAA |
| | | CTCAGGCGCCCTGACCAGCGGCGTGCACACCTTCCCGGCTGTCCTACAGTCCTCAGGACTCTACTCCCTCAAGAGCG |
| | | TGGTGACCGTGCCCTCCAGCAGCTTGGGCACCCAGACCTACATCTGCAACGTGAATCACAAGCCCAGCAACACCAAG |
| | | GTGGACAAGAAAGTTGAGCCCAAATCTTGTGACAAAACTCACACATGCCCACCGTGCCCAGCACCTGAACTCCTGGG |
| | | GGGACCGTCAGTCTTCCTCTTCCCCCCAAAACCCAAGGACACCCTCTACATCACCCGGGAACCTGAGGTCACATGCG |
| | | TGGTGGTGGACGTGAGCCACGAAGACCCTGAGGTCAAGTTCAACTGGTACGTGGACGGCGTGGAGGTGCATAATGC |
| | | CAAGACAAAGCCGTGCGAGGAGCAGTACGGCAGCACGTACCGTTGCGTCAGCGTCCTCACCGTCCTGCACCAGGAC |
| | | TGGCTGAATGGCAAGGAGTACAAGTGCAAGGTGTCCAACAAAGCCCTCCCAGCCCCCATCGAGAAAACCATCTCCA |
| | | AAGCCAAAGGGCAGCCCCGAGAACCACAGGTGTACACCCTGCCCCCATCCCGGGAGGAGATGACCAAGAACCAGGT |
| | | CAGCCTGACCTGCCTGGTCAAAGGCTTCTATCCCAGCGACATCGCCGTGGAGTGGGAGAGCAATGGGCAGCCGGAG |
| | | AACAACTACGACACCACGCCTCCCGTGCTGGACTCCGACGGCTCCTTCTTCCTCTATAGCGACCTCACCGTGGACAA |
| | | GAGCAGGTGGCAGCAGGGGAACGTCTTCTCATGCTCCGTGATGCATGAGGCTCTGCACAACCACTACACGCAGGAC |
| | | AGCCTCTCCCTGTCTCCGGGCAAATAG |
| 67 | DLL3_8- | AAGATTTTGCAGTTTATTATTGTCAACAATATGATAGATCCCCTCTCACTTTTGGCGGTGGAACTAAACTTGAAATCA |
| | A7_KDDD_huCD3_I2E2_29019_EKK_ | ATGGACATGAGGGTGCCCGCTCAGCTCCTGGGGCTCCTGCTGCTGTGGCTGAGAGGTGCGCGCTGTGAAATTGTACT |
| | heteroIgG(1zSEFL2v503YTE)_ | CACGCAATCACCTGGTACGCTTTCACTCTCCCCTGGTGAACGAGTCACTCTTAGTTGTCGGGCTTCACAACGCGTCAA |
| | LC_E | TAATAATTATCTTGCTTGGTATCAACAACGACCAGGACAAGCTCCACGCTTGCTCATTTATGGTGCAAGTAGTAGAG |
| | | CAACTGGAATTCCTGATCGGTTTTCCGGGTCTGGGAGTGGAACTGATTTTACATTGACGATTTCTCGCCTCGAACCAG |
| | | AACGCACGGTGGCTGCACCATCTGTCTTCATCTTCCCGCCATCTGATGAGCAGTTGAAATCTGGAACTGCCTCTGTTG |
| | | TGTGCCTGCTGAATAACTTCTATCCCAGAGAGGCCAAAGTACAGTGGAAGGTGGATAACGCCCTCCAATCGGGTAAC |
| | | TCCCAGGAGAGTGTCACAGAGCAGGACAGCAAGGACAGCACCTACAGCCTCGAAAGCACCCTGACGCTGAGCAAAG |
| | | CAGACTACGAGAAACACAAAGTCTACGCCTGCGAAGTCACCCATCAGGGCCTGAGCTCGCCCGTCACAAAGAGCTT |
| | | CAACAGGGGAGAGTGTTAG |
| 65 | DLL3_8- | GAGGCTGAGTACTATTGCGTGCTGTGGTACTCCAACAGATGGGTGTTCGGCTCCGGCACCAAGCTGACAGTTCTCGG |
| | A7_KDDD_huCD3_I2E2_29019_EKK_ | ACAGCCCAAGGCTGCACCCTCGGTCACTCTGTTCCCGCCCTCCTCTGAGGAGCTTCAAGCCAACAAGGCCACACTGG |
| | heteroIgG(1zSEFL2v503YTE)_ | ATGGCCTGGGCTCTGCTGCTCCTCACCCTCCTCACTCAGGGCACAGGGTCCTGGGCCCAAACAGTGGTCACCCAAGA |
| | LC_K | GCCTAGCCTGACCGTTTCTCCTGGCGGCACAGTGACCATCACCTGTGGATCTTCTACCGGCGCTGTGACCTCCGGCAA |
| | | CTACCCTAATTGGGTGCAGAAGAAGCCCGGCCAGGCTCCTAGAGGACTGATCGGAGGCACCAAGTTTCTGGCTCCCG |
| | | GCACTCCTGCCAGATTCTCTGGATCTCTGTCCGGCGGAAAGGCCGCTCTGACATTGTCTGGTGTCCAGCCTGAGGAC |
| | | TGTGTCTCATCAGTGACTTCTACCCGGGAGCCGTGACAGTGGCCTGGAAGGCAGATAGCAGCCCCGTCAAGGCGGG |
| | | AGTGGAAACCACCACACCCTCCAAACAAAGCAACAACAAGTACGCGGCCAAGAGCTATCTGAGCCTGACGCCTGAG |
| | | CAGTGGAAGTCCCACAGAAGCTACAGCTGCCAGGTCACGCATGAAGGGAGCACCGTGGAGAAGACAGTGGCCCCTA |
| | | CAGAATGTTCATAG |
| 135 | huDLL3(40861)_huCD3(29019(VH: | CTACCTGCAGATGAACAACCTGAAAACCGAGGACACCGCCGTGTACTACTGTGCGCGGGCCGAGAACATCGGAACC |
| | G110E_F112I_S114T))_heteroIgG4PE- | ATGGACATGAGGGTGCCCGCTCAGCTCCTGGGGCTCCTGCTGCTGTGGCTGAGAGGTGCGCGCTGTGAAGTGCAGCT |
| | FALA-KiH_HC_E | GGTTGAATCTGGCGGCGGATTGGTTCAGCCTGGCGGATCTCTGAAGCTGTCTTGTGCCGCCTCTGGCTTCACCTTCAA |
| | | CAAATACGCCATCAACTGGGTCCGACAGGCCCCTGGCAAAGGACTGGAATGGGTCGCCCGGATCAGATCCAAGTAC |
| | | AACAACTACGCTACCTACTACGCCGACGCCGTGAAGGACCGGTTCACCATCTCCAGAGATGACTCCAAGAACACCGT |
| | | TCCTACATCTCCTACTGGGCCTACTGGGGCCAGGGCACACTGGTCACAGTTAGTTCAGCCTCCACCAAGGGGCCATC |
| | | CGTCTTCCCCCTGGCGCCCTGCTCCAGGAGCACCTCCGAGAGCACAGCCGCCCTGGGCTGCCTGGTCAAGGACTACT |
| | | TCCCCGAACCGGTGACGGTGTCGTGGAACTCAGGCGCCCTGACCAGCGGCGTGCACACCTTCCCGGCTGTCCTACAG |
| | | TCCTCAGGACTCTACTCCCTCGAAAGCGTGGTGACCGTGCCCTCCAGCAGCTTGGGCACGAAGACCTACACCTGCAA |
| | | CGTAGATCACAAGCCCAGCAACACCAAGGTGGACAAGAGAGTTGAGTCCAAATATGGTCCCCCATGCCCACCATGC |
| | | CCAGCACCTGAGGCCGCCGGGGGACCATCAGTCTTCCTGTTCCCCCCAAAACCCAAGGACACTCTCATGATCTCCCG |
| | | GACCCCTGAGGTCACGTGCGTGGTGGTGGACGTGAGCCAGGAAGACCCCGAGGTCCAGTTCAACTGGTACGTGGAT |
| | | GGCGTGGAGGTGCATAATGCCAAGACAAAGCCGCGGGAGGAGCAGTTCAACAGCACGTACCGTGTGGTCAGCGTCC |
| | | TCACCGTCCTGCACCAGGACTGGCTGAACGGCAAGGAGTACAAGTGCAAGGTGTCCAACAAAGGCCTCCCGTCCTCC |
| | | ATCGAGAAAACCATCTCCAAAGCCAAAGGGCAGCCCCGAGAGCCACAGGTGTACACCCTGCCCCCATCCCAGGAGG |
| | | AGATGACCAAGAACCAGGTCAGCCTGAGCTGCGCCGTCAAAGGCTTCTACCCCAGCGACATCGCCGTGGAGTGGGA |
| | | GAGCAATGGGCAGCCGGAGAACAACTACAAGACCACGCCTCCCGTGCTGGACTCCGACGGCTCCTTCTTCCTCGTGA |
| | | GCAGGCTAACCGTGGACAAGAGCAGGTGGCAGGAGGGGAATGTCTTCTCATGCTCCGTGATGCATGAGGCTCTGCA |
| | | CAACCACTACACACAGAAGAGCCTCTCCCTGTCTCTGGGCAAATAG |
| 68 | huDLL3(40861)_huCD3(29019(VH: | CAAGGTGGACAAGAGAGTTGAGTCCAAATATGGTCCCCCATGCCCACCATGCCCAGCACCTGAGGCCGCCGGGGGA |
| | G110E_F112I_S114T))_heteroIgG4PE- | ATGGACATGAGGGTGCCCGCTCAGCTCCTGGGGCTCCTGCTGCTGTGGCTGAGAGGTGCGCGCTGTCAAGTTCAACT |
| | FALA-KiH_HC_K | TGTCGAATCAGGTGGTGGGGCAGTTCAACCAGGGCGCTCATTGCGGTTGTCATGTGCAGCATCAGGTTTTACTTTTAG |
| | | TAATTATGGGATGCATTGGGTTCGCCAAGCACCTGGAAAAGGCCTCGAATGGGTCGCAGTAATTAGTCATCATGGAA |
| | | GTTCCAAATATTATGCAAGAAGCGTCAAAGGTCGCTTTACTATTTCACGCGATAATAGTAAGAACACACTGTATCTT |
| | | GAGATGAATTCACTCAGAGCAGAAGATACTGCTGTCTATTATTGTGCTAGAGATTGGTGGGAACTTGTATTTGATTAT |
| | | TGGGGACAAGGAACGCTCGTCACAGTATCTTCTGCCTCCACCAAGGGGCCATCCGTCTTCCCCCTGGCGCCCTGCTCC |
| | | AGGAGCACCTCCGAGAGCACAGCCGCCCTGGGCTGCCTGGTCAAGGACTACTTCCCCGAACCGGTGACGGTGTCGTG |
| | | GAACTCAGGCGCCCTGACCAGCGGCGTGCACACCTTCCCGGCTGTCCTACAGTCCTCAGGACTCTACTCCCTCAAGA |
| | | GCGTGGTGACCGTGCCCTCCAGCAGCTTGGGCACGAAGACCTACACCTGCAACGTAGATCACAAGCCCAGCAACAC |
| | | CCATCAGTCTTCCTGTTCCCCCCAAAACCCAAGGACACTCTCATGATCTCCCGGACCCCTGAGGTCACGTGCGTGGTG |
| | | GTGGACGTGAGCCAGGAAGACCCCGAGGTCCAGTTCAACTGGTACGTGGATGGCGTGGAGGTGCATAATGCCAAGA |
| | | CAAAGCCGCGGGAGGAGCAGTTCAACAGCACGTACCGTGTGGTCAGCGTCCTCACCGTCCTGCACCAGGACTGGCT |
| | | GAACGGCAAGGAGTACAAGTGCAAGGTGTCCAACAAAGGCCTCCCGTCCTCCATCGAGAAAACCATCTCCAAAGCC |
| | | AAAGGGCAGCCCCGAGAGCCACAGGTGTACACCCTGCCCCCATCCCAGGAGGAGATGACCAAGAACCAGGTCAGCC |
| | | TGTGGTGCCTGGTCAAAGGCTTCTACCCCAGCGACATCGCCGTGGAGTGGGAGAGCAATGGGCAGCCGGAGAACAA |
| | | CTACAAGACCACGCCTCCCGTGCTGGACTCCGACGGCTCCTTCTTCCTCTACAGCAGGCTAACCGTGGACAAGAGCA |
| | | GGTGGCAGGAGGGGAATGTCTTCTCATGCTCCGTGATGCATGAGGCTCTGCACAACCACTACACACAGAAGAGCCTC |
| | | TCCCTGTCTCTGGGCAAATAG |
| 63 | huDLL3(40861)_huCD3(29019(VH: | ATGGACATGAGGGTGCCCGCTCAGCTCCTGGGGCTCCTGCTGCTGTGGCTGAGAGGTGCGCGCTGTGATATTGTTAT |
| | G110E_F112I_S114T))_heteroIgG4PE- | GACACAAACACCTCTTTCACTTTCTGTAACACCGGGACAACCTGCTTCCATTTCTTGTAAATCCAGTCAAAGTCTCTT |
| | FALA-KiH_LC_E | GCATAGTGATGGGAAAACGTTTCTCTATTGGTATTTGCAAAAGCCTGGACAACCACCGCAACTTTTGATTTATGAAG |
| | | TCAGTAATCGCTTTAGTGGAGTTCCAGATCGCTTTTCAGGTTCTGGGTCAGGAACAGATTTTACGCTCAAAATCTCCC |
| | | GGGTTGAAGCTGAAGATGTTGGGGTCTATTATTGTCTTCAAGGGATTCATCTTCCGTTTACTTTTGGGCCAGGGACTA |
| | | AAGTCGAAATTAAACGGACGGTGGCTGCACCATCTGTCTTCATCTTCCCGCCATCTGATGAGCAGTTGAAATCTGGA |
| | | ACTGCCTCTGTTGTGTGCCTGCTGAATAACTTCTATCCCAGAGAGGCCAAAGTACAGTGGAAGGTGGATAACGCCCT |
| | | CCAATCGGGTAACTCCCAGGAGAGTGTCACAGAGCAGGACAGCAAGGACAGCACCTACAGCCTCGAAAGCACCCTG |
| | | ACGCTGAGCAAAGCAGACTACGAGAAACACAAAGTCTACGCCTGCGAAGTCACCCATCAGGGCCTGAGCTCGCCCG |
| | | TCACAAAGAGCTTCAACAGGGGAGAGTGTTAG |
| 65 | huDLL3(40861)_huCD3(29019(VH: | ATGGCCTGGGCTCTGCTGCTCCTCACCCTCCTCACTCAGGGCACAGGGTCCTGGGCCCAAACAGTGGTCACCCAAGA |
| | G110E_F112I_S114T))_heteroIgG4PE- | GCCTAGCCTGACCGTTTCTCCTGGCGGCACAGTGACCATCACCTGTGGATCTTCTACCGGCGCTGTGACCTCCGGCAA |
| | FALA-KiH_LC_K | CTACCCTAATTGGGTGCAGAAGAAGCCCGGCCAGGCTCCTAGAGGACTGATCGGAGGCACCAAGTTTCTGGCTCCCG |
| | | GCACTCCTGCCAGATTCTCTGGATCTCTGTCCGGCGGAAAGGCCGCTCTGACATTGTCTGGTGTCCAGCCTGAGGAC |
| | | GAGGCTGAGTACTATTGCGTGCTGTGGTACTCCAACAGATGGGTGTTCGGCTCCGGCACCAAGCTGACAGTTCTCGG |
| | | ACAGCCCAAGGCTGCACCCTCGGTCACTCTGTTCCCGCCCTCCTCTGAGGAGCTTCAAGCCAACAAGGCCACACTGG |
| | | TGTGTCTCATCAGTGACTTCTACCCGGGAGCCGTGACAGTGGCCTGGAAGGCAGATAGCAGCCCCGTCAAGGCGGG |
| | | AGTGGAAACCACCACACCCTCCAAACAAAGCAACAACAAGTACGCGGCCAAGAGCTATCTGAGCCTGACGCCTGAG |
| | | CAGTGGAAGTCCCACAGAAGCTACAGCTGCCAGGTCACGCATGAAGGGAGCACCGTGGAGAAGACAGTGGCCCCTA |
| | | CAGAATGTTCATAG |
| 64 | DLL3_0- | ATGGACATGAGGGTGCCCGCTCAGCTCCTGGGGCTCCTGCTGCTGTGGCTGAGAGGTGCGCGCTGTGAAGTGCAGCT |
| | G4_KDDD huCD3_I2E2_29019_EKK_ | GGTTGAATCTGGCGGCGGATTGGTTCAGCCTGGCGGATCTCTGAAGCTGTCTTGTGCCGCCTCTGGCTTCACCTTCAA |
| | heteroIgG(1zSEFL2v503YTE)_ | CAAATACGCCATCAACTGGGTCCGACAGGCCCCTGGCAAAGGACTGGAATGGGTCGCCCGGATCAGATCCAAGTAC |
| | HC_E | AACAACTACGCTACCTACTACGCCGACGCCGTGAAGGACCGGTTCACCATCTCCAGAGATGACTCCAAGAACACCGT |
| | | CTACCTGCAGATGAACAACCTGAAAACCGAGGACACCGCCGTGTACTACTGTGCGCGGGCCGAGAACATCGGAACC |
| | | TCCTACATCTCCTACTGGGCCTACTGGGGCCAGGGCACACTGGTCACAGTTAGTTCAGCCTCCACCAAGGGCCCATC |
| | | GGTCTTCCCCCTGGCACCCTCCTCCAAGAGCACCTCTGGGGGCACAGCGGCCCTGGGCTGCCTGGTCAAGGACTACT |
| | | TCCCCGAACCGGTGACGGTGTCGTGGAACTCAGGCGCCCTGACCAGCGGCGTGCACACCTTCCCGGCTGTCCTACAG |
| | | TCCTCAGGACTCTACTCCCTCGAGAGCGTGGTGACCGTGCCCTCCAGCAGCTTGGGCACCCAGACCTACATCTGCAA |
| | | CGTGAATCACAAGCCCAGCAACACCAAGGTGGACAAGAAAGTTGAGCCCAAATCTTGTGACAAAACTCACACATGC |
| | | CCACCGTGCCCAGCACCTGAACTCCTGGGGGGACCGTCAGTCTTCCTCTTCCCCCCAAAACCCAAGGACACCCTCTA |
| | | CATCACCCGGGAACCTGAGGTCACATGCGTGGTGGTGGACGTGAGCCACGAAGACCCTGAGGTCAAGTTCAACTGG |
| | | TACGTGGACGGCGTGGAGGTGCATAATGCCAAGACAAAGCCGTGCGAGGAGCAGTACGGCAGCACGTACCGTTGCG |
| | | TCAGCGTCCTCACCGTCCTGCACCAGGACTGGCTGAATGGCAAGGAGTACAAGTGCAAGGTGTCCAACAAAGCCCTC |
| | | CCAGCCCCCATCGAGAAAACCATCTCCAAAGCCAAAGGGCAGCCCCGAGAACCACAGGTGTACACCCTGCCCCCAT |
| | | CCCGGAAGGAGATGACCAAGAACCAGGTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCCTCCCGTGCTGAAGTCCGACGGCTCCTTC |
| | | TTCCTCTATAGCAAGCTCACCGTGGACAAGAGCAGGTGGCAGCAGGGGAACGTCTTCTCATGCTCCGTGATGCATGA |
| | | GGCTCTGCACAACCACTACACGCAGAAGAGCCTCTCCCTGTCTCCGGGCAAATAG |
| 62 | DLL3_0- | GAGATGAATTCACTCAGAGCAGAAGATACTGCTGTCTATTATTGTGCTAGAGATTGGTGGGAACTTGTATTTGATTAT |
| | G4_KDDD huCD3_I2E2_29019_EKK_ | TGGGGACAAGGAACGCTCGTCACAGTATCTTCTGCCTCCACCAAGGGCCCATCGGTCTTCCCCCTGGCACCCTCCTCC |
| | heteroIgG(1zSEFL2v503YTE)_ | ATGGACATGAGGGTGCCCGCTCAGCTCCTGGGGCTCCTGCTGCTGTGGCTGAGAGGTGCGCGCTGTCAAGTTCAACT |
| | HC_K | TGTCGAATCAGGTGGTGGGGCAGTTCAACCAGGGCGCTCATTGCGGTTGTCATGTGCAGCATCAGGTTTTACTTTTAG |
| | | TAATTATGGGATGCATTGGGTTCGCCAAGCACCTGGAAAAGGCCTCGAATGGGTCGCAGTAATTAGTCATCATGGAA |
| | | GTTCCAAATATTATGCAAGAAGCGTCAAAGGTCGCTTTACTATTTCACGCGATAATAGTAAGAACACACTGTATCTT |
| | | AAGAGCACCTCTGGGGGCACAGCGGCCCTGGGCTGCCTGGTCAAGGACTACTTCCCCGAACCGGTGACGGTGTCGTG |
| | | GAACTCAGGCGCCCTGACCAGCGGCGTGCACACCTTCCCGGCTGTCCTACAGTCCTCAGGACTCTACTCCCTCAAGA |
| | | GCGTGGTGACCGTGCCCTCCAGCAGCTTGGGCACCCAGACCTACATCTGCAACGTGAATCACAAGCCCAGCAACACC |
| | | AAGGTGGACAAGAAAGTTGAGCCCAAATCTTGTGACAAAACTCACACATGCCCACCGTGCCCAGCACCTGAACTCCT |
| | | GGGGGGACCGTCAGTCTTCCTCTTCCCCCCAAAACCCAAGGACACCCTCTACATCACCCGGGAACCTGAGGTCACAT |
| | | GCGTGGTGGTGGACGTGAGCCACGAAGACCCTGAGGTCAAGTTCAACTGGTACGTGGACGGCGTGGAGGTGCATAA |
| | | TGCCAAGACAAAGCCGTGCGAGGAGCAGTACGGCAGCACGTACCGTTGCGTCAGCGTCCTCACCGTCCTGCACCAG |
| | | GACTGGCTGAATGGCAAGGAGTACAAGTGCAAGGTGTCCAACAAAGCCCTCCCAGCCCCCATCGAGAAAACCATCT |
| | | CCAAAGCCAAAGGGCAGCCCCGAGAACCACAGGTGTACACCCTGCCCCCATCCCGGGAGGAGATGACCAAGAACCA |
| | | GGTCAGCCTGACCTGCCTGGTCAAAGGCTTCTATCCCAGCGACATCGCCGTGGAGTGGGAGAGCAATGGGCAGCCG |
| | | GAGAACAACTACGACACCACGCCTCCCGTGCTGGACTCCGACGGCTCCTTCTTCCTCTATAGCGACCTCACCGTGGA |
| | | CAAGAGCAGGTGGCAGCAGGGGAACGTCTTCTCATGCTCCGTGATGCATGAGGCTCTGCACAACCACTACACGCAG |
| | | GACAGCCTCTCCCTGTCTCCGGGCAAATAG |
| 63 | DLL3_0- | GGGTTGAAGCTGAAGATGTTGGGGTCTATTATTGTCTTCAAGGGATTCATCTTCCGTTTACTTTTGGGCCAGGGACTA |
| | G4_KDDD huCD3_I2E2_29019_EKK_ | AAGTCGAAATTAAACGGACGGTGGCTGCACCATCTGTCTTCATCTTCCCGCCATCTGATGAGCAGTTGAAATCTGGA |
| | heteroIgG(1zSEFL2v503YTE)_ | ATGGACATGAGGGTGCCCGCTCAGCTCCTGGGGCTCCTGCTGCTGTGGCTGAGAGGTGCGCGCTGTGATATTGTTAT |
| | LC_E | GACACAAACACCTCTTTCACTTTCTGTAACACCGGGACAACCTGCTTCCATTTCTTGTAAATCCAGTCAAAGTCTCTT |
| | | GCATAGTGATGGGAAAACGTTTCTCTATTGGTATTTGCAAAAGCCTGGACAACCACCGCAACTTTTGATTTATGAAG |
| | | TCAGTAATCGCTTTAGTGGAGTTCCAGATCGCTTTTCAGGTTCTGGGTCAGGAACAGATTTTACGCTCAAAATCTCCC |
| | | ACTGCCTCTGTTGTGTGCCTGCTGAATAACTTCTATCCCAGAGAGGCCAAAGTACAGTGGAAGGTGGATAACGCCCT |
| | | CCAATCGGGTAACTCCCAGGAGAGTGTCACAGAGCAGGACAGCAAGGACAGCACCTACAGCCTCGAAAGCACCCTG |
| | | ACGCTGAGCAAAGCAGACTACGAGAAACACAAAGTCTACGCCTGCGAAGTCACCCATCAGGGCCTGAGCTCGCCCG |
| | | TCACAAAGAGCTTCAACAGGGGAGAGTGTTAG |
| 65 | DLL3_0- | GAGGCTGAGTACTATTGCGTGCTGTGGTACTCCAACAGATGGGTGTTCGGCTCCGGCACCAAGCTGACAGTTCTCGG |
| | G4_KDDD_huCD3_I2E2_29019_EKK_ | ATGGCCTGGGCTCTGCTGCTCCTCACCCTCCTCACTCAGGGCACAGGGTCCTGGGCCCAAACAGTGGTCACCCAAGA |
| | heteroIgG(1zSEFL2v503YTE)_ | GCCTAGCCTGACCGTTTCTCCTGGCGGCACAGTGACCATCACCTGTGGATCTTCTACCGGCGCTGTGACCTCCGGCAA |
| | LC_K | CTACCCTAATTGGGTGCAGAAGAAGCCCGGCCAGGCTCCTAGAGGACTGATCGGAGGCACCAAGTTTCTGGCTCCCG |
| | | GCACTCCTGCCAGATTCTCTGGATCTCTGTCCGGCGGAAAGGCCGCTCTGACATTGTCTGGTGTCCAGCCTGAGGAC |
| | | ACAGCCCAAGGCTGCACCCTCGGTCACTCTGTTCCCGCCCTCCTCTGAGGAGCTTCAAGCCAACAAGGCCACACTGG |
| | | TGTGTCTCATCAGTGACTTCTACCCGGGAGCCGTGACAGTGGCCTGGAAGGCAGATAGCAGCCCCGTCAAGGCGGG |
| | | AGTGGAAACCACCACACCCTCCAAACAAAGCAACAACAAGTACGCGGCCAAGAGCTATCTGAGCCTGACGCCTGAG |
| | | CAGTGGAAGTCCCACAGAAGCTACAGCTGCCAGGTCACGCATGAAGGGAGCACCGTGGAGAAGACAGTGGCCCCTA |
| | | CAGAATGTTCATAG |
| 134 | DLL3_8-A7 | ATGGACATGAGGGTGCCCGCTCAGCTCCTGGGGCTCCTGCTGCTGTGGCTGAGAGGTGCGCGCTGTGAAGTGCAGCT |
| | KDDD_huCD3_29019_EKK_ | GGTTGAATCTGGCGGCGGATTGGTTCAGCCTGGCGGATCTCTGAAGCTGTCTTGTGCCGCCTCTGGCTTCACCTTCAA |
| | heteroIgG(1zSEFL2v503YTE)_HC_E | CAAATACGCCATCAACTGGGTCCGACAGGCCCCTGGCAAAGGACTGGAATGGGTCGCCCGGATCAGATCCAAGTAC |
| | | AACAACTACGCTACCTACTACGCCGACGCCGTGAAGGACCGGTTCACCATCTCCAGAGATGACTCCAAGAACACCGT |
| | | GTACCTGCAGATGAACAACCTCAAGACCGAGGACACCGCCGTGTACTACTGTGCCAGAGCCGGCAACTTCGGCTCCT |
| | | CCTACATCAGCTACTGGGCCTATTGGGGCCAGGGCACACTGGTCACAGTTAGTTCAGCCTCCACCAAGGGCCCATCG |
| | | GTCTTCCCCCTGGCACCCTCCTCCAAGAGCACCTCTGGGGGCACAGCGGCCCTGGGCTGCCTGGTCAAGGACTACTT |
| | | CCCCGAACCGGTGACGGTGTCGTGGAACTCAGGCGCCCTGACCAGCGGCGTGCACACCTTCCCGGCTGTCCTACAGT |
| | | CCTCAGGACTCTACTCCCTCGAGAGCGTGGTGACCGTGCCCTCCAGCAGCTTGGGCACCCAGACCTACATCTGCAAC |
| | | GTGAATCACAAGCCCAGCAACACCAAGGTGGACAAGAAAGTTGAGCCCAAATCTTGTGACAAAACTCACACATGCC |
| | | CACCGTGCCCAGCACCTGAACTCCTGGGGGGACCGTCAGTCTTCCTCTTCCCCCCAAAACCCAAGGACACCCTCTAC |
| | | ATCACCCGGGAACCTGAGGTCACATGCGTGGTGGTGGACGTGAGCCACGAAGACCCTGAGGTCAAGTTCAACTGGT |
| | | ACGTGGACGGCGTGGAGGTGCATAATGCCAAGACAAAGCCGTGCGAGGAGCAGTACGGCAGCACGTACCGTTGCGT |
| | | CAGCGTCCTCACCGTCCTGCACCAGGACTGGCTGAATGGCAAGGAGTACAAGTGCAAGGTGTCCAACAAAGCCCTC |
| | | CCAGCCCCCATCGAGAAAACCATCTCCAAAGCCAAAGGGCAGCCCCGAGAACCACAGGTGTACACCCTGCCCCCAT |
| | | CCCGGAAGGAGATGACCAAGAACCAGGTCAGCCTGACCTGCCTGGTCAAAGGCTTCTATCCCAGCGACATCGCCGT |
| | | GGAGTGGGAGAGCAATGGGCAGCCGGAGAACAACTACAAGACCACGCCTCCCGTGCTGAAGTCCGACGGCTCCTTC |
| | | TTCCTCTATAGCAAGCTCACCGTGGACAAGAGCAGGTGGCAGCAGGGGAACGTCTTCTCATGCTCCGTGATGCATGA |
| | | GGCTCTGCACAACCACTACACGCAGAAGAGCCTCTCCCTGTCTCCGGGCAAATAG |
| 66 | DLL3_8-A7 | GTGGACAAGAAAGTTGAGCCCAAATCTTGTGACAAAACTCACACATGCCCACCGTGCCCAGCACCTGAACTCCTGGG |
| | KDDD_huCD3_29019_EKK_ | ATGGACATGAGGGTGCCCGCTCAGCTCCTGGGGCTCCTGCTGCTGTGGCTGAGAGGTGCGCGCTGTCAAGTCCAACT |
| | heteroIgG(1zSEFL2v503YTE)_HC_K | TCAAGAATCAGGCCCCGGGCTTGTCAAACCATCTGAAACGCTTTCACTCACTTGTACGGTCAGTGGTGGGTCCATTTC |
| | | TTCATATTATTGGAGTTGGATTCGGCAACCACCAGGAAAAGGTTTGGAATGGATTGGTTATGTCTATTATTCTGGCAC |
| | | TACGAATTATAATCCGTCACTTAAATCCCGCGTCACTATTTCCGTCGATACTTCAAAGAATCAATTTAGTCTCAAACT |
| | | TTCCTCAGTCACGGCTGCTGATACTGCTGTTTATTATTGTGCTAGTATTGCAGTCACTGGGTTTTATTTTGATTATTGG |
| | | GGACAAGGTACTCTCGTCACAGTCTCTTCTGCCTCCACCAAGGGCCCATCGGTCTTCCCCCTGGCACCCTCCTCCAAG |
| | | AGCACCTCTGGGGGCACAGCGGCCCTGGGCTGCCTGGTCAAGGACTACTTCCCCGAACCGGTGACGGTGTCGTGGAA |
| | | CTCAGGCGCCCTGACCAGCGGCGTGCACACCTTCCCGGCTGTCCTACAGTCCTCAGGACTCTACTCCCTCAAGAGCG |
| | | TGGTGACCGTGCCCTCCAGCAGCTTGGGCACCCAGACCTACATCTGCAACGTGAATCACAAGCCCAGCAACACCAAG |
| | | GGGACCGTCAGTCTTCCTCTTCCCCCCAAAACCCAAGGACACCCTCTACATCACCCGGGAACCTGAGGTCACATGCG |
| | | TGGTGGTGGACGTGAGCCACGAAGACCCTGAGGTCAAGTTCAACTGGTACGTGGACGGCGTGGAGGTGCATAATGC |
| | | CAAGACAAAGCCGTGCGAGGAGCAGTACGGCAGCACGTACCGTTGCGTCAGCGTCCTCACCGTCCTGCACCAGGAC |
| | | TGGCTGAATGGCAAGGAGTACAAGTGCAAGGTGTCCAACAAAGCCCTCCCAGCCCCCATCGAGAAAACCATCTCCA |
| | | AAGCCAAAGGGCAGCCCCGAGAACCACAGGTGTACACCCTGCCCCCATCCCGGGAGGAGATGACCAAGAACCAGGT |
| | | CAGCCTGACCTGCCTGGTCAAAGGCTTCTATCCCAGCGACATCGCCGTGGAGTGGGAGAGCAATGGGCAGCCGGAG |
| | | AACAACTACGACACCACGCCTCCCGTGCTGGACTCCGACGGCTCCTTCTTCCTCTATAGCGACCTCACCGTGGACAA |
| | | GAGCAGGTGGCAGCAGGGGAACGTCTTCTCATGCTCCGTGATGCATGAGGCTCTGCACAACCACTACACGCAGGAC |
| | | AGCCTCTCCCTGTCTCCGGGCAAATAG |
| 67 | DLL3_8-A7 | ATGGACATGAGGGTGCCCGCTCAGCTCCTGGGGCTCCTGCTGCTGTGGCTGAGAGGTGCGCGCTGTGAAATTGTACT |
| | KDDD_huCD3_29019_EKK_heteroIgG | CACGCAATCACCTGGTACGCTTTCACTCTCCCCTGGTGAACGAGTCACTCTTAGTTGTCGGGCTTCACAACGCGTCAA |
| | (1zSEFL2v503YTE)_LC E | TAATAATTATCTTGCTTGGTATCAACAACGACCAGGACAAGCTCCACGCTTGCTCATTTATGGTGCAAGTAGTAGAG |
| | | CAACTGGAATTCCTGATCGGTTTTCCGGGTCTGGGAGTGGAACTGATTTTACATTGACGATTTCTCGCCTCGAACCAG |
| | | AAGATTTTGCAGTTTATTATTGTCAACAATATGATAGATCCCCTCTCACTTTTGGCGGTGGAACTAAACTTGAAATCA |
| | | AACGCACGGTGGCTGCACCATCTGTCTTCATCTTCCCGCCATCTGATGAGCAGTTGAAATCTGGAACTGCCTCTGTTG |
| | | TGTGCCTGCTGAATAACTTCTATCCCAGAGAGGCCAAAGTACAGTGGAAGGTGGATAACGCCCTCCAATCGGGTAAC |
| | | TCCCAGGAGAGTGTCACAGAGCAGGACAGCAAGGACAGCACCTACAGCCTCGAAAGCACCCTGACGCTGAGCAAAG |
| | | CAGACTACGAGAAACACAAAGTCTACGCCTGCGAAGTCACCCATCAGGGCCTGAGCTCGCCCGTCACAAAGAGCTT |
| | | CAACAGGGGAGAGTGTTAG |
| 70 | DLL3_8-A7 | ATGGCCTGGGCTCTGCTGCTCCTCACCCTCCTCACTCAGGGCACAGGGTCCTGGGCCCAAACAGTGGTCACCCAAGA |
| | KDDD_huCD3_29019_EKK_heteroIgG | GCCTAGCCTGACCGTTTCTCCTGGCGGCACCGTGACCATCACCTGTGGATCTTCTACCGGCGCTGTGACCTCCGGCAA |
| | (1zSEFL2v503YTE)_LC_K | CTACCCTAATTGGGTGCAGAAGAAGCCCGGCCAGGCTCCTAGAGGACTGATCGGAGGCACCAAGTTTCTGGCTCCCG |
| | | GCACTCCTGCCAGATTCTCCGGTTCTCTGTCTGGCGGAAAGGCCGCTCTGACATTGTCTGGCGTGCAGCCTGAGGATG |
| | | AGGCTGAGTACTATTGCGTGCTGTGGTACTCCAACAGATGGGTGTTCGGCTCCGGCACCAAGCTGACAGTTCTCGGG |
| | | CAGCCCAAGGCTGCACCCTCGGTCACTCTGTTCCCGCCCTCCTCTGAGGAGCTTCAAGCCAACAAGGCCACACTGGT |
| | | GTGTCTCATCAGTGACTTCTACCCGGGAGCCGTGACAGTGGCCTGGAAGGCAGATAGCAGCCCCGTCAAGGCGGGA |
| | | GTGGAAACCACCACACCCTCCAAACAAAGCAACAACAAGTACGCGGCCAAGAGCTATCTGAGCCTGACGCCTGAGC |
| | | AGTGGAAGTCCCACAGAAGCTACAGCTGCCAGGTCACGCATGAAGGGAGCACCGTGGAGAAGACAGTGGCCCCTAC |
| | | AGAATGTTCATAG |

1. A heteromultimer comprising a first heterodimer that binds to human delta-like ligand 3 (DLL3) and a second heterodimer that binds to human cluster of differentiation (CD) 3 (CD3), wherein:
(a) the first heterodimer comprises: a heavy chain (HC) comprising the amino acid sequence of SEQ ID NO: 54 or SEQ ID NO: 58; and a light chain (LC) comprising the amino acid sequence of SEQ ID NO: 55 or SEQ ID NO: 59; and
(b) the second heterodimer comprises: a heavy chain comprising the amino acid sequence of SEQ ID NO: 56 or SEQ ID NO: 132; and a light chain comprising the amino acid sequence of SEQ ID NO: 57.

2. The heteromultimer of claim 1, wherein the first heterodimer comprises a heavy chain comprising the amino acid sequence of SEQ ID NO: 54 and a light chain comprising the amino acid sequence of SEQ ID NO: 55.

3. The heteromultimer of claim 1, wherein the first heterodimer comprises a heavy chain comprising the amino acid sequence of SEQ ID NO: 58 and a light chain comprising the amino acid sequence of SEQ ID NO: 59.

4. The heteromultimer of claim 1, wherein the second heterodimer comprises a heavy chain comprising the amino acid sequence of SEQ ID NO: 56 and a light chain comprising the amino acid sequence of SEQ ID NO: 57.

5. The heteromultimer of claim 1, wherein the second heterodimer comprises a heavy chain comprising the amino acid sequence of SEQ ID NO: 132 and a light chain comprising the amino acid sequence of SEQ ID NO: 57.

6. The heteromultimer of claim 1, wherein:
(a) the first heterodimer comprises a heavy chain comprising the amino acid sequence of SEQ ID NO: 54 and a light chain comprising the amino acid sequence of SEQ ID NO: 55; and
(b) the second heterodimer comprises a heavy chain comprising the amino acid sequence of SEQ ID NO: 56 and a light chain comprising the amino acid sequence of SEQ ID NO: 57.

7. The heteromultimer of claim 1, wherein:
(a) the first heterodimer comprises a heavy chain comprising the amino acid sequence of SEQ ID NO: 54 and a light chain comprising the amino acid sequence of SEQ ID NO: 55; and
(b) the second heterodimer comprises a heavy chain comprising the amino acid sequence of SEQ ID NO: 132 and a light chain comprising the amino acid sequence of SEQ ID NO: 57.

8. The heteromultimer of claim 1, wherein:
(a) the first heterodimer comprises a heavy chain comprising the amino acid sequence of SEQ ID NO: 58 and a light chain comprising the amino acid sequence of SEQ ID NO: 59; and
(b) the second heterodimer comprises a heavy chain comprising the amino acid sequence of SEQ ID NO: 56 and a light chain comprising the amino acid sequence of SEQ ID NO: 57.

9. The heteromultimer of claim 1, wherein:
(a) the first heterodimer comprises a heavy chain comprising the amino acid sequence of SEQ ID NO: 58 and a light chain comprising the amino acid sequence of SEQ ID NO: 59; and
(b) the second heterodimer comprises a heavy chain comprising the amino acid sequence of SEQ ID NO: 132 and a light chain comprising the amino acid sequence of SEQ ID NO: 57.

10. The heteromultimer of claim 1, wherein the first heterodimer binds to human DLL3 expressed on the surface of a target cell.

11. The heteromultimer of claim 10, wherein the target cell is a cancer cell.

12. The heteromultimer of claim 11, wherein the cancer cell is a neuroendocrine cancer cell.

13. The heteromultimer of claim 12, wherein the neuroendocrine cancer is small cell lung cancer (SCLC) or neuroendocrine prostate cancer (NEPC).

14. The heteromultimer of claim 1, wherein the second heterodimer binds to human CD3 expressed on the surface of a T cell.

15. A composition comprising the heteromultimer of claim 1 and a pharmaceutically acceptable carrier.

16.-18. (canceled)

19. A kit comprising the composition of claim 15 and instructions for use.

20. A method of inhibiting growth of DLL3-expressing cancer cells, which comprises contacting a population of DLL3-expressing cancer cells and CD3-expressing T cells with an effective amount of the heteromultimer of claim 1.

21. The method of claim 20, wherein the population of DLL3-expressing cancer cells and CD3-expressing T cells are in vitro.

22. The method of claim 20, wherein the population of DLL3-expressing cancer cells and CD3-expressing T cells are in vivo.

23. A method of treating a DLL3-expressing cancer in a subject in need thereof, which comprises administering to the subject an effective amount of the heteromultimer of claim 1.

24. The method of claim 23, wherein the cancer is a neuroendocrine cancer.

25. The method of claim 24, wherein the neuroendocrine cancer is small cell lung cancer (SCLC) or neuroendocrine prostate cancer (NEPC).

26. The method of claim 23, wherein the heteromultimer or composition is administered to the subject intravenously, intramuscularly, or subcutaneously.

27. The method of claim 23, which further comprises administering at least one additional therapeutic agent to the subject.

28. The method of claim 27, wherein the additional therapeutic agent comprises one or more chemotherapeutic agents or a programmed cell death 1 (PD-1)/programmed cell death ligand 1 (PD-L1) antagonist.

29. The method of claim 28, wherein the PD-1/PD-L1 antagonist is an anti-PD-1 antibody or an anti-PD-L1 antibody.

30. The method of claim 29, wherein the anti-PD-1 antibody comprises nivolumab, pembrolizumab, or cemiplimab.

31. The method of claim 29, wherein the anti-PD-L1 antibody comprises atezolizumab, avelumab, or durvalumab.

32. The method of claim 28, wherein the one or more chemotherapeutic agents comprises an alkylating agent, a platinum-based chemotherapeutic agent, etoposide, or any combination thereof.

33. The method of claim 32, wherein the alkylating agent is lurbinectedin.

34. The method of claim 32, wherein the platinum-based chemotherapeutic agent is carboplatin or cisplatin.

35. The method of claim 23, wherein the subject is a human.

36. A nucleic acid sequence encoding the heteromultimer of claim 1.

37.-39. (canceled)

40. A method of inhibiting growth of DLL3-expressing cancer cells, which comprises contacting a population of DLL3-expressing cancer cells and CD3-expressing T cells with an effective amount of the composition of claim 15.

41. A method of treating a DLL3-expressing cancer in a subject in need thereof, which comprises administering to the subject an effective amount of the composition of claim 15.

42. A composition comprising a heteromultimer and a pharmaceutically acceptable carrier, wherein the heteromultimer comprises (a) a first heterodimer comprising a heavy chain amino acid sequence of SEQ ID NO: 54 and a light chain amino acid sequence of SEQ ID NO: 55; and (b) a second heterodimer comprising a heavy chain amino acid sequence of SEQ ID NO: 56 and a light chain amino acid sequence of SEQ ID NO: 57.

43. A method of inhibiting growth of DLL3-expressing cancer cells, which comprises contacting a population of DLL3-expressing cancer cells and CD3-expressing T cells with an effective amount of the composition of claim 42.

44. A method of treating a DLL3-expressing cancer in a subject in need thereof, which comprises administering to the subject an effective amount of the composition of claim 42.


SEQUENCE LISTING (135 sequences)

SEQ ID NO: 1 - protein, 5 aa, synthetic construct
NYGMH

SEQ ID NO: 2 - protein, 17 aa, synthetic construct
VISHHGSSKYYARSVKG

SEQ ID NO: 3 - protein, 9 aa, synthetic construct
DWWELVFDY

SEQ ID NO: 4 - protein, 16 aa, synthetic construct
KSSQSLLHSDGKTFLY

SEQ ID NO: 5 - protein, 7 aa, synthetic construct
EVSNRFS

SEQ ID NO: 6 - protein, 9 aa, synthetic construct
LQGIHLPFT

SEQ ID NO: 7 - protein, 5 aa, synthetic construct
KYAIN

SEQ ID NO: 8 - protein, 19 aa, synthetic construct
RIRSKYNNYATYYADAVKD

SEQ ID NO: 9 - protein, 14 aa, synthetic construct
AENIGTSYISYWAY

SEQ ID NO: 10 - protein, 14 aa, synthetic construct
GSSTGAVTSGNYPN

SEQ ID NO: 11 - protein, 7 aa, synthetic construct
GTKFLAP

SEQ ID NO: 12 - protein, 9 aa, synthetic construct
VLWYSNRWV

SEQ ID NO: 13 - protein, 5 aa, synthetic construct
SYYWS

SEQ ID NO: 14 - protein, 16 aa, synthetic construct
YVYYSGTTNYNPSLKS

SEQ ID NO: 15 - protein, 10 aa, synthetic construct
IAVTGFYFDY

SEQ ID NO: 16 - protein, 12 aa, synthetic construct
RASQRVNNNYLA

SEQ ID NO: 17 - protein, 7 aa, synthetic construct
GASSRAT

SEQ ID NO: 18 - protein, 9 aa, synthetic construct
QQYDRSPLT

SEQ ID NO: 19 - protein, 14 aa, synthetic construct
AGNFGSSYISYWAY

SEQ ID NO: 20 - protein, 30 aa, synthetic construct
QVQLVESGGGAVQPGRSLRLSCAASGFTFS

SEQ ID NO: 21 - protein, 14 aa, synthetic construct
WVRQAPGKGLEWVA

SEQ ID NO: 22 - protein, 32 aa, synthetic construct
RFTISRDNSKNTLYLEMNSLRAEDTAVYYCAR

SEQ ID NO: 23 - protein, 11 aa, synthetic construct
WGQGTLVTVSS

SEQ ID NO: 24 - protein, 23 aa, synthetic construct
DIVMTQTPLSLSVTPGQPASISC

SEQ ID NO: 25 - protein, 15 aa, synthetic construct
WYLQKPGQPPQLLIY

SEQ ID NO: 26 - protein, 32 aa, synthetic construct
GVPDRFSGSGSGTDFTLKISRVEAEDVGVYYC

SEQ ID NO: 27 - protein, 11 aa, synthetic construct
FGPGTKVEIKR

SEQ ID NO: 28 - protein, 30 aa, synthetic construct
EVQLVESGGGLVQPGGSLKLSCAASGFTFN

SEQ ID NO: 29 - protein, 32 aa, synthetic construct
RFTISRDDSKNTVYLQMNNLKTEDTAVYYCAR

SEQ ID NO: 30 - protein, 22 aa, synthetic construct
QTVVTQEPSLTVSPGGTVTITC

SEQ ID NO: 31 - protein, 15 aa, synthetic construct
WVQKKPGQAPRGLIG

SEQ ID NO: 32 - protein, 32 aa, synthetic construct
GTPARFSGSLSGGKAALTLSGVQPEDEAEYYC

SEQ ID NO: 33 - protein, 11 aa, synthetic construct
FGSGTKLTVLG

SEQ ID NO: 34 - protein, 30 aa, synthetic construct
QVQLQESGPGLVKPSETLSLTCTVSGGSIS

SEQ ID NO: 35 - protein, 14 aa, synthetic construct
WIRQPPGKGLEWIG

SEQ ID NO: 36 - protein, 32 aa, synthetic construct
RVTISVDTSKNQFSLKLSSVTAADTAVYYCAS

SEQ ID NO: 37 - protein, 23 aa, synthetic construct
EIVLTQSPGTLSLSPGERVTLSC

SEQ ID NO: 38 - protein, 15 aa, synthetic construct
WYQQRPGQAPRLLIY

SEQ ID NO: 39 - protein, 32 aa, synthetic construct
GIPDRFSGSGSGTDFTLTISRLEPEDFAVYYC

SEQ ID NO: 40 - protein, 11 aa, synthetic construct
FGGGTKLEIKR

SEQ ID NO: 41 - protein, 118 aa, synthetic construct
QVQLVESGGGAVQPGRSLRLSCAASGFTFSNYGMHWVRQAPGKGLEWVAVISHHGSSKYYARSVKGRFTISRDNSKNTLYLEMNSLRAEDTAVYYCARDWWELVFDYWGQGTLVTVSS

SEQ ID NO: 42 - protein, 113 aa, synthetic construct
DIVMTQTPLSLSVTPGQPASISCKSSQSLLHSDGKTFLYWYLQKPGQPPQLLIYEVSNRFSGVPDRFSGSGSGTDFTLKISRVEAEDVGVYYCLQGIHLPFTFGPGTKVEIKR

SEQ ID NO: 43 - protein, 125 aa, synthetic construct
EVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAENIGTSYISYWAYWGQGTLVTVSS

SEQ ID NO: 44 - protein, 110 aa, synthetic construct
QTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVLG

SEQ ID NO: 45 - protein, 118 aa, synthetic construct
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 46 - protein, 109 aa, synthetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKR

SEQ ID NO: 47 - protein, 125 aa, synthetic construct
EVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSS

SEQ ID NO: 48 - protein, 330 aa, synthetic construct
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 49 - protein, 106 aa, synthetic construct
TVAAPSVFIFPPSDEQLKSGTASVVCLLNNFYPREAKVQWKVDNALQSGNSQESVTEQDSKDSTYSLESTLTLSKADYEKHKVYACEVTHQGLSSPVTKSFNRGEC

SEQ ID NO: 50 - protein, 330 aa, synthetic construct
ASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 51 - protein, 105 aa, synthetic construct
QPKAAPSVTLFPPSSEELQANKATLVCLISDFYPGAVTVAWKADSSPVKAGVETTTPSKQSNNKYAAKSYLSLTPEQWKSHRSYSCQVTHEGSTVEKTVAPTECS

SEQ ID NO: 52 - protein, 327 aa, synthetic construct
ASTKGPSVFPLAPCSRSTSESTAALGCLVKDYFPEPVTVSWNSGALTSGVHTFPAVLQSSGLYSLKSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 327 aa, synthetic construct
ASTKGPSVFPLAPCSRSTSESTAALGCLVKDYFPEPVTVSWNSGALTSGVHTFPAVLQSSGLYSLESVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 54 - protein, 470 aa, synthetic construct
MDMRVPAQLLGLLLLWLRGARCQVQLVESGGGAVQPGRSLRLSCAASGFTFSNYGMHWVRQAPGKGLEWVAVISHHGSSKYYARSVKGRFTISRDNSKNTLYLEMNSLRAEDTAVYYCARDWWELVFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 55 - protein, 241 aa, synthetic construct
MDMRVPAQLLGLLLLWLRGARCDIVMTQTPLSLSVTPGQPASISCKSSQSLLHSDGKTFLYWYLQKPGQPPQLLIYEVSNRFSGVPDRFSGSGSGTDFTLKISRVEAEDVGVYYCLQGIHLPFTFGPGTKVEIKRTVAAPSVFIFPPSDEQLKSGTASVVCLLNNFYPREAKVQWKVDNALQSGNSQESVTEQDSKDSTYSLESTLTLSKADYEKHKVYACEVTHQGLSSPVTKSFNRGEC

SEQ ID NO: 56 - protein, 477 aa, synthetic construct
MDMRVPAQLLGLLLLWLRGARCEVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAENIGTSYISYWAYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 57 - protein, 234 aa, synthetic construct
MAWALLLLTLLTQGTGSWAQTVVTQEPSLTVSPGGTVTITCGSSTGAVTSGNYPNWVQKKPGQAPRGLIGGTKFLAPGTPARFSGSLSGGKAALTLSGVQPEDEAEYYCVLWYSNRWVFGSGTKLTVLGQPKAAPSVTLFPPSSEELQANKATLVCLISDFYPGAVTVAWKADSSPVKAGVETTTPSKQSNNKYAAKSYLSLTPEQWKSHRSYSCQVTHEGSTVEKTVAPTECS

SEQ ID NO: 58 - protein, 470 aa, synthetic construct
MDMRVPAQLLGLLLLWLRGARCQVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQDSLSLSPGK

SEQ ID NO: 59 - protein, 237 aa, synthetic construct
MDMRVPAQLLGLLLLWLRGARCEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKRTVAAPSVFIFPPSDEQLKSGTASVVCLLNNFYPREAKVQWKVDNALQSGNSQESVTEQDSKDSTYSLESTLTLSKADYEKHKVYACEVTHQGLSSPVTKSFNRGEC

SEQ ID NO: 60 - protein, 467 aa, synthetic construct
MDMRVPAQLLGLLLLWLRGARCQVQLVESGGGAVQPGRSLRLSCAASGFTFSNYGMHWVRQAPGKGLEWVAVISHHGSSKYYARSVKGRFTISRDNSKNTLYLEMNSLRAEDTAVYYCARDWWELVFDYWGQGTLVTVSSASTKGPSVFPLAPCSRSTSESTAALGCLVKDYFPEPVTVSWNSGALTSGVHTFPAVLQSSGLYSLKSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - skipped

SEQ ID NO: 62 - DNA, 1413 bp, synthetic construct
  source 1..1413: mol_type = other DNA
atggacatgagggtgcccgctcagctcctggggctcctgctgctgtggctgagaggtgcgcgctgtcaagttcaacttgtcgaatcaggtggtggggcagttcaaccagggcgctcattgcggttgtcatgtgcagcatcaggttttacttttagtaattatgggatgcattgggttcgccaagcacctggaaaaggcctcgaatgggtcgcagtaattagtcatcatggaagttccaaatattatgcaagaagcgtcaaaggtcgctttactatttcacgcgataatagtaagaacacactgtatcttgagatgaattcactcagagcagaagatactgctgtctattattgtgctagagattggtgggaacttgtatttgattattggggacaaggaacgctcgtcacagtatcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcaagagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcaggacagcctctccctgtctccgggcaaatag

SEQ ID NO: 63 - DNA, 726 bp, synthetic construct
  source 1..726: mol_type = other DNA
atggacatgagggtgcccgctcagctcctggggctcctgctgctgtggctgagaggtgcgcgctgtgatattgttatgacacaaacacctctttcactttctgtaacaccgggacaacctgcttccatttcttgtaaatccagtcaaagtctcttgcatagtgatgggaaaacgtttctctattggtatttgcaaaagcctggacaaccaccgcaacttttgatttatgaagtcagtaatcgctttagtggagttccagatcgcttttcaggttctgggtcaggaacagattttacgctcaaaatctcccgggttgaagctgaagatgttggggtctattattgtcttcaagggattcatcttccgtttacttttgggccagggactaaagtcgaaattaaacggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcgaaagcaccctgacgctgagcaaagcagactacgagaaacacaaagtctacgcctgcgaagtcacccatcagggcctgagctcgcccgtcacaaagagcttcaacaggggagagtgttag

SEQ ID NO: 64 - DNA, 1434 bp, synthetic construct
  source 1..1434: mol_type = other DNA
atggacatgagggtgcccgctcagctcctggggctcctgctgctgtggctgagaggtgcgcgctgtgaagtgcagctggttgaatctggcggcggattggttcagcctggcggatctctgaagctgtcttgtgccgcctctggcttcaccttcaacaaatacgccatcaactgggtccgacaggcccctggcaaaggactggaatgggtcgcccggatcagatccaagtacaacaactacgctacctactacgccgacgccgtgaaggaccggttcaccatctccagagatgactccaagaacaccgtctacctgcagatgaacaacctgaaaaccgaggacaccgccgtgtactactgtgcgcgggccgagaacatcggaacctcctacatctcctactgggcctactggggccagggcacactggtcacagttagttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcgagagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 65 - DNA, 705 bp, synthetic construct
  source 1..705: mol_type = other DNA
atggcctgggctctgctgctcctcaccctcctcactcagggcacagggtcctgggcccaaacagtggtcacccaagagcctagcctgaccgtttctcctggcggcacagtgaccatcacctgtggatcttctaccggcgctgtgacctccggcaactaccctaattgggtgcagaagaagcccggccaggctcctagaggactgatcggaggcaccaagtttctggctcccggcactcctgccagattctctggatctctgtccggcggaaaggccgctctgacattgtctggtgtccagcctgaggacgaggctgagtactattgcgtgctgtggtactccaacagatgggtgttcggctccggcaccaagctgacagttctcggacagcccaaggctgcaccctcggtcactctgttcccgccctcctctgaggagcttcaagccaacaaggccacactggtgtgtctcatcagtgacttctacccgggagccgtgacagtggcctggaaggcagatagcagccccgtcaaggcgggagtggaaaccaccacaccctccaaacaaagcaacaacaagtacgcggccaagagctatctgagcctgacgcctgagcagtggaagtcccacagaagctacagctgccaggtcacgcatgaagggagcaccgtggagaagacagtggcccctacagaatgttcatag

SEQ ID NO: 66 - DNA, 1413 bp, synthetic construct
  source 1..1413: mol_type = other DNA
atggacatgagggtgcccgctcagctcctggggctcctgctgctgtggctgagaggtgcgcgctgtcaagtccaacttcaagaatcaggccccgggcttgtcaaaccatctgaaacgctttcactcacttgtacggtcagtggtgggtccatttcttcatattattggagttggattcggcaaccaccaggaaaaggtttggaatggattggttatgtctattattctggcactacgaattataatccgtcacttaaatcccgcgtcactatttccgtcgatacttcaaagaatcaatttagtctcaaactttcctcagtcacggctgctgatactgctgtttattattgtgctagtattgcagtcactgggttttattttgattattggggacaaggtactctcgtcacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcaagagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcaggacagcctctccctgtctccgggcaaatag

SEQ ID NO: 67 - DNA, 714 bp, synthetic construct
  source 1..714: mol_type = other DNA
atggacatgagggtgcccgctcagctcctggggctcctgctgctgtggctgagaggtgcgcgctgtgaaattgtactcacgcaatcacctggtacgctttcactctcccctggtgaacgagtcactcttagttgtcgggcttcacaacgcgtcaataataattatcttgcttggtatcaacaacgaccaggacaagctccacgcttgctcatttatggtgcaagtagtagagcaactggaattcctgatcggttttccgggtctgggagtggaactgattttacattgacgatttctcgcctcgaaccagaagattttgcagtttattattgtcaacaatatgatagatcccctctcacttttggcggtggaactaaacttgaaatcaaacgcacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcgaaagcaccctgacgctgagcaaagcagactacgagaaacacaaagtctacgcctgcgaagtcacccatcagggcctgagctcgcccgtcacaaagagcttcaacaggggagagtgttag

SEQ ID NO: 68 - DNA, 1404 bp, synthetic construct
  source 1..1404: mol_type = other DNA
atggacatgagggtgcccgctcagctcctggggctcctgctgctgtggctgagaggtgcgcgctgtcaagttcaacttgtcgaatcaggtggtggggcagttcaaccagggcgctcattgcggttgtcatgtgcagcatcaggttttacttttagtaattatgggatgcattgggttcgccaagcacctggaaaaggcctcgaatgggtcgcagtaattagtcatcatggaagttccaaatattatgcaagaagcgtcaaaggtcgctttactatttcacgcgataatagtaagaacacactgtatcttgagatgaattcactcagagcagaagatactgctgtctattattgtgctagagattggtgggaacttgtatttgattattggggacaaggaacgctcgtcacagtatcttctgcctccaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcaagagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggcaaatag

SEQ ID NO: 69 - skipped

SEQ ID NO: 70 - DNA, 705 bp, synthetic construct
  source 1..705: mol_type = other DNA
atggcctgggctctgctgctcctcaccctcctcactcagggcacagggtcctgggcccaaacagtggtcacccaagagcctagcctgaccgtttctcctggcggcaccgtgaccatcacctgtggatcttctaccggcgctgtgacctccggcaactaccctaattgggtgcagaagaagcccggccaggctcctagaggactgatcggaggcaccaagtttctggctcccggcactcctgccagattctccggttctctgtctggcggaaaggccgctctgacattgtctggcgtgcagcctgaggatgaggctgagtactattgcgtgctgtggtactccaacagatgggtgttcggctccggcaccaagctgacagttctcgggcagcccaaggctgcaccctcggtcactctgttcccgccctcctctgaggagcttcaagccaacaaggccacactggtgtgtctcatcagtgacttctacccgggagccgtgacagtggcctggaaggcagatagcagccccgtcaaggcgggagtggaaaccaccacaccctccaaacaaagcaacaacaagtacgcggccaagagctatctgagcctgacgcctgagcagtggaagtcccacagaagctacagctgccaggtcacgcatgaagggagcaccgtggagaagacagtggcccctacagaatgttcatag

SEQ ID NO: 71-131 - skipped (61 entries)

SEQ ID NO: 132 - protein, 477 aa, synthetic construct
MDMRVPAQLLGLLLLWLRGARCEVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAGNFGSSYISYWAYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 133 - protein, 474 aa, synthetic construct
MDMRVPAQLLGLLLLWLRGARCEVQLVESGGGLVQPGGSLKLSCAASGFTFNKYAINWVRQAPGKGLEWVARIRSKYNNYATYYADAVKDRFTISRDDSKNTVYLQMNNLKTEDTAVYYCARAENIGTSYISYWAYWGQGTLVTVSSASTKGPSVFPLAPCSRSTSESTAALGCLVKDYFPEPVTVSWNSGALTSGVHTFPAVLQSSGLYSLESVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 134 - DNA, 1434 bp, synthetic construct
  source 1..1434: mol_type = other DNA
atggacatgagggtgcccgctcagctcctggggctcctgctgctgtggctgagaggtgcgcgctgtgaagtgcagctggttgaatctggcggcggattggttcagcctggcggatctctgaagctgtcttgtgccgcctctggcttcaccttcaacaaatacgccatcaactgggtccgacaggcccctggcaaaggactggaatgggtcgcccggatcagatccaagtacaacaactacgctacctactacgccgacgccgtgaaggaccggttcaccatctccagagatgactccaagaacaccgtgtacctgcagatgaacaacctcaagaccgaggacaccgccgtgtactactgtgccagagccggcaacttcggctcctcctacatcagctactgggcctattggggccagggcacactggtcacagttagttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcgagagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggcaaatag

SEQ ID NO: 135 - DNA, 1425 bp, synthetic construct
  source 1..1425: mol_type = other DNA
atggacatgagggtgcccgctcagctcctggggctcctgctgctgtggctgagaggtgcgcgctgtgaagtgcagctggttgaatctggcggcggattggttcagcctggcggatctctgaagctgtcttgtgccgcctctggcttcaccttcaacaaatacgccatcaactgggtccgacaggcccctggcaaaggactggaatgggtcgcccggatcagatccaagtacaacaactacgctacctactacgccgacgccgtgaaggaccggttcaccatctccagagatgactccaagaacaccgtctacctgcagatgaacaacctgaaaaccgaggacaccgccgtgtactactgtgcgcgggccgagaacatcggaacctcctacatctcctactgggcctactggggccagggcacactggtcacagttagttcagcctccaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcgaaagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgagctgcgccgtcaaaggcttctaccccagcgacatcgccgtggagtgggagagcaatgggcagccggagaacaactacaagaccacgcctcccgtgctggactccgacggctccttcttcctcgtgagcaggctaaccgtggacaagagcaggtggcaggaggggaatgtcttctcatgctccgtgatgcatgaggctctgcacaaccactacacacagaagagcctctccctgtctctgggcaaatag